(19) United States
(12) Patent Application Publication
(10) Pub. No.: US 2025/0223620 A1
(43) Pub. Date: Jul. 10, 2025

(54) NOVEL PROCESSES FOR THE PRODUCTION OF POLYNUCLEOTIDES INCLUDING OLIGONUCLEOTIDES

(71) Applicant: GLAXOSMITHKLINE INTELLECTUAL PROPERTY DEVELOPMENT LIMITED, Stevenage (GB)

(72) Inventors: Andreas CRAMERI, Stevenage (GB); Douglas FUERST, Collegeville, PA (US); Joseph HOSFORD, Stevenage (GB); David TEW, Stevenage (GB)

(73) Assignee: GLAXOSMITHKLINE INTELLECTUAL PROPERTY DEVELOPMENT LIMITED, Stevenage (GB)

(21) Appl. No.: 18/853,613

(22) PCT Filed: Apr. 6, 2023
(86) PCT No.: PCT/EP2023/059142

§ 371 Date: Oct. 2, 2024

Related U.S. Application Data
(60) Provisional application No. 63/432,466, filed on Dec. 14, 2022.
(60) Provisional application No. 63/328,912, filed on Apr. 8, 2022.

(51) Int. Cl. C12P 19/34

(52) U.S. Cl. CPC C12P 19/34

(57) ABSTRACT

The invention relates to novel processes using enzymes for the production of polynucleotides or oligonucleotides, wherein said processes are suitable for use in the production of modified polynucleotides or oligonucleotides, such as those for use in therapy.

### FIELD OF THE INVENTION

The invention relates to novel processes using polymerases and ligases for the production of polynucleotides, including oligonucleotides, wherein said processes are suitable for use in the production of modified polynucleotides, including modified oligonucleotides, such as those for use in therapy.

### BACKGROUND TO THE INVENTION

The chemical synthesis of polynucleotides and modified polynucleotides, in particular oligonucleotides and modified oligonucleotides, via for example phosphoramidite chemistry, is well established and has been the method of choice for synthesizing these defined sequence biopolymers for several decades. The synthetic process is usually run as a solid-supported synthesis (commonly referred to as solid-phase synthesis), whereby single nucleotides are added sequentially with the addition of each nucleotide requiring a cycle of several chemical steps to add and deprotect the growing oligonucleotide (“oligo”) in preparation for the subsequent step. At the end of the sequential addition of nucleotides, the oligo is released from the solid phase support, further deprotection takes place, and then the crude oligonucleotide is further purified by column chromatography.

While this method may be considered routine and can be automated, there are several shortcomings to this methodology, especially if the goal is to prepare oligonucleotides and polynucleotides at large-scale as would be needed for oligonucleotide therapeutics, such as antisense molecules, including gapmers, siRNA, miRNA and aptamers, and polynucleotide therapeutics, such as therapeutic mRNA. These shortcomings include scale-up limitations of solid-supported chemistry limiting batch sizes, and practical limitations in the use of chromatography for purifying large quantities of oligonucleotide. These limitations make scale-up expensive and lengthy, requiring multiple rounds of synthesis. Additionally, errors accumulate with the length of the oligonucleotide or polynucleotide being synthesised, placing a further practical limitation on scale-up of longer oligonucleotide and polynucleotide products.

There is a need, therefore, to both reduce or ideally eliminate both chemical synthesis and solid-supported synthesis, and perform a synthesis which can operate efficiently, cost effectively and at larger scales, in order to produce oligonucleotides and longer polynucleotides, whilst errors in the sequence are minimised.

WO2018/011067 discloses a novel ligation method where a pool of oligonucleotides are ligated together in a directed manner using a complementary template and ligation method. WO2019/121500 discloses novel processes using enzymes, in particular single-stranded ligases and transferases, in the production of modified oligonucleotides, which can be used in the ligation methods.

The process for adding each nucleotide to the chain using single-stranded ligases and transferases is complex and must be performed sequentially with one nucleotide added at a time, with deprotection steps required in each round to facilitate the addition, which is time consuming. The individual oligonucleotides in the pool must then each be ligated to one another in the correct orientation using a template to produce the final oligonucleotide product. The scale-up of this process to larger polynucleotides means it is necessary to create multiple short oligonucleotides by adding one nucleotide at a time, which must then be ligated in the correct orientation to produce the final product. Essentially, every single nucleotide of the final product must be added one at a time in creating the smaller oligonucleotides, which are ligated to create the final product, which reduces efficiency and requires a long production process. The risk of error introduction thus becomes higher. There is a need, therefore, to provide an alternative and/or improved method that reduces the complexity in order to increase the efficiency of production of oligonucleotides and larger polynucleotides, whilst errors in the product sequence are minimised.

### SUMMARY OF THE INVENTION

In a first aspect on the invention, a process for producing a single-stranded polynucleotide product having at least one modified nucleotide residue is provided, wherein the process comprises:
a) contacting a template polynucleotide, which comprises a sequence complementary to the single-stranded polynucleotide product, with a pool of at least two segment polynucleotides under conditions to allow annealing of the at least two segment polynucleotides to the template polynucleotide to generate a template polynucleotide with the at least two segment polynucleotides annealed thereto, wherein at least one sequence gap is formed between the at least two annealed segment polynucleotides;
b) extending at least one of the annealed segment polynucleotides using a pool of nucleoside triphosphates and a polymerase, to fill in the at least one sequence gap to generate at least one extended segment polynucleotide;
c) ligating segment polynucleotide(s) and/or extended segment polynucleotide(s) using a ligase to form the single-stranded polynucleotide product bound to the template polynucleotide in a duplex; and
d) changing the conditions to denature the duplex comprising the single-stranded polynucleotide product and the template polynucleotide, whereby the single-stranded polynucleotide product is produced.

In a second aspect of the invention, a process for producing a double-stranded polynucleotide product is provided, wherein the process comprises annealing two complementary single-stranded polynucleotide products, at least one of which has been produced by the process for producing a single-stranded polynucleotide as disclosed herein, optionally wherein both of which have been produced by the process for producing a single-stranded polynucleotide as disclosed herein.

In a third aspect of the invention, a process for producing a double-stranded polynucleotide product is provided, wherein the process comprises:
a) contacting a template polynucleotide, which comprises a sequence complementary to a single-stranded polynucleotide product, with a pool of at least two segment polynucleotides under conditions to allow annealing of the at least two segment polynucleotides to the template polynucleotide to generate a template polynucleotide with the at least two segment polynucleotides annealed thereto, wherein at least one sequence gap is formed between the at least two annealed segment polynucleotides;
b) extending at least one of the annealed segment polynucleotides using a pool of nucleoside triphosphates and a polymerase, to fill in the at least one sequence gap to generate at least one extended segment polynucleotide;
c) ligating segment polynucleotide(s) and/or extended segment polynucleotide(s) using a ligase to form the single-stranded polynucleotide product bound to the template polynucleotide in a duplex;
d) changing the conditions to denature the duplex comprising the single-stranded polynucleotide product and the template polynucleotide, whereby the single-stranded polynucleotide product is produced; and
e) using the single-stranded polynucleotide product as the template polynucleotide in step a) and repeating steps a) to c) to produce the double-stranded polynucleotide product.

### BRIEF DESCRIPTION OF THE DRAWINGS

FIG. 1 is a schematic example showing production of an oligonucleotide or polynucleotide product using a polymerase, including the steps of ligating the segment oligonucleotides or polynucleotides to form the product and changing the conditions to remove impurities.

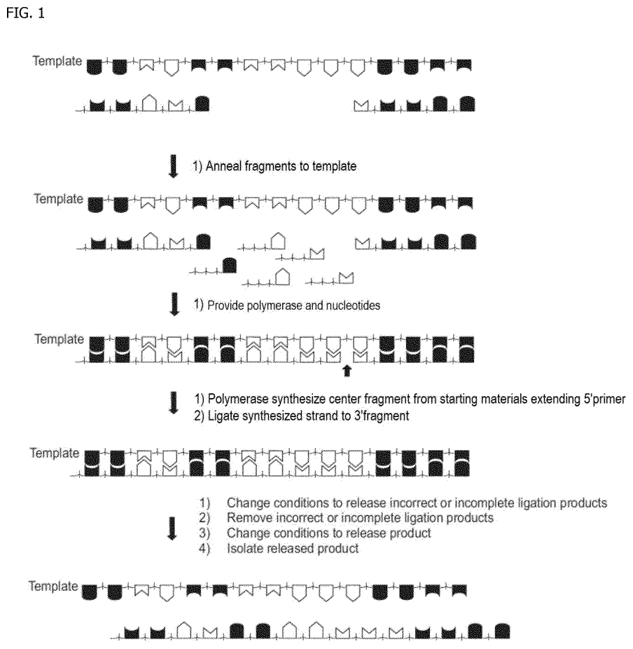

FIG. 2 is a schematic example of multiple template configurations.

FIG. 3a illustrates HPLC trace showing gap filling reaction starting materials, namely a template and two segment oligonucleotides (Primer N which acts as a primer for a polymerase and 3′block 1 which acts as a stopper).

FIG. 3b illustrates HPLC trace showing product formation following gap filling reaction. Primer N+5 indicates successful extension of Primer N using a polymerase and nucleoside triphosphates to fill in a 5 bp gap.

FIG. 4 is a schematic example showing a template with an annealed 5′ primer, and separately a template with a hairpin loop that acts as a 5′ primer.

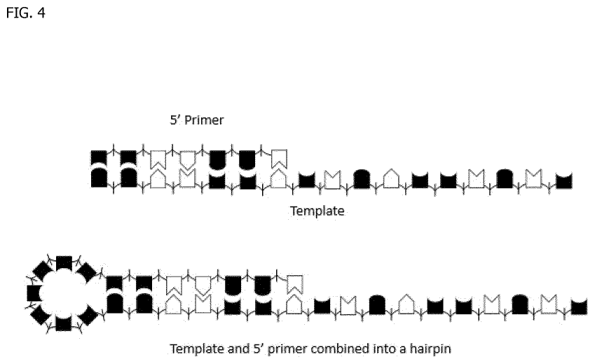

### DETAILED DESCRIPTION OF THE INVENTION

### Definitions

As used herein, “polynucleotide” means two or more nucleotide monomers connected to each other through covalent bonds, i.e. a polymer of nucleotide residues. A single polynucleotide molecule can, for example, comprise 14 or more monomers of nucleotides in a chain structure. DNA and RNA are examples of polynucleotides. Polynucleotides include oligonucleotides. Polynucleotides can contain an infinite number of nucleotides. Polynucleotides are useful therapeutically, for example, in the production of therapeutic mRNA (which can be used as mRNA vaccines), antisense oligonucleotides, siRNAs, miRNAs, aptamers, CRISPR guide RNAs, and oligonucleotides to recruit and guide DNA and RNA editing enzymes, such as A to I RNA base-editing oligonucleotides (AIMers).

As used herein, the term “therapeutic polynucleotide” means a polynucleotide that has a therapeutic application, e.g. in the prevention or treatment of a condition or disease in a human or animal. Such a polynucleotide typically contains one or more modified nucleotide residues or linkages. Therapeutic polynucleotides act via one of several different mechanisms, including, but not limited to, antisense, splice-switching or exon-skipping, immunostimulation, RNA interference (RNAi), e.g. via microRNA (miRNA) and small interfering RNA (siRNA), and recruitment and guiding of DNA and RNA editing enzymes. A therapeutic polynucleotide may be an aptamer. Therapeutic polynucleotides will usually, but not always, have a defined sequence. Therapeutic polynucleotides include therapeutic oligonucleotides.

The terms “polynucleotide” and “therapeutic polynucleotide” encompass “oligonucleotides” and “therapeutic oligonucleotides”, respectively.

As used herein, the term “oligonucleotide”, or “oligo” for short, means a polymer of nucleotide residues. The term “oligonucleotide” is usually used for shorter polynucleotide sequences than the term “polynucleotide”, generally in the range of 3 to 30 nucleotides. These may be deoxyribonucleotides (wherein the resulting oligonucleotide is DNA), ribonucleotides (wherein the resulting oligonucleotide is RNA), modified nucleotides, or a mixture thereof.

A polynucleotide or an oligonucleotide may be entirely composed of nucleotide residues as found in nature (i.e. “natural nucleotides” or “naturally occurring nucleotides”) or may contain at least one modified nucleotide, or at least one linkage between nucleotides that has been modified. Examples of naturally occurring nucleotides include deoxyadenosine monophosphate, deoxycytidine monophosphate, deoxyguanosine monophosphate, deoxythymidine monophosphate, deoxyuridine monophosphate, adenosine monophosphate, cytidine monophosphate, guanosine monophosphate, thymidine monophosphate and uridine monophosphate. A modified nucleotide is not a naturally occurring nucleotide (i.e. it is a non-natural nucleotide). A modified nucleotide may be a naturally occurring nucleotide that has been modified, for example chemically modified. Modified nucleotides may comprise modified backbones, sugars, and/or nucleobases. It is acknowledged that certain modifications occur sporadically in nature, i.e. in naturally occurring nucleotides, such as 2′OMe or C5 pyrimidine modifications, however, in the present disclosure these are considered modified nucleotides. Polynucleotides, including oligonucleotides, can be single-stranded or double-stranded. A polynucleotide or an oligonucleotide of the disclosure may be conjugated to another molecule, e.g. N-Acetylgalactosamine (GalNAc) or multiples thereof (GalNAc clusters).

As used herein, the term “therapeutic oligonucleotide” means an oligonucleotide that has a therapeutic application, e.g. in the prevention or treatment of a condition or disease in a human or animal. Such an oligonucleotide typically contains one or more modified nucleotide residues or linkages. Therapeutic oligonucleotides act via one of several different mechanisms, including, but not limited to, antisense, splice-switching or exon-skipping, immunostimulation and RNA interference (RNAi), e.g. via microRNA (miRNA) and small interfering RNA (siRNA). A therapeutic oligonucleotide may be an aptamer. Therapeutic oligonucleotides will usually, but not always, have a defined sequence. A therapeutic oligonucleotide is an example of a therapeutic polynucleotide.

As used herein, the term “template” means a polynucleotide or oligonucleotide that comprises a sequence complementary to a single-stranded polynucleotide or oligonucleotide product. The template can comprise a sequence which is 100% complementary to the sequence of the target (or product) polynucleotide or oligonucleotide. The template can consist of a sequence that is 100% complementary to the sequence of the target (or product) polynucleotide or oligonucleotide. The template can be a longer sequence compared to the product sequence. The template can comprise sequences which are not used to produce the polynucleotide or oligonucleotide product. When the template is longer in sequence compared to the product sequence a stopper can be used to control production and/or length of the polynucleotide or oligonucleotide product. When the template is longer in sequence compared to the product sequence a primer can be used to control production and/or length of the polynucleotide or oligonucleotide product. When the template is longer in sequence compared to the product sequence it may comprise a hairpin loop. The hairpin loop may act as a primer. The template may comprise one of the segments in a hairpin loop. In such instances, the product may be released from the template via cleavage with a nuclease, a nickase, a DNAzyme, or chemical methods. The template can be a shorter sequence compared to the product sequence. When the template is a shorter sequence compared to the product sequence, at least one segment will overhang the template when the segment is annealed to the template. The template can comprise or consist of a sequence that is less than 100% complementary. The template sequence can be such that the respective complementary nucleotides are complementary to the unmodified form of modified nucleotides in the target sequence. Unless otherwise specified, as used herein, the term “complementary” means 100% complementary.

As used herein, the term “product” means the desired polynucleotide or oligonucleotide, having a specific sequence and set of modifications, also referred to herein as a “target polynucleotide” or “target oligonucleotide”. “Product sequence” is used interchangeably with “target polynucleotide sequence” and “target oligonucleotide sequence” and refers to the base sequence of the product.

As used herein, the term “pool of polynucleotides or oligonucleotides” refers to a group of polynucleotides or oligonucleotides, respectively, that may vary in sequence, may be shorter than the target sequence, and may not have the same sequence as the target sequence. The pool of polynucleotides or oligonucleotides may be the product of polynucleotide or oligonucleotide synthesis. The pool of polynucleotides or oligonucleotides may comprise at least two segment polynucleotides or oligonucleotides. The at least two segment polynucleotides or oligonucleotides may be non-random (i.e. not a group of randomly selected segment polynucleotides or oligonucleotides, but specifically designed and selected for the purpose of forming the final polynucleotide product in accordance with the process of the present invention). The at least two segment polynucleotides or oligonucleotides may be segments of the polynucleotide or oligonucleotide product. The at least two segment polynucleotides or oligonucleotides may be different in sequence. The pool of polynucleotides or oligonucleotides may comprise segments of the product sequence. The pool of polynucleotides or oligonucleotides may consist of segments of the product sequence. The pool of polynucleotides or oligonucleotides may be engineered to specifically comprise segments of the polynucleotide or oligonucleotide product. At least one segment of the product sequence can contain at least one modified nucleotide residue. The pool of polynucleotides or oligonucleotides can be the product of polynucleotide or oligonucleotide synthesis using a polymerase, such as a DNA polymerase or an RNA polymerase. One or more, or all segment polynucleotides or oligonucleotides can be made using, e.g. chemical synthesis e.g. solid-supported or solution phase synthesis, such as via phosphoramidite chemistry, or can be made using enzymatic synthesis, or combinations thereof. Enzymatic synthesis can involve the use of a polymerase, a single-stranded ligase and/or a transferase or combinations thereof. The pool of polynucleotides or oligonucleotides can be the product of polynucleotide or oligonucleotide synthesis using chemical synthesis e.g. solid supported or solution phase synthesis, such as via phosphoramidite chemistry, or using enzymatic synthesis, or combinations thereof. Enzymatic synthesis can involve the use of a polymerase, a single-stranded ligase and/or a transferase or combinations thereof.

As used herein, the term “annealing” means the hybridisation of complementary polynucleotides or oligonucleotides in a sequence specific manner, e.g. the pairing of two single-stranded polynucleotides or oligonucleotides, via the hydrogen bonds of Watson and Crick base-pairing, to form a double-stranded polynucleotide or oligonucleotide (a “duplex”). “Conditions to allow for annealing” will depend on the Tₘ of the hybridised complementary polynucleotides or oligonucleotides and will be readily apparent to a person skilled in the art. For example, the temperature for annealing may be below the Tₘ of the hybridised polynucleotides or oligonucleotides. Alternatively, the temperature for annealing may be close to the Tₘ of the hybridised polynucleotides or oligonucleotides, e.g. +/−1, 2 or 3° C. The temperature for annealing is, in general, not higher than 10° C. above the Tₘ of the hybridised polynucleotides or oligonucleotides.

As used herein, the term “denaturing” in relation to a double-stranded polynucleotide or oligonucleotide is used to mean that the complementary strands are no longer annealed, i.e. the Watson and Crick base-pairing has been disrupted and the strands have dissociated. Denaturing occurs as a result of changing the conditions, for example, by raising the temperature, changing the pH, or changing the salt concentration of the buffering solution. Conditions for denaturing are well known to those skilled in the art. Denaturing a double-stranded polynucleotide or oligonucleotide (i.e. denaturing a duplex) as described herein results in a single-stranded product, or impurity polynucleotide or oligonucleotide, and a single-stranded template polynucleotide or oligonucleotide.

As used herein, the term “impurity” or “impurities” means polynucleotides or oligonucleotides that do not have the desired product sequence. These polynucleotides or oligonucleotides may include polynucleotides or oligonucleotides that are shorter than the product (for example 1, 2, 3, 4, 5 or more nucleotide residues shorter), or that are longer than the product (for example 1, 2, 3, 4, 5 or more nucleotide residues longer). Where the production process includes a step whereby linkages are formed between segments, impurities include polynucleotides or oligonucleotides that are remaining if one or more of the linkages fail to form. Impurities also include polynucleotides or oligonucleotides where incorrect nucleotides have been incorporated, resulting in a mismatch when compared to the template. An impurity may have one or more of the characteristics described above. The terms “impurity” and “impurity polynucleotide” are used interchangeably herein.

As used herein, the term “segment” is a smaller portion of a longer polynucleotide or oligonucleotide, in particular a smaller portion of a product or target polynucleotide or oligonucleotide. For a given product, when all of its segments are annealed to its template, gaps are filled by polymerase extension and ligated together, the product is formed. A segment may act as a primer for the polymerase. A segment may act as a stopper for the polymerase. A segment may be part of a hairpin loop in the template that is then cleaved from the template, following polymerase extension, and is part of the product.

As used herein, the term “enzymatic ligation” means that the link between two adjacent nucleotides is formed enzymatically, i.e. by an enzyme. This linkage may be a naturally occurring phosphodiester bond (PO), or a modified linkage including, but not limited to, phosphorothioate (PS), phosphoramidate (PA) or phosphorodithioate (PS2).

As used herein, the term “enzymatic synthesis” means the production of polynucleotides and oligonucleotides, including segments and final product, using enzymes, e.g. polymerases, ligases, transferases, phosphatases, and nucleases e.g. endonucleases. These enzymes may be wild-type enzymes or mutant enzymes or engineered enzymes. Within the scope of the present disclosure are mutant enzymes or engineered enzymes capable of acting on modified nucleotide or oligonucleotide substrates.

As used herein, the term “polymerase” means an enzyme that catalyses the joining, i.e. covalent joining, of a nucleotide to the 3′-OH of another nucleotide or oligonucleotide or polynucleotide e.g. by formation of a phosphodiester bond between the 3′ end of one nucleotide or oligonucleotide or polynucleotide and the 5′ end of another nucleotide. Accordingly, polymerase activity is 5′ to 3′. The polymerase can include DNA and/or RNA polymerases. The polymerase may be a wild-type enzyme, mutant enzyme or engineered enzyme.

The skilled person understands that the “pool of nucleotides” as used in the present disclosure are substrates for the polymerase. Therefore, the “pool of nucleotides” in this context means a pool of nucleoside triphosphates (NTPs) or analogs thereof, which when incorporated into the polynucleotide or oligonucleotide product are nucleotides. Accordingly, “pool of nucleotides” and “pool of nucleoside triphosphates” are used interchangeably herein. Nucleoside triphosphates may be regarded as the molecular precursors of both DNA and RNA. The pool of nucleoside triphosphates may comprise one or more of: deoxyadenosine triphosphate, deoxycytidine triphosphate, deoxyguanosine triphosphate, deoxythymidine triphosphate, deoxyuridine triphosphate, adenosine triphosphate, cytidine triphosphate, guanosine triphosphate, thymidine triphosphate and uridine triphosphate. The pool of nucleoside triphosphates may comprise one or more of: modified deoxyadenosine triphosphate, modified deoxycytidine triphosphate, modified deoxyguanosine triphosphate, modified deoxythymidine triphosphate, modified deoxyuridine triphosphate, modified adenosine triphosphate, modified cytidine triphosphate, modified guanosine triphosphate, modified thymidine triphosphate and modified uridine triphosphate. The pool of nucleoside triphosphates may comprise a nucleoside triphosphate analog. Examples of nucleoside alpha thiotriphosphates include 2′-deoxyadenosine-5′-(α-thio)-triphosphate, 2′-deoxycytidine-5′-(α-thio)-triphosphate, 2′-deoxyguanosine-5′-(α-thio)-triphosphate, 2′-deoxythymidine-(α-thio)-triphosphate, 2′-deoxyuridine-(α-thio)-triphosphate, 2′-adenosine-5′-(α-thio)-triphosphate, 2′-cytidine-5′-(α-thio)-triphosphate, 2′-guanosine-5′-(α-thio)-triphosphate, 2′-thymidine-(α-thio)-triphosphate, 2′-uridine-(α-thio)-triphosphate and modified base variants thereof. The pool of nucleoside triphosphates may comprise one or more of: deoxyadenosine triphosphate, deoxycytidine triphosphate, deoxyguanosine triphosphate, deoxythymidine triphosphate, deoxyuridine triphosphate, adenosine triphosphate, cytidine triphosphate, guanosine triphosphate, thymidine triphosphate, uridine triphosphate, modified deoxyadenosine triphosphate, modified deoxycytidine triphosphate, modified deoxyguanosine triphosphate, modified deoxythymidine triphosphate, modified deoxyuridine triphosphate, modified adenosine triphosphate, modified cytidine triphosphate, modified guanosine triphosphate, modified thymidine triphosphate, modified uridine triphosphate, 2′-deoxyadenosine-5′-(α-thio)-triphosphate, 2′-deoxycytidine-5′-(α-thio)-triphosphate, 2′-deoxyguanosine-5′-(α-thio)-triphosphate, 2′-deoxythymidine-(α-thio)-triphosphate, 2′-deoxyuridine-(α-thio)-triphosphate, 2′-adenosine-5′-(α-thio)-triphosphate, 2′-cytidine-5′-(α-thio)-triphosphate, 2′-guanosine-5′-(α-thio)-triphosphate, 2′-thymidine-(α-thio)-triphosphate and 2′-uridine-(α-thio)-triphosphate. The pool of nucleoside triphosphates may comprise one or more of: adenosine triphosphate, cytidine triphosphate, guanosine triphosphate, uridine triphosphate, modified adenosine triphosphate, modified cytidine triphosphate, modified guanosine triphosphate, modified uridine triphosphate, 2′-adenosine-5′-(α-thio)-triphosphate, 2′-cytidine-5′-(α-thio)-triphosphate, 2′-guanosine-5′-(α-thio)-triphosphate, and 2′-uridine-(α-thio)-triphosphate. The pool of nucleoside triphosphates may comprise one or more of: deoxyadenosine triphosphate, deoxycytidine triphosphate, deoxyguanosine triphosphate, deoxythymidine triphosphate, modified deoxycytidine triphosphate, modified deoxyguanosine triphosphate, modified deoxythymidine triphosphate, 2′-deoxyadenosine-5′-(α-thio)-triphosphate, 2′-deoxycytidine-5′-(α-thio)-triphosphate, 2′-deoxyguanosine-5′-(α-thio)-triphosphate, and 2′-deoxythymidine-(α-thio)-triphosphate. The pool of nucleoside triphosphates may comprise: adenosine triphosphate, cytidine triphosphate, guanosine triphosphate, and modified uridine triphosphate. The pool of nucleoside triphosphates may comprise: adenosine triphosphate, cytidine triphosphate, guanosine triphosphate, and N1-methyl-pseudouridine triphosphate. The pool of nucleoside triphosphates may comprise: 2′-adenosine-5′-(α-thio)-triphosphate, 2′-cytidine-5′-(α-thio)-triphosphate, 2′-guanosine-5′-(α-thio)-triphosphate, and 2′-modified uridine-(α-thio)-triphosphate.

Within the scope of the disclosure is a polymerase capable of joining an unmodified nucleotide to another unmodified nucleotide, a polymerase capable of joining an unmodified nucleotide to a modified nucleotide (i.e. a modified 5′ nucleotide to an unmodified 3′ nucleotide, and/or an unmodified 5′ nucleotide to a modified 3′ nucleotide), as well as a polymerase capable of joining a modified nucleotide to another modified nucleotide. Optionally, the polymerase is capable of joining an unmodified nucleotide to another unmodified nucleotide. The unmodified nucleotide can subsequently be modified. Examples of modifications of nucleotides are disclosed herein, and include modifications selected from the group comprising a modification of the sugar moiety, modification of the nucleobase, and modification of the backbone.

The modification can be at the 2′ position of the sugar moiety, optionally selected from the group consisting of 2′-F, 2′-OMe, 2′-MOE, and 2′-amino. The oligonucleotide can comprise a PMO, a LNA, a c-Et, a PNA, a BNA, or a L-Ribonucleic acid. The modification can be in the nucleobase, and optionally can be selected from the group consisting of a 5-methyl pyrimidine, a 7-deazaguanosine and an abasic nucleotide. The modification can be in the backbone, and optionally can be selected from the group consisting of phosphorothioate, phosphorodithioate phosphoramidate and phosphorodiamidate.

Additionally, exemplary engineered DNA and RNA polymerases that can incorporate modified nucleotides include those disclosed in “Engineering and application of polymerases for synthetic genetics”, Houlihan et al., Current Opinion in Biotechnology 2017, 48; 168-179. For example, DNA and RNA polymerases can be engineered to accept 2′ sugar modifications, including polymerases with mutations in the polymerase thumb subdomain of Thermococcus gogonarius (Tgo) replicative DNA polymerase, optionally comprising mutations at E664K and Y409G. Such polymerases provide for the inclusion of, for example, pseudouridine, 5-methyl-C, 2′-fluoro, or 2-azdio-modified NTPs primed from DNA, RNA, Locked nucleic acid, or 2′-OMe RNA modified nucleotides, or combinations thereof.

Further exemplary RNA polymerases engineered to accept 2′ sugar modifications include T7 RNA polymerases. T7 RNA polymerases comprising a mutation at Y639F can, for example, provide for the inclusion of, for example, 2′fluoro pyrimidines and 2′ amino pyrimidines.

Variants of the Stoffel fragment of Taq polymerase (SM19) can be engineered to accept 2′ sugar modifications. For example, introduction of a negatively charged amino acid at position 614 and mutation of E615G, provide for the inclusion of 2′ sugar modifications. SM19 can be further evolved to polymerase SFM4-3 and SFM4-9. For example, SFM4-3 can transcribe fully modified 2′OMe 60 nucleotide sequences.

Thermophilic RNA polymerase from the marine cyanophage Syn5 can be engineered to accept 2′ sugar modifications.

Tgo polymerases comprising mutations at Y409G, I521L, F545L, and E664K can synthesise DNA and RNA with regioisomeric 2′-5′ linkages by incorporation of 3′deoxy- or 3′OMe nucleotides.

As used herein, the term “ligase” means an enzyme that catalyses the joining, i.e. covalent joining, of two polynucleotide or oligonucleotide molecules, e.g. by formation of a phosphodiester bond between the 3′ end of one polynucleotide or oligonucleotide (or segment) and the 5′ end of the same or another polynucleotide or oligonucleotide (or segment). These enzymes are often referred to as DNA ligases or RNA ligases and utilise cofactors: ATP (eukaryotic, viral and archaeal DNA ligases) or NAD (prokaryotic DNA ligases). Despite their occurrence in all organisms, DNA ligases show a wide diversity of amino acid sequences, molecular sizes and properties (Nucleic Acids Research, 2000, Vol. 28, No. 21, 4051-4058). They are usually members of the Enzyme Class EC 6.5 as defined by the International Union of Biochemistry and Molecular Biology, i.e. ligases used to form phosphoric ester bonds. Within the scope of the disclosure is a ligase capable of joining an unmodified polynucleotide or oligonucleotide to another unmodified polynucleotide or oligonucleotide, a ligase capable of joining an unmodified polynucleotide or oligonucleotide to a modified polynucleotide or oligonucleotide (i.e. a modified 5′ polynucleotide or oligonucleotide to an unmodified 3′ polynucleotide or oligonucleotide, and/or an unmodified 5′ polynucleotide or oligonucleotide to a modified 3′ polynucleotide or oligonucleotide), as well as a ligase capable of joining a modified polynucleotide or oligonucleotide to another modified polynucleotide or oligonucleotide.

As used herein, the term “single-stranded ligase” or “ssLigase” means an enzyme, e.g. an RNA ligase, that is capable of catalysing the ATP-dependent ligation of (i) 5′-phosphorylated single-stranded RNA to the 3′-OH of a single-stranded acceptor RNA strand and (ii) the ligation of a single residue (including a modified residue), e.g. a nucleotide-3′,5′-bisphosphate, 3′,5′-bisthiophosphate or 3′-phosphate-5′thiophosphate, to the 3′ end of RNA or a modified polynucleotide or oligonucleotide (Modified Oligoribonucleotides: 17 (11), 2077-2081, 1978). An example of a ssLigase is T4 RNA ligase, which has also been shown to work on DNA substrates under certain conditions (Nucleic Acids research 7(2), 453-464, 1979). The natural function of T4 RNA ligase in Escherichia coli infected with T4 bacteriophage is to repair single-strand brakes to bacterial tRNA caused by bacterial defence mechanisms against viral attack. Within the scope of the disclosure is a ssLigase capable of joining an unmodified nucleotide to an unmodified polynucleotide or oligonucleotide, a ssLigase capable of joining an unmodified nucleotide to a modified polynucleotide or oligonucleotide, a ssLigase capable of joining a modified nucleotide to an unmodified polynucleotide or oligonucleotide, as well as a ssLigase capable of joining a modified nucleotide to a modified polynucleotide or oligonucleotide. A ssLigase according to the disclosure is a ligase that does not require a template polynucleotide or oligonucleotide for ligation to occur, i.e. the ligation activity of the ligase is template-independent.

As used herein, a “junction nucleotide” is a nucleotide present at the end of one polynucleotide or oligonucleotide that is to be joined to another polynucleotide or oligonucleotide. For example, where two segments, a 5′-segment and a 3′-segment, are to be ligated together, the two junction nucleotides are 1) the nucleotide at the 3′-end of the 5′-segment and 2) the nucleotide at the 5′-end of the 3′-segment.

As used herein, a “transferase” means an enzyme that catalyses template independent joining of one nucleotide to another nucleotide or oligonucleotide. A transferase as described herein includes a terminal nucleotidyl transferase (TdT), also known as DNA nucleotidylexotransferase (DNTT) or terminal transferase. TdT is a specialised DNA polymerase that is expressed in immature, pre-B, pre-T-lymphoid cells where it enables V-D-J antibody gene junctional diversity. TdT catalyses the addition of nucleotides to the 3′ terminus of a DNA molecule. A transferase as described herein includes a non-naturally occurring or mutant TdT. Within the scope of the disclosure is a transferase capable of joining an unmodified nucleotide to an unmodified oligonucleotide, a transferase capable of joining an unmodified nucleotide to a modified oligonucleotide, a transferase capable of joining a modified nucleotide to an unmodified oligonucleotide, as well as a transferase capable of joining a modified nucleotide to a modified oligonucleotide.

As used herein, a “thermostable ligase”, “thermostable polymerase” or “thermostable transferase” is a ligase, polymerase or transferase, respectively, that is active at elevated temperatures, i.e. above human body temperature, i.e. above 37° C. A thermostable ligase, thermostable polymerase or thermostable transferase may be active at, for example, 40° C. to 65° C.; or 40° C. to 90° C.; and so forth.

As used herein, the term “primer” means a polynucleotide or oligonucleotide sequence that is used as a starting point for synthesising a segment polynucleotide or oligonucleotide of the disclosure. A polymerase may require a primer e.g. DNA polymerase may require a primer. A primer may comprise at least 3 nucleotides. It is within the scope of the disclosure to use a segment polynucleotide or oligonucleotide as the primer. At least one segment polynucleotide or oligonucleotide may act as a primer. The primer can be bound to the template before the polymerase catalyses the joining of nucleotides. The primer may not be removed. The primer can form part of the polynucleotide or oligonucleotide template. For example, the template may comprise a hairpin loop which comprises a primer. The primer can form part of the polynucleotide or oligonucleotide product.

As used herein, the term “stopper” also known as “block” or “3′-flanking oligonucleotide” means a polynucleotide or oligonucleotide sequence that stops or prevents polymerase from joining further nucleotides. A stopper may terminate polymerase extension. The stopper, e.g. one of the at least two segment polynucleotides or oligonucleotides, may comprise a 5′ phosphate, 5′ thiophosphate (which may result in a phosphorothioate bond), 5′ amidophosphate (which may result in a phosphoramidate bond), 5′ diamidophosphate (which may result in a phosphorodiamidate bond), 5′ amidothiophosphate, 5′ amidodithiophosphate, 5′ diamidothiophosphate or 5′ dithiophosphate (which may result in a phosphorodithioate bond). A 5′ phosphate, 5′thiophosphate, 5′ amidophosphate, 5′ diamidophosphate, 5′ dithiophosphate, 5′ amidothiophosphate, 5′ amidodithiophosphate or 5′ diamidothiophosphate may be required for ligation with a ligase. One or more of the at least two segment polynucleotides or oligonucleotides may act as a stopper.

The processes of the disclosure can be used to create RNA and/or DNA (including modifications therein), including non-replicating mRNA and virally derived, self-amplifying RNA. Such RNA has utility, for example, in vaccine manufacture.

The term “RNA” is the usual abbreviation for ribonucleic acid. It is a nucleic acid molecule, i.e. a polymer consisting of nucleotide monomers. These nucleotides are usually adenosine-monophosphate, uridine-monophosphate, guanosine-monophosphate and cytidine-monophosphate monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA-sequence. The term “RNA” generally refers to a molecule or to a molecular species selected from the group consisting of long-chain RNA, coding RNA, non-coding RNA, single-stranded RNA (ssRNA), double-stranded RNA (dsRNA), linear RNA (linRNA), circular RNA (circRNA), messenger RNA (mRNA), RNA oligonucleotides, small interfering RNA (siRNA), small hairpin RNA (shRNA), antisense RNA (asRNA), CRISPR/Cas9 guide RNAs, riboswitches, immunostimulating RNA (isRNA), ribozymes, aptamers, ribosomal RNA (rRNA), transfer RNA (tRNA), viral RNA (vRNA), retroviral RNA or replicon RNA, small nuclear RNA (snRNA), small nucleolar RNA (snoRNA), microRNA (miRNA), and a Piwi-interacting RNA (piRNA). Optionally in the context of the disclosure is any type of therapeutic RNA. “Therapeutic RNA” is to be understood as relating to RNA that is suitable for use in the human or animal body for a medical purpose, e.g. it has a clinical grade, particularly when it comes to parameters such as purity, integrity, as well as concerning the underlying production methods that must comply with current good manufacturing practice (cGMP) conditions. A therapeutic RNA may have therapeutic application e.g. in the prevention or treatment of a condition or disease.

The term “messenger RNA” (mRNA) refers to one type of RNA molecule. In vivo, transcription of DNA usually results in the so-called premature RNA which has to be processed into so-called messenger RNA, usually abbreviated as mRNA. Processing of the premature RNA, e.g. in eukaryotic organisms, comprises a variety of different post-transcriptional modifications such as splicing, 5′-capping, polyadenylation, export from the nucleus or the mitochondria and the like. The sum of these processes is also called maturation of mRNA. The mature mRNA usually provides the nucleotide sequence that may be translated into an amino acid sequence of a particular peptide or protein. Typically, a mature mRNA comprises a 5′ cap, a 5′ untranslated region (5′ UTR), an open reading frame, a 3′ untranslated region (3′ UTR) and a homopolymeric tail e.g. a poly-A or a poly-C sequence. In the context of the present disclosure, the mRNA can be an artificial molecule, i.e. a molecule not occurring in nature. This means that the mRNA in the context of the present disclosure may comprise a combination of a 5′ UTR, open reading frame, 3′ UTR and poly-A sequence, which does not occur in this combination in nature.

Depending on the intended therapeutic use of the proteins encoded by the therapeutic mRNA, the dosage and treatment duration of therapeutic mRNAs may vary by orders of magnitude. For vaccines, the expression of nanogram or microgram ranges of an antigen may be sufficient for eliciting the required immune response. However, for growth factors, hormones or antibodies, the therapeutic dose could range from micrograms to milligrams, or potentially up to gram quantities of protein. mRNA dose-dependent toxicity is likely to be a limiting factor to scale-up in order to achieve such large protein quantities, so modifications that lead to increased mRNA stability, without modification-specific toxicity, are of value (Aditham et al., ACS Chem. Biol., December 2021, https://doi.org/10.1021/acschembio.1c00569).

For any application of mRNA in a therapeutic setting, it is desired to use mRNA with a defined sequence and structure that can be reproduced in a reliable manner. For example, the 5′ UTR (e.g. containing a cap structure) and the 3′ UTR (e.g. containing a homopolymeric tail, such as a poly-A tail) of an mRNA are known to be involved in the regulation of mRNA stability and translation efficiency. Accordingly, the 5′ cap structure and the 3′ tail are important features for efficient translation of mRNA and protein synthesis in eukaryotic cells. Therefore, the mRNA production method can be controlled for such key functional features. It has been shown that including 3′ terminal PS linkages in the poly A tail increases protein production by factors of 2-4 in human HeLa cell lines primarily by stabilising mRNAs (Aditham et al., ACS Chem. Biol., December 2021).

The mRNA may have a modified cap. The mRNA may have a 7mG(5′)ppp(5′)N,pN2p (cap 0), 7mG(5′)ppp(5′)NImpNp (cap 1), 7mG(5′)-ppp(5′)NImpN2mp (cap 2) or m(7)Gpppm(3)(6,6,2′)Apm(2′)Apm(2′)Cpm(2)(3,2′)Up (cap 4).

In addition, non-capped RNA typically contains a 5′-terminal triphosphate group that is known to stimulate the innate immune system. Therefore, non-capped RNA may generate undesired immune responses in a subject. Thus, a pharmaceutical mRNA product has to be controlled for the presence of non-capped 5′-triphosphate RNA.

Conventional mRNA-based vaccines encode the antigen of interest and contain 5′ and 3′ UTRs, whereas self-amplifying RNAs encode not only the antigen but also the viral replication machinery that enables intracellular RNA amplification and abundant protein expression. Messenger RNA molecules are typically produced by RNA in vitro transcription of a suitable DNA template. The 5′ cap structure and 3′ homopolymeric tails (e.g. poly-A tails) are typically introduced during RNA in vitro transcription, e.g. can be encoded within the DNA template, or via enzymatic methods after RNA in vitro transcription.

The processes of the disclosure can be used to prepare self-replicating RNA by in vitro transcription. For instance, a DNA-dependent RNA polymerase (such as the bacteriophage T7, T3 or SP6 RNA polymerases) can be used to transcribe the self-replicating RNA from a DNA template.

Appropriate capping and poly-A tail addition reactions via enzymatic methods can be used as required after RNA production using the process of the disclosure or can be encoded within the DNA template.

The polymerases can have precise requirements for the nucleotides they incorporate (i.e. their nucleoside triphosphate substrates). Those requirements can be matched with the requirements of the encoded replicase, to ensure that the transcribed RNA functions as a substrate for its self-encoded replicase.

The processes can be used to prepare non-replicating mRNA. For instance, DNA or RNA polymerases can be used to transcribe the non-replicating mRNA from a DNA or RNA template. The polymerases that can be utilised are described herein. Exemplary polymerases include DNA polymerase I or a T7 RNA polymerase, which can be further mutated as described herein.

In the processes, the RNA can be modified and/or stabilised RNA.

“Stabilised RNA” is defined as RNA showing improved resistance to in vivo degradation and/or an RNA showing improved stability in vivo, and/or an RNA showing improved translatability in vivo.

Stabilisation may be achieved, for example, by a modified phosphate backbone of the produced RNA. A backbone modification is a modification in which phosphates of the backbone of the nucleotides contained in the RNA are chemically modified. Nucleotides that may be used in this connection contain e.g. a phosphorothioate modified phosphate backbone, optionally at least one of the phosphate oxygens contained in the phosphate backbone being replaced by a sulphur atom.

Stabilised RNAs may further include, for example, phosphate analogues, such as, for example, alkyl and aryl phosphonates, or alkylphosphotriesters. Such backbone modifications typically include, without implying any limitation, modifications from the group consisting of methylphosphonates, phosphoramidates and phosphorothioates.

“Modified RNA” includes one or more modified nucleotides.

As used herein, the term “modified nucleotide residue” or “modified polynucleotide or oligonucleotide” means a nucleotide residue or polynucleotide or oligonucleotide which contains at least one aspect of its chemistry that differs from a naturally occurring nucleotide residue or polynucleotide or oligonucleotide. Such modifications can occur in any part of the nucleotide residue, such as modification of the sugar moiety, modification of the nucleobase, and/or modification of the backbone. The modified nucleotide residue can form part of a modified polynucleotide or oligonucleotide. The modified polynucleotide or oligonucleotide can be DNA or RNA.

A person skilled in the art will appreciate that there are many synthetic derivatives of nucleotides.

Additionally, any nucleotide different from G, C, U, T, A may be regarded as a “modified nucleotide”. Examples of modifications of nucleotides are disclosed herein.

The polynucleotide or oligonucleotide can comprise a PMO, a LNA, a c-Et, a PNA, a BNA, or a L-Ribonucleic acid.

A backbone modification in connection with the present disclosure is a modification in which phosphates of the backbone of the nucleotides contained in a nucleic acid are chemically modified. A sugar modification in connection with the present disclosure is a chemical modification of the sugar of the nucleotide(s). A base modification in connection with the present disclosure is a chemical modification of the base moiety of the nucleotide(s). In this context, nucleotide modifications are selected from nucleotide analogues which are applicable for transcription and/or translation.

Modified nucleoside triphosphates known in the art comprise 2-amino-6-chloropurineriboside-5′-triphosphate, 2-aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-fluorothymidine-5′-triphosphate, 2′-0-methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate.

Base-modified nucleotides known in the art include 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyluridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-0-(1-thiophosphate)-adenosine, 5′-0-(1-thiophosphate)-cytidine, 5′-0-(1-thiophosphate)-guanosine, 5′-0-(1-thiophosphate)-uridine, 5′-0-(1-thiophosphate)-pseudouridine, 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thiouridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, Pseudo-iso-cytidine, 6-chloro-purine, N6-methyl-adenosine, alpha-thio-adenosine, 8-azido-adenosine, 7-deaza-adenosine, pseudouridine, N1-methylpseudouridine, N1-ethylpseudouridine, 2-thiouridine, 4′-thiouridine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 2′-0-methyl uridine, pseudouridine (y), N1-methylpseudouridine, 5-methylcytosine, and 5-methoxyuridine.

A skilled person will appreciate that in vitro transcribed (IVT) mRNA triggers a strong immune response upon transfection, which suppresses protein production. Accordingly, 100% replacement of uridine with pseudouridine or N1-methyl pseudouridine is widely used in therapeutic mRNA to reduce immune toxicity through blocking Toll-like receptor recognition, which in turn increases translation efficiency.

The polynucleotide or oligonucleotide of the disclosure can comprise at least one coding sequence, wherein the at least one coding sequence is a pseudouridine modified coding sequence, i.e. every uridine is replaced with pseudouridine in the coding sequence. The polynucleotide or oligonucleotide can comprise a nucleic acid sequence wherein at least one or more than one, or all uridines are replaced by pseudouridines. The polynucleotide or oligonucleotide can comprise at least one coding sequence, wherein the at least one coding sequence is an N1-methylpseudouridine-modified coding sequence, i.e. every uridine is replaced with N1-methylpseudouridine in the coding sequence. N1-methylpseudouridine and 1-methylpseudouridine may be used interchangeably. The polynucleotide or oligonucleotide can comprise a nucleic acid sequence wherein at least one or more than one, or all uridines are replaced by N1-methylpseudouridines. The polynucleotide or oligonucleotide can comprise at least one coding sequence, wherein the at least one coding sequence is a codon-modified coding sequence. The at least one coding sequence can be a codon-modified coding sequence, wherein the amino acid sequence encoded by the at least one codon-modified coding sequence is not modified compared to the amino acid sequence encoded by the corresponding wild-type coding sequence, i.e. the amino acid sequences are identical.

The term “codon-modified coding sequence” relates to coding sequences that differ in at least one codon (triplets of nucleotides coding for one amino acid) compared to the corresponding wild-type coding sequence. Suitably, a codon modified coding sequence may show improved resistance to in vivo degradation and/or improved stability in vivo, and/or improved translatability in vivo. Codon modifications can make use of the degeneracy of the genetic code wherein multiple codons may encode the same amino acid and may be used interchangeably to optimise the coding sequence for in vivo applications.

Modifications in the sugar moiety can include a modification at the 2′ position of the sugar moiety, a bicyclic sugar or a 4′-CH(CH₃)—O-2′ group, and combinations thereof. For example, the modification at the 2′ position of the sugar moiety can comprise a 2′-F, 2′-OMe, 2′-MOE, and/or 2′-amino.

Examples of modified nucleobases include a cytosine, such as a 5-methyl cytosine, a 5-methyl pyrimidine, a 7-deazaguanosine and an abasic nucleotide. Further examples of modified nucleobases include, m5C (5-methylcytidine), m5U (5-methyluridine), m6A (N6-methyladenosine), s2U (2-thiouridine), Um (2′-0-methyluridine), mIA (1-methyladenosine); m2A (2-methyladenosine); Am (2′-0-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6.-hydroxynorvalylcarbamoyl adenosine); ms2hn6A (2-methylthio-N6-hydroxynorvalyl carbamoyladenosine); Ar(p) (2′-0-ribosyladenosine (phosphate)); I (inosine); mi I (1-methylinosine); m′im (1,2′-0-dimethylinosine); m3C (3-methylcytidine); Cm (2T-0-methylcytidine); s2C (2-thiocytidine); ac4C (N4-acetylcytidine); f5C (5-fonnylcytidine); m5Cm (5,2-0-dimethylcytidine); ac4Cm (N4acetyl2TOmethylcytidine); k2C (lysidine); mIG (1-methylguanosine); m2G (N2-methylguanosine); m7G (7-methylguanosine); Gm (2′-0-methylguanosine); m22G (N2,N2-dimethylguanosine); m2Gm (N2,2′-0-dimethylguanosine); m22Gm (N2,N2,2′-0-trimethylguanosine); Gr(p) (2′-0-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G (archaeosine); D (dihydrouridine); m5Um (5,2′-0-dimethyluridine); s4U (4-thiouridine); m5s2U (5-methyl-2-thiouridine); s2Um (2-thio-2′-0-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-0-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-0-methyluridine); cmnm5U (5-carboxymethylaminomethyluridine); cnmm5Um (5-carboxymethylaminomethyl-2-L-Omethyluridine); cmnm5s2U (5-carboxymethylaminomethyl-2-thiouridine); m62A (N6,N6-dimethyladenosine); Tm (2′-0-methylinosine); m4C (N4-methylcytidine); m4Cm (N4,2-0-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-0-dimethyluridine); m5D (5-methyldihydrouridine); £5Cm (5-formyl-2′-0-methylcytidine); mlGm (1,2′-0-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); and ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(CI-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxycytosine, 5-(CI-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine and 7-deaza-8-substituted purine. For instance, the polynucleotide can include one or more modified pyrimidine nucleobases, such as pseudouridine and/or 5-methylcytosine residues.

The modification in the backbone can include phosphorothioate, phosphoramidate, phosphorodiamidate and phosphorodithioate. At least one or each internucleoside linkage can be a modified internucleoside linkage.

The polynucleotides of the disclosure can include only phosphodiester linkages between nucleosides, but in other examples can contain phosphoramidate, phosphorothioate, phosphorodiamidate and phosphorodithioate and/or methylphosphonate linkages.

Modifications can include N1-methylpseudouridine.

Pseudouridine, N1-methylpseudouridine, 5-methylcytosine, and 5-methoxyuridine can be used in the nucleoside triphosphate pool instead of uridine for incorporation using the polymerase.

As used herein, the term “gapmer” means an oligonucleotide having an internal “centre region” flanked by two external “wing regions” (5′-wing and 3′-wing), wherein the centre region comprises a plurality of nucleotides that support RNase H cleavage and each wing region comprises one or more nucleotides that are chemically distinct to the nucleotides within the centre region. The gapmer is an antisense polynucleotide. The gapmer comprises a centre region, a 5′ wing region positioned at the 5′ end of the centre region, and a 3′ wing region positioned at the 3′ end of the centre region.

As used herein, the term “support material” means a high molecular weight compound or material that increases the molecular weight of a polynucleotide or an oligonucleotide, e.g. the template or primer, thereby allowing it to be retained, e.g. when the impurities and/or products are separated from the reaction mixture.

As used herein “percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein. The query sequence may be 100% identical to the subject sequence, or it may include up to a certain integer number of nucleotide alterations as compared to the subject sequence such that the % identity is less than 100%. For example, the query sequence is at least: 80, 85, 90, 95, 96, 97, 98, or 99% identical to the subject sequence.

As used herein, the term “about” when referring to a measurable value such as an amount, a temporal duration, and the like, is meant to encompass variations of ±20% or ±10%, including f5%, f1%, and f0.1% from the specified value, as such variations that are appropriate to perform the embodiments disclosed herein.

### Embodiments

The disclosure provides a process for producing a single-stranded polynucleotide product having at least one modified nucleotide residue, the process comprising:
a) contacting a template polynucleotide, which comprises a sequence complementary to the single-stranded polynucleotide product, with a pool of at least two segment polynucleotides under conditions to allow annealing of the at least two segment polynucleotides to the template polynucleotide to generate a template polynucleotide with at least two segment polynucleotides annealed thereto, wherein at least one sequence gap is formed between the at least two annealed segment polynucleotides;
b) extending at least one of the annealed segment polynucleotides using a pool of nucleoside triphosphates and a polymerase, to fill in the at least one sequence gap to generate at least one extended segment polynucleotide; and
c) ligating segment polynucleotide(s) and/or extended segment polynucleotide(s) using a ligase to form the single-stranded polynucleotide product bound to the template polynucleotide in a duplex; and
d) changing the conditions to denature the duplex comprising the single-stranded polynucleotide product and the template polynucleotide, whereby the single-stranded polynucleotide product is produced and, optionally, separating the single-stranded polynucleotide product.

Steps (b) and (c) may occur concurrently. In certain situations, steps (a), (b) and (c) may occur concurrently. In an embodiment, the polymerase or the pool of nucleoside triphosphates recited in step b) are present during step (a). In an embodiment, the polymerase recited in step b) is present during step (a) and once the at least two segment polynucleotides have annealed to the template polynucleotide the pool of nucleoside triphosphates is added to enable the polymerase to extend at least one of the annealed segment polynucleotides and fill in the at least one sequence gap. In an alternative embodiment, the pool of nucleoside triphosphates recited in step b) is present during step (a) and once the at least two segment polynucleotides have annealed to the template polynucleotide, the polymerase is added to extend at least one of the annealed segment polynucleotides and fill in the at least one sequence gap.

Step d) may further comprise changing the conditions to denature a duplex comprising an impurity polynucleotide and a template polynucleotide, and separating any impurity polynucleotide(s), prior to changing the conditions to denature the duplex comprising the single-stranded polynucleotide product and template polynucleotide.

In an embodiment, at least one segment polynucleotide comprises at least one modified nucleotide residue. In an embodiment, the pool of nucleoside triphosphates consists of: (i) naturally occurring nucleoside triphosphates; (ii) modified nucleoside triphosphates or (iii) naturally occurring nucleoside triphosphates and modified nucleoside triphosphates.

The disclosure provides a process for producing a single-stranded polynucleotide product having at least one modified nucleotide residue, the process comprising:
a) providing a template polynucleotide comprising a sequence complementary to the single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two segment polynucleotides;
d) providing a pool of nucleoside triphosphates and a polymerase, and extending at least one annealed segment polynucleotide using the pool of nucleoside triphosphates and the polymerase, to fill in the at least one sequence gap;
e) ligating segment polynucleotides and/or extended segment polynucleotides using a ligase to form the single-stranded polynucleotide product;
f) changing the conditions to denature the annealed template and the single-stranded polynucleotide product, and, optionally, separating the single-stranded polynucleotide product.

The disclosure provides a process for producing a single-stranded polynucleotide product having at least one modified nucleotide residue, the process comprising:
a) providing a template polynucleotide comprising a sequence complementary to the single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two segment polynucleotides;
d) providing a pool of nucleoside triphosphates and a polymerase, and extending at least one annealed segment polynucleotide using the pool of nucleoside triphosphates and the polymerase, to fill in the at least one sequence gap;
e) ligating segment polynucleotides and/or extended segment polynucleotides using a ligase to form the single-stranded polynucleotide product;
f) changing the conditions to denature the annealed template and the single-stranded polynucleotide product, and, optionally, separating the single-stranded polynucleotide product,
wherein at least one of the at least two segment polynucleotides comprises at least one modified nucleotide residue and/or the pool of nucleoside triphosphates comprises at least one modified nucleotide.

The disclosure provides a process for producing a single-stranded polynucleotide product having at least one modified nucleotide residue, the process comprising:
a) providing a template polynucleotide comprising a sequence complementary to the single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two segment polynucleotides;
d) providing a pool of nucleoside triphosphates and a polymerase, and extending at least one annealed segment polynucleotide using the pool of nucleoside triphosphates and the polymerase, to fill in the at least one sequence gap;
e) ligating segment polynucleotides and/or extended segment polynucleotides using a ligase to form the single-stranded polynucleotide product;
f) optionally, changing the conditions to denature the annealed template and any impurities, and separating the impurities; and
g) changing the conditions to denature the annealed template and the single-stranded polynucleotide product, and, optionally, separating the single-stranded polynucleotide product.

The disclosure provides a process for producing a single-stranded polynucleotide product having at least one modified nucleotide residue, the process comprising:
a) providing a template polynucleotide comprising a sequence complementary to the single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two segment polynucleotides;
d) providing a pool of nucleoside triphosphates and a polymerase, and extending at least one annealed segment polynucleotide using the pool of nucleoside triphosphates and the polymerase, to fill in the at least one sequence gap;
e) ligating segment polynucleotides and/or extended segment polynucleotides using a ligase to form the single-stranded polynucleotide product;
f) optionally, changing the conditions to denature the annealed template and any impurities, and separating the impurities; and
g) changing the conditions to denature the annealed template and the single-stranded polynucleotide product, and, optionally, separating the single-stranded polynucleotide product,
wherein at least one of the at least two segment polynucleotides comprises at least one modified nucleotide residue and/or the pool of nucleoside triphosphates comprises at least one modified nucleotide.

The disclosure provides a process for producing a single-stranded polynucleotide product having at least one modified nucleotide residue, the process comprising:
a) providing a template polynucleotide comprising a sequence complementary to the single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two segment polynucleotides;
d) providing a pool of nucleoside triphosphates and a polymerase, and extending at least one annealed segment polynucleotide using the pool of nucleoside triphosphates and the polymerase, to fill in the at least one sequence gap;
e) ligating segment polynucleotides and/or extended segment polynucleotides using a ligase to form the single-stranded polynucleotide product;
f) changing the conditions to denature the annealed template and any impurities, and separating the impurities; and
g) changing the conditions to denature the annealed template and the single-stranded polynucleotide product, and, optionally, separating the single-stranded polynucleotide product.

The disclosure provides a process for producing a single-stranded polynucleotide product having at least one modified nucleotide residue, the process comprising:
a) providing a template polynucleotide comprising a sequence complementary to the single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two segment polynucleotides;
d) providing a pool of nucleoside triphosphates and a polymerase, and extending at least one annealed segment polynucleotide using the pool of nucleoside triphosphates and the polymerase, to fill in the at least one sequence gap;
e) ligating segment polynucleotides and/or extended segment polynucleotides using a ligase to form the single-stranded polynucleotide product;
f) changing the conditions to denature the annealed template and any impurities, and separating the impurities; and
g) changing the conditions to denature the annealed template and the single-stranded polynucleotide product, and, optionally, separating the single-stranded polynucleotide product,
wherein at least one of the at least two segment polynucleotides comprises at least one modified nucleotide residue and/or the pool of nucleoside triphosphates comprises at least one modified nucleotide.

The present disclosure provides a process for producing a single-stranded polynucleotide product having at least one modified nucleotide residue, the process comprising:
a) providing a template polynucleotide comprising a sequence complementary to the single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two segment polynucleotides;
d) providing a pool of nucleoside triphosphates and a polymerase, and extending at least one annealed segment polynucleotide using the pool of nucleoside triphosphates and the polymerase, to fill in the at least one sequence gap;
e) ligating segment polynucleotides and/or extended segment polynucleotides using a ligase to form the single-stranded polynucleotide product;
f) optionally, changing the conditions to denature the annealed template and any impurities, and separating the impurities; and
g) changing the conditions to denature the annealed template and the single-stranded polynucleotide product and, optionally, separating the single-stranded polynucleotide product.

The pool of nucleoside triphosphates may comprise at least one modified nucleoside triphosphate.

Step d) may be further divided into three separate steps, namely d1) providing a pool of nucleoside triphosphates, d2) providing a polymerase and d3) extending at least one annealed segment polynucleotide using the pool of nucleoside triphosphates and the polymerase, to fill in the at least one sequence gap.

The present disclosure provides a process for producing a single-stranded polynucleotide product having at least one modified nucleotide residue, the process comprising:
a) providing a template polynucleotide comprising a sequence complementary to the single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two annealed segment polynucleotides;
d) providing a pool of nucleoside triphosphates;
e) providing a polymerase;
f) extending at least one annealed segment polynucleotide using the pool of nucleoside triphosphates and the polymerase, to fill in the at least one sequence gap;
g) ligating segment polynucleotides and/or extended segment polynucleotides using a ligase to form the single-stranded polynucleotide product;
h) optionally, changing the conditions to denature the annealed template and any impurities, and separating the impurities; and
i) changing the conditions to denature the annealed template and the single-stranded polynucleotide product and, optionally, separating the single-stranded polynucleotide product.

Two or more of steps a) to i) may occur concurrently, optionally steps f) and g) may occur concurrently. Two or more of steps a) to i) may occur sequentially, optionally steps f) and g) may occur sequentially.

In any of the above-described processes, the process may additionally comprise a further step (step j) of recycling the template. The process may additionally comprise another step (step k) of repeating the previous steps (steps a) to i) or steps a) to j)) with the recycled template.

The present disclosure also provides a process for producing a single-stranded polynucleotide product having at least one modified nucleotide residue, the process comprising:
a) providing a template polynucleotide that comprises a sequence complementary to the single-stranded polynucleotide product and:
   i) a hairpin loop that acts as a primer for a polymerase,
   ii) a hairpin loop that acts as a stopper for a polymerase, or
   iii) hairpin loops at both ends of the template polynucleotide, with one hairpin loop acting as a primer for a polymerase and the other hairpin loop active as a stopper for the polymerase;
b) contacting the template polynucleotide with a pool of at least one segment polynucleotide under conditions to allow annealing of the at least one segment polynucleotide to the template polynucleotide to generate a template polynucleotide with the at least one segment polynucleotide annealed thereto, wherein at least one sequence gap is formed between the annealed segment polynucleotide and the end of the hairpin loop;
c) extending the annealed segment polynucleotide or the end of the hairpin loop using a pool of nucleoside triphosphates and a polymerase, to fill in the at least one sequence gap to generate at least one extended segment polynucleotide or extended hairpin polynucleotide;
d) ligating segment polynucleotide(s) and/or extended segment/hairpin polynucleotide(s) using a ligase to form the single-stranded polynucleotide product bound to the template polynucleotide in a duplex;
e) cleaving the single-stranded polynucleotide product from the template polynucleotide; and
f) changing the conditions to denature the duplex comprising the single-stranded polynucleotide product and the template polynucleotide, whereby the single-stranded polynucleotide product is produced. The cleavage step may be carried out by a nuclease, a nickase, a DNAzyme, or by a chemical method.

The present disclosure also provides a process for producing a double-stranded polynucleotide product, the process comprising annealing two complementary single-stranded polynucleotide products, at least one of which has been produced by the process of the present disclosure, optionally wherein both of which have been produced by the process of the present disclosure.

The disclosure also provides a process for producing a double-stranded polynucleotide product having at least one modified nucleotide residue, the process comprising:
a) contacting a template polynucleotide, which comprises a sequence complementary to a single-stranded polynucleotide product, with a pool of at least two segment polynucleotides under conditions to allow annealing of the at least two segment polynucleotides to the template polynucleotide to generate a template polynucleotide with the at least two segment polynucleotides annealed thereto, wherein at least one sequence gap is formed between the at least two annealed segment polynucleotides;
b) extending at least one of the annealed segment polynucleotides using a pool of nucleoside triphosphates and a polymerase, to fill in the at least one sequence gap to generate at least one extended segment polynucleotide;
c) ligating segment polynucleotide(s) and/or extended segment polynucleotide(s) using a ligase to form the single-stranded polynucleotide product bound to the template polynucleotide in a duplex;
d) optionally, changing the conditions to denature a duplex comprising the single-stranded template polynucleotide and an impurity polynucleotide, and separating any impurity polynucleotide(s) from the template polynucleotide;
e) changing the conditions to denature the duplex comprising the single-stranded polynucleotide product and the template polynucleotide, whereby the single-stranded polynucleotide product is produced; and
f) using the single-stranded polynucleotide product as the template polynucleotide in step a) and repeating steps a) to c) or steps a) to d) to produce the double-stranded polynucleotide product.

Without being bound by theory, these processes have been found to reduce the complexity of the production of the polynucleotide or oligonucleotide product by allowing the steps of segment polynucleotide and/or oligonucleotide production and the ligation method steps to use the same template. Concurrent segment polynucleotide and/or oligonucleotide extension and ligation can further occur in producing the polynucleotide and/or oligonucleotide product. These processes can assist with controlling the chirality of the final product and reduce the number of ligation steps, reducing the overall reaction time.

The disclosure also provides a process for producing a double-stranded polynucleotide or oligonucleotide product, wherein two complementary single-stranded polynucleotides or oligonucleotides produced by the process of the present disclosure are mixed under conditions to allow annealing.

Alternatively, the disclosure also provides a process for producing a double-stranded polynucleotide or oligonucleotide product, wherein the single-stranded polynucleotide or oligonucleotide product acts and/or is used as a template.

For example, the disclosure provides a process for producing a double-stranded polynucleotide product having at least one modified nucleotide residue, the process comprising:
a) providing a template polynucleotide comprising a sequence complementary to a single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two annealed segment polynucleotides;
d) providing a pool of nucleoside triphosphates and a polymerase, and extending at least one annealed segment polynucleotide using the pool of nucleoside triphosphates and the polymerase, to fill in the at least one sequence gap;
e) ligating segment polynucleotides and/or extended segment polynucleotides using a ligase to form the single-stranded polynucleotide product;
f) optionally, changing the conditions to denature the annealed template and any impurities, and separating the impurities;
g) changing the conditions to denature the annealed template and the single-stranded polynucleotide product and, optionally, separating the single-stranded polynucleotide product; and
h) using the single-stranded polynucleotide product as the template in step a) and repeating steps a) to e) or steps a) to f) to produce the double-stranded polynucleotide product.

Optionally, the double-stranded polynucleotide product is purified.

For example, the disclosure provides a process for producing a double-stranded polynucleotide product having at least one modified nucleotide residue, the process comprising:
a) providing a template polynucleotide comprising a sequence complementary to a single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two annealed segment polynucleotides;
d) providing a pool of nucleoside triphosphates and a polymerase, and extending at least one segment polynucleotide using the pool of nucleoside triphosphates and the polymerase, to fill in the at least one sequence gap;
e) ligating segment polynucleotides and/or extended segment polynucleotides using a ligase to form the single-stranded polynucleotide product;
f) optionally, changing the conditions to denature the annealed template and any impurities, and separating the impurities;
g) changing the conditions to denature the annealed template and the single-stranded polynucleotide product and, optionally, separating the single-stranded polynucleotide product; and
h) using the single-stranded polynucleotide product as the template in step a) and repeating steps a) to e) or steps a) to f) to produce the double-stranded polynucleotide product,
wherein at least one segment polynucleotide comprises at least one modified nucleotide residue and/or the pool of nucleoside triphosphates comprises at least one modified nucleotide.

Optionally, the double-stranded polynucleotide product is purified.

In some embodiments, at least one segment polynucleotide comprises at least one modified nucleotide residue. In some embodiments, at least two segment polynucleotides comprise at least one modified nucleotide residue. In some embodiments, all segment polynucleotides comprise at least one modified nucleotide residue.

In certain embodiments, at least one nucleoside triphosphate in the pool of nucleoside triphosphates in step (d) is modified. In certain embodiments, A, T, C, G and/or U in the pool of nucleoside triphosphates in step (d) is modified. In certain embodiments, all nucleoside triphosphates in the pool of nucleoside triphosphates in step (d) are modified.

In some embodiments, the ligase used in step I is used to ligate the 3′ and/or 5′ end of each segment polynucleotide or extended segment polynucleotide to each adjacent segment polynucleotide or extended segment polynucleotide to form a product polynucleotide strand. The ligase used in step (e) can be an RNA and/or a DNA ligase.

In some embodiments, the pool of polynucleotides is produced by enzymatic synthesis, chemical synthesis, optionally solid supported synthesis or solution phase synthesis, or a combination thereof. The pool of polynucleotides can be produced by enzymatic synthesis using a single-stranded ligase, a transferase, a polymerase or a combination thereof.

In some embodiments, the modification is selected from the group consisting of a modified sugar moiety, modification of the nucleobase and modification of the backbone. In some embodiments, the at least one modified nucleotide comprises modification of the sugar moiety, modification of the nucleobase and/or modification of the backbone. In other words, the at least one modified nucleotide comprises a modified sugar moiety, a modified nucleobase and/or a modified backbone.

Polynucleotides or oligonucleotides used in the process of the invention may include sugar modifications, i.e. a modified version of the ribosyl moiety, such as 2′-O-modified RNA such as 2′-O-alkyl or 2′-O-(substituted)alkyl e.g. 2′-O-methyl, 2′-O-(2-cyanoethyl), 2′-O-(2-methoxy)ethyl (2′-MOE), 2′-O-(2-thiomethyl)ethyl, 2′-O-butyryl, 2′-O-propargyl, 2′-O-allyl, 2′-O-(3-amino)propyl, 2′-O-(3-(dimethylamino)propyl), 2′-O-(2-amino)ethyl, 2′-O-(2-(dimethylamino)ethyl); 2′-deoxy (DNA); 2′-O-(haloalkoxy)methyl (Arai K. et al. Bioorg. Med. Chem. 2011, 21, 6285) e.g. 2′-O-(2-chloroethoxy)methyl (MCEM), 2′-O-(2, 2-dichloroethoxy)methyl (DCEM); 2′-O-alkoxycarbonyl e.g. 2′-O-[2-(methoxycarbonyl)ethyl](MOCE), 2′-O-[2-(N-methylcarbamoyl)ethyl](MCE), 2′-O-[2-(N, N-dimethylcarbamoyl)ethyl](DCME); 2′-halo e.g. 2′-F, FANA (2′-F arabinosyl nucleic acid); carbasugar and azasugar modifications; 3-O-alkyl e.g. 3-O-methyl, 3′-O-butyryl, 3-0-propargyl; and their derivatives.

In an embodiment of the invention, the sugar modification is selected from the group consisting of 2′-Fluoro (2′-F), 2′-O-methyl (2′-OMe), 2′-O-methoxyethyl (2′-MOE), and 2′-amino. In a yet further embodiment, the modification is 2′-MOE.

Other sugar modifications include “bridged” or “bicyclic” nucleic acid (BNA), e.g. locked nucleic acid (LNA), xylo-LNA, α-L-LNA, β-D-LNA, cEt (2′-O,4′-C constrained ethyl) LNA, cMOEt (2′-O,4′-C constrained methoxyethyl) LNA, ethylene-bridged nucleic acid (ENA), tricyclo DNA; unlocked nucleic acid (UNA); cyclohexenyl nucleic acid (CeNA), altriol nucleic acid (ANA), hexitol nucleic acid (HNA), fluorinated HNA (F-HNA), pyranosyl-RNA (p-RNA), 3′-deoxypyranosyl-DNA (p-DNA); morpholino (as e.g. in PMO, PPMO, PMOPlus, PMO-X); and their derivatives.

Polynucleotides or oligonucleotides used in the process of the disclosure may include other modifications, such as peptide-base nucleic acid (PNA), boron modified PNA, pyrrolidine-based oxy-peptide nucleic acid (POPNA), glycol- or glycerol-based nucleic acid (GNA), threose-based nucleic acid (TNA), acyclic threoninol-based nucleic acid (aTNA), oligonucleotides with integrated bases and backbones (ONIBs), pyrrolidine-amide oligonucleotides (POMs); and their derivatives.

In an embodiment of the invention, the modified oligonucleotide comprises a phosphorodiamidate morpholino oligomer (PMO), a locked nucleic acid (LNA), a peptide nucleic acid (PNA), a bridged nucleic acid (BNA) such as (S)-cEt-BNA, or a L-Ribonucleic acid also known as SPIEGELMER.

In a further embodiment, the modification is in the nucleobase. Base modifications include modified versions of the natural purine and pyrimidine bases (e.g. adenine, uracil, guanine, cytosine, and thymine), such as inosine, hypoxanthine, orotic acid, agmatidine, lysidine, 2-thiopyrimidine (e.g. 2-thiouracil, 2-thiothymine), G-clamp and its derivatives, 5-substituted pyrimidine (e.g. 5-methylcytosine, 5-methyluracil, 5-halouracil, 5-propynyluracil, 5-propynylcytosine, 5-aminomethyluracil, 5-hydroxymethyluracil, 5-aminomethylcytosine, 5-hydroxymethylcytosine, Super T), 2,6-diaminopurine, 7-deazaguanine, 7-deazaadenine, 7-aza-2, 6-diaminopurine, 8-aza-7-deazaguanine, 8-aza-7-deazaadenine, 8-aza-7-deaza-2, 6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), or derivatives thereof; and degenerate or universal bases, like 2, 6-difluorotoluene or absent bases like abasic sites (e.g. 1-deoxyribose, 1,2-dideoxyribose, 1-deoxy-2-0-methylribose; or pyrrolidine derivatives in which the ring oxygen has been replaced with nitrogen (azaribose)). Examples of derivatives of Super A, Super G and Super T can be found in U.S. Pat. No. 6,683,173. cPent-G, cPent-AP and Pr-AP were shown to reduce immunostimulatory effects when incorporated in siRNA (Peacock H. et al. J. Am. Chem. Soc. (2011), 133, 9200).

In an embodiment of the invention, the nucleobase modification is selected from the group consisting of 5-methyl pyrimidines, 7-deazaguanosines and abasic nucleotides. In an embodiment, the modification is a 5-methyl cytosine.

Polynucleotides or oligonucleotides used in the process of this disclosure may include a backbone modification, e.g. a modified version of the phosphodiester present in RNA, such as phosphorothioate (PS), phosphorodithioate (PS2), phosphonoacetate (PACE), phosphonoacetamide (PACA), thiophosphonoacetate, thiophosphonoacetamide, phosphorothioate prodrug, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methylboranophosphonothioate, and their derivatives. Other modifications include phosphoramidite, phosphoramidate, ′3′→P5′ phosphoramidate, phosphordiamidate, phosphorothiodiamidate, sulfamate, dimethylenesulfoxide, sulfonate, triazole, oxalyl, carbamate, methyleneimino (MMI), and thioacetamido nucleic acid (TANA); and their derivatives.

In a further embodiment, the modification is in the backbone and is selected from the group consisting of: phosphorothioate (PS), phosphoramidate (PA), phosphorodiamidate and phosphorodithioate (PS2). In an embodiment of the invention, the modified oligonucleotide is a phosphorodiamidate morpholino oligomer (PMO). A PMO has a backbone of methylenemorpholine rings with phosphorodiamidate linkages. In an embodiment of the invention the product has a phosphorothioate (PS) backbone. In an embodiment of the invention, the product has at least one phosphorothioate (PS) bond in the backbone.

In an embodiment of the invention, the oligonucleotide comprises a combination of two or more modifications as disclosed herein. A person skilled in the art will appreciate that there are many synthetic derivatives of oligonucleotides and their constituent nucleotides.

The modification can comprise a modification at the 2′ position of the sugar moiety, a bicyclic sugar or a 4′-CH(CH₃)—O-2′ group, and combinations thereof. The modification at the 2′ position of the sugar moiety can comprise a 2′-MOE. The modified nucleobase can comprise a cytosine, optionally a 5-methyl cytosine. The modification in the nucleobase can be selected from the group comprising a 5-methyl pyrimidine, a 7-deazaguanosine and an abasic nucleotide. The modification in the backbone can be selected from the group comprising phosphorothioate, phosphoramidate, phosphorodiamidate and phosphodithioate.

In embodiments, each polynucleotide segment can be comprised of linked 2′-deoxynucleotides or deoxynucleosides. In embodiments, at least one or each internucleoside linkage can be a modified internucleoside linkage.

In embodiments, the single-stranded polynucleotide product can be a DNA polynucleotide product, an RNA polynucleotide product, or a combination thereof. The RNA polynucleotide product can be an mRNA. In embodiments, the nucleotide modification can comprise substitution of one or more uracil residues. The at least one modified nucleotide may comprise 1-methyl-pseudouridine, 5-methoxy-uracil, 1-ethyl-pseudouracil, pseudouracil, 1-methylpseudouracil, 5-methyl-cytidine, 5-methyl-cytosine, N6-methyladenosine or 7-methylguanosine, or combinations thereof. In an embodiment, the single-stranded polynucleotide product is a mRNA wherein each U residue is N1-methyl-pseudouridine. In an embodiment, the single-stranded polynucleotide product is a mRNA wherein each U residue is N1-methyl-pseudouridine and the backbone is a phosphorothioate backbone.

In some embodiments, the polymerase used in step (d) lacks 5′ to 3′ exonuclease activity. In some embodiments, the polymerase used in step (d) lacks 3′ to 5′ exonuclease activity. In some embodiments, the polymerase used in step (d) lacks 5′ to 3′ exonuclease activity and lacks 3′ to 5′ exonuclease activity.

In embodiments, the polymerase used in step (d) can be a DNA polymerase, an RNA polymerase or a combination thereof.

The steps of the process can be performed in sequential order. The steps of the process can be reordered so long as they do not render the claim unworkable. When providing the pool of nucleoside triphosphates, providing the polymerase, and extending the at least one segment polynucleotide using the pool of nucleoside triphosphates and the polymerase, to fill in the at least one sequence gap, occurs as separate steps, the pool of nucleoside triphosphates can be provided in any preceding step so long as the polymerase is added after the segment polynucleotides or oligonucleotides are annealed to the template. Alternatively, the polymerase can be provided in any preceding step so long as the pool of nucleoside triphosphates is added after the segment polynucleotides or oligonucleotides are annealed to the template. In some embodiments, two or more steps occur concurrently. In some embodiments, two or more of steps a) to g) occur concurrently. In some embodiments, two or more steps occur sequentially. In some embodiments, two or more of steps a) to g) occur sequentially. In some embodiments, steps (d) and (e) of the process occur concurrently. In other embodiments, steps (d) and (e) of the process occur sequentially.

In embodiments, the template polynucleotide or oligonucleotide can consist of a sequence complementary to the sequence of the single-stranded polynucleotide or oligonucleotide product.

In embodiments, the template polynucleotide is part of a hairpin loop. The terms hairpin loop and stem loop may be used interchangeably. The hairpin loop may be asymmetric. The hairpin loop may be a DNA hairpin loop. The hairpin loop may be an RNA hairpin loop. If the template polynucleotide comprises a hairpin loop the polymerase may not require a primer to initiate polymerization. The single-stranded polynucleotide product may be released from the template and hairpin loop using an enzyme to introduce a single-strand cut between the hairpin loop and product polynucleotide strand (e.g. using a nickase) followed by denaturation of the annealed template polynucleotide and product polynucleotide strands.

In embodiments, the single-stranded polynucleotide product can be 3 to 40 nucleotides long, 3 to 35 nucleotides long, or 3 to 30 nucleotides long, optionally 10 to 35 nucleotides long, 10 to 30 nucleotides long, 3 to 15 nucleotides long, 13 to 35 nucleotides long, 15 to 35 nucleotides long, 13 to 30 nucleotides long, 15 to 30 nucleotides long, 13 to 25 nucleotides long, 15 to 25 nucleotides long, 13 to 20 nucleotides long, 15 to 20 nucleotides long, 17 to 25 nucleotides long, 20 to 25 nucleotides long, or 20 to 30 nucleotides long. In an embodiment, the product is 20 nucleotides long and comprises three segment polynucleotides comprising:
(i) a 5′-segment that is 7 nucleotides long, a central segment that is 6 nucleotides long and a 3-segment that is 7 nucleotides long;
(ii) a 5′-segment that is 6 nucleotides long, a central segment that is 8 nucleotides long and a 3-segment that is 6 nucleotides long;
(iii) a 5′-segment that is 5 nucleotides long, a central segment that is 10 nucleotides long and a 3-segment that is 5 nucleotides long;
(iv) a 5′-segment that is 4 nucleotides long, a central segment that is 12 nucleotides long and a 3-segment that is 4 nucleotides long; or
(v) a 5′-segment that is 3 nucleotides long, a central segment that is 14 nucleotides long and a 3-segment that is 3 nucleotides long. The single-stranded polynucleotide product can be a gapmer antisense polynucleotide comprising a centre region, a 5′ wing region positioned at the 5′ end of the centre region, and a 3′ wing region positioned at the 3′ end of the centre region. This is particularly useful in therapeutic oligonucleotide production, where it is important to produce highly sequence specific oligonucleotide products.

In embodiments, the at least two segment polynucleotides comprise:
(i) a 5′-segment that is 7 nucleotides long and a 3-segment that is 7 nucleotides long;
(ii) a 5′-segment that is 6 nucleotides long and a 3-segment that is 6 nucleotides long;
(iii) a 5′-segment that is 5 nucleotides long and a 3-segment that is 5 nucleotides long;
(iv) a 5′-segment that is 4 nucleotides long and a 3-segment that is 4 nucleotides long; or
(v) a 5′-segment that is 3 nucleotides long and a 3-segment that is 3 nucleotides long.

In embodiments, the single-stranded polynucleotide product can be 30 to 20,000 nucleotides long, optionally 30 to 10,000 nucleotides long, 30 to 5,000 nucleotides long, 30 to 4,000 nucleotides long, 30 to 3,000 nucleotides long, 30 to 2,000 nucleotides long, 30 to 1,000 nucleotides long, 30 to 500 nucleotides long, 30 to 400 nucleotides long, 30 to 300 nucleotides long, 30 to 200 nucleotides long, 30 to 100 nucleotides long, 30 to 50 nucleotides long, or 30 to 40 nucleotides long. These products are useful, for example in therapeutic mRNA production, where it is important to produce highly sequence specific polynucleotide products.

In a further embodiment, the single-stranded polynucleotide product is greater than 30 nucleotides in length. In another embodiment, the single-stranded polynucleotide product is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides, or greater than 5000 nucleotides.

In embodiments, the template has a property that allows it to be separated from the product and recycled for future reactions. The process can comprise a final step of recycling the template. The process can comprise step j) recycling the template. The process can comprise a final step of recycling the template for use in future reactions. The process can comprise step j) recycling the template for use in future reactions. The process can comprise a final step of recycling the template and repeating the process. The process can comprise a final step of recycling the template and repeating one or more steps of the process. The process can comprise step j) recycling the template and step k) repeating steps a) to i). The process can comprise step j) recycling the template and step k) repeating steps a) to k)

The processes described herein can be semi-continuous or continuous.

Provided are processes where the product is produced at gram-scale or kilogram-scale, or greater scale and/or the processes are carried out in a reaction volume of at least 1 L. Provided are processes where the product is produced at gram-scale, kilogram-scale, or greater scale. Provided are processes wherein the processes are carried out in a reaction volume of at least 200 mL. Provided are processes wherein the processes are carried out in a reaction volume of at least 500 mL. Provided are processes wherein the processes are carried out in a reaction volume of at least 1 L. Provided are processes wherein the processes are carried out in a reaction volume of at least 2 L. Provided are processes wherein the processes are carried out in a reaction volume of at least 5 L.

The resulting single-stranded polynucleotide or oligonucleotide product can be at least 90% pure, at least 95% pure, or at least 98% pure.

The process can be used to produce a therapeutic polynucleotide or oligonucleotide. In embodiments, the processes are processes for producing a single-stranded therapeutic polynucleotide or oligonucleotide. In embodiments, the processes are processes for producing a double-stranded therapeutic polynucleotide or oligonucleotide. These processes are useful, for example, in therapeutic oligonucleotide production, where it is important to produce highly sequence specific oligonucleotide products. The products are also useful, for example in therapeutic mRNA production, where it is important to produce highly sequence specific polynucleotide products.

Provided are processes whereby denaturing the template and impurity duplex and/or denaturing the template and product duplex results from a temperature increase. In embodiments, denaturation can occur as a result of changing the pH. In embodiments, denaturation can occur by changing the salt concentration in a buffering solution.

Provided are processes whereby the segment oligonucleotides are 3 to 15 nucleotides long. In embodiments, the segments can be 5 to 10 nucleotides long. In embodiments, the segments can be 5 to 8 nucleotides long. In embodiments, the segments can be 4, 5, 6, 7 or 8 nucleotides long. In embodiments, there are three segment oligonucleotides: a 5′-segment that is 7 nucleotides long, a central segment that is 6 nucleotides long and a 3′-segment that is 7 nucleotides long, which when ligated together form an oligonucleotide that is 20 nucleotides long (a “20-mer”). In embodiments, there are three segment oligonucleotides: a 5′-segment that is 6 nucleotides long, a central segment that is 8 nucleotides long and a 3′-segment that is 6 nucleotides long, which when ligated together form an oligonucleotide that is 20 nucleotides long (a “20-mer”). In embodiments, there are three segment oligonucleotides: a 5′-segment that is 5 nucleotides long, a central segment that is 10 nucleotides long and a 3′-segment that is 5 nucleotides long, which when ligated together form an oligonucleotide that is 20 nucleotides long (a “20-mer”). In embodiments, there are three segment oligonucleotides: a 5′-segment that is 4 nucleotides long, a central segment that is 12 nucleotides long and a 3′-segment that is 4 nucleotides long, which when ligated together form an oligonucleotide that is 20 nucleotides long (a “20-mer”). In embodiments, there are four segment oligonucleotides: a 5′-segment that is 5 nucleotides long, a 5′-central segment that is 5 nucleotides long, a central-3′-segment that is 5 nucleotides long, and a 3′-segment that is 5 nucleotides long, which when ligated together form an oligonucleotide that is 20 nucleotides long (a “20-mer”).

Provided are processes whereby the product is 3 to 40 nucleotides long. In embodiments, the product can be 13 to 40 nucleotides long. In embodiments, the product can be 15 to 40 nucleotides long. In embodiments, the product can be 13 to 35 nucleotides long. In embodiments, the product can be 15 to 35 nucleotides long. In embodiments, the product can be 15 to 30 nucleotides long. In embodiments, the product can be 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides long. In an embodiment of the invention, the product is 20 nucleotides long, a “20-mer”. In an embodiment of the invention, the product is 21 nucleotides long, a “21-mer”. In an embodiment of the invention, the product is 22 nucleotides long, a “22-mer”. In an embodiment of the invention, the product is 23 nucleotides long, a “23-mer”. In an embodiment of the invention, the product is 24 nucleotides long, a “24-mer”. In an embodiment of the invention, the product is 25 nucleotides long, a “25-mer”. In an embodiment of the invention, the product is 26 nucleotides long, a “26-mer”. In an embodiment of the invention, the product is 27 nucleotides long, a “27-mer”. In an embodiment of the invention, the product is 28 nucleotides long, a “28-mer”. In an embodiment of the invention, the product is 29 nucleotides long, a “29-mer”. In an embodiment of the invention, the product is 30 nucleotides long, a “30-mer”. Such products have utility, for example, in gapmer production. In an embodiment, such 3 to 40 nucleotides long single-stranded products are therapeutic antisense gapmers. In an embodiment, such 3 to 40 nucleotides long products are therapeutic double-stranded products, such as siRNAs and miRNAs. In an embodiment, such 3 to 40 nucleotides long products are oligonucleotides that recruit and guide DNA and/or RNA editing enzymes, for example RNA base-modifying oligonucleotides, such as AImers.

Provided are processes whereby the product is up to 10,000 nucleotides long. In embodiments, the single-stranded polynucleotide product can be 10 to 10,000 nucleotides long, optionally 10 to 5,000 nucleotides long, 10 to 1,000 nucleotides long, 10 to 500 nucleotides long, 10 to 400 nucleotides long, 10 to 300 nucleotides long, 10 to 200 nucleotides long, 10 to 100 nucleotides long, 10 to 50 nucleotides long, or 10 to 40 nucleotides long. Such products have utility, for example, as therapeutic mRNA polynucleotides.

Provided are processes wherein the property that allows the template to be separated from the product is that the template is attached to a support material. In embodiments, the support material is a soluble support material. In embodiments, the soluble support material is selected from the group consisting of: polyethylene glycol, a soluble organic polymer, DNA, a protein, a dendrimer, a polysaccharide, an oligosaccharide, and a carbohydrate. In embodiments, the support material is polyethylene glycol (PEG). In embodiments, the support material is an insoluble support material. In embodiments, the support material is a solid support material. In embodiments, the solid support material is selected from the group consisting of: a glass bead, a polymeric bead, a fibrous support, a membrane, a streptavidin coated bead and cellulose. In embodiments, the solid support material is a streptavidin coated bead. In embodiments, the solid support material is part of the reaction vessel itself, e.g. a reaction wall.

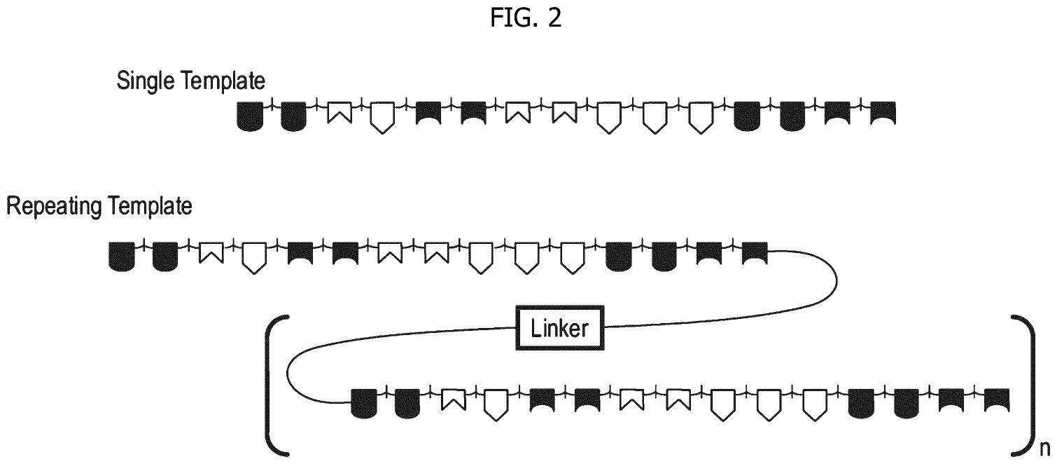

Provided are processes wherein repeated copies of the template are attached in a continuous manner via a single attachment point to the support material. The repeated copies of the template may be separated by a linker, e.g. as shown in FIG. 2. The repeated copies of the template may be direct repeats, i.e. they are not separated by a linker.

In embodiments, the template is attached to the support material at multiple attachment points.

Provided are processes wherein the property that allows the template to be separated from the product is the molecular weight of the template. For example, repeated copies of the template sequence may be present on a single polynucleotide or oligonucleotide, with or without a linker sequence.

Provided are processes wherein the template, or the template and support material, are recycled for use in future reactions, for example as detailed below. Provided are processes wherein the reaction is carried out using a continuous or semi-continuous flow process.

In embodiments, the processes are for large-scale manufacture of polynucleotides or oligonucleotides, optionally therapeutic polynucleotides or oligonucleotides. In the context of the present disclosure, large-scale manufacture of polynucleotides or oligonucleotides means manufacture at a scale greater than or equal to 1 litre, e.g. the process is carried out in a 1 L or larger reactor. Alternatively, or in addition, in the context of the present disclosure large-scale manufacture of polynucleotides or oligonucleotides means manufacture at gram-scale of product, in particular the production of greater than or equal to 10 grams of product. In embodiments, the product is produced at gram-scale or kilogram-scale and/or the processes are carried out in a reactor of at least 1 L. In embodiments, the amount of polynucleotide or oligonucleotide product produced is at gram-scale. In an embodiment of the disclosure the amount of product produced is greater than or equal to: 1, 2, 3, 4, 5, 6, 7, 8, 9, 10, 20, 30, 40, 50, 60, 70, 80, 90 or 100 grams. In embodiments, the amount of oligonucleotide product produced is greater than or equal to: 150, 200, 250, 300, 350, 400, 450, 500, 550, 600, 650, 700, 750, 800, 850, 900, 950 grams. In embodiments, the amount of polynucleotide or oligonucleotide product produced is 500 grams or greater. In embodiments, the polynucleotide or oligonucleotide product produced is at kilogram-scale. In embodiments, the amount of polynucleotide or oligonucleotide product produced is 1 kg or more. In embodiments, the amount of polynucleotide or oligonucleotide product produced is greater than or equal to: 1, 2, 3, 4, 5, 6, 7, 8, 9, 10 kg. In embodiments, the amount of polynucleotide or oligonucleotide product produced is greater than or equal to: 15, 20, 25, 30, 35, 40, 45, 50, 55, 60, 65, 70, 75, 80, 85, 90, 95 or 100 kg.

In embodiments, the amount of product produced is between 10 grams and 100 kg. In embodiments, the amount of product produced is between 10 grams and 50 kg. In embodiments, the amount of product produced is between 100 grams and 100 kg. In embodiments, the amount of product produced is between 100 grams and 50 kg. In embodiments, the amount of product produced is between 500 grams and 100 kg. In embodiments, the amount of product produced is between 500 grams and 50 kg. In embodiments, the amount of product produced is between 1 kg and 50 kg. In embodiments, the amount of product produced is between 10 kg and 50 kg.

In embodiments, polynucleotide or oligonucleotide manufacture takes place at a scale greater than or equal to: 2, 3, 4, 5, 6, 7, 8, 9, 10 litres, e.g. in a 2, 3, 4, 5, 6, 7, 8, 9 or 10 L reactor. In embodiments, polynucleotide or oligonucleotide manufacture takes place at a scale greater than or equal to: 20, 25, 30, 35, 40, 45, 50, 55, 60, 65 70, 75, 80, 85, 90, 95, 100 litres, e.g. in a 20, 25, 30, 35, 40, 45, 50, 55, 60, 65 70, 75, 80, 85, 90, 95, 100 L reactor. In embodiments, polynucleotide or oligonucleotide manufacture takes place at a scale greater than or equal to: 150, 200, 250, 300, 350, 400, 450, 500, 550, 600, 650, 700, 750, 800, 850, 900, 950, 1000 litres, e.g. in 150, 200, 250, 300, 350, 400, 450, 500, 550, 600, 650, 700, 750, 800, 850, 900, 950, 1000 L reactor.

In embodiments, the reactor volume is about 10,000 L, about 5000 L, about 2000 L, about 1000 L, about 500 L, about 125 L, about 50 L, about 20 L, about 10 L, or about 5 L.

In embodiments, the reactor volume is between 5 and 10,000 L, between 10 and 5000 L, between 20 and 2000 L, or between 50 and 1000 L.

Provided are processes for producing polynucleotides using a polymerase and ligase. A template polynucleotide is used to which two or more complementary polynucleotide or oligonucleotide sequences, e.g. segments, are bound. A polymerase is used to fill in each gap between the complementary polynucleotide or oligonucleotide sequences. Adjacent sequences are fused together using a ligase. Advantageously, the overall process can be fully enzymatic, reducing the complexity and increasing the efficiency of the process. The two or more complementary polynucleotide or oligonucleotide sequences can include modified nucleotides. Alternatively, or additionally, the nucleotides introduced by the polymerase can include modified nucleotides. This allows the production of therapeutic polynucleotides and oligonucleotides, which can be short oligonucleotides (e.g. 10 to 30 nucleotides in length) or long polynucleotides (e.g. 30 to 10,000 nucleotides in length). Two or more complementary polynucleotide or oligonucleotide sequences can be bound to the template polynucleotide and the polymerase can fill in each gap between each complementary polynucleotide or oligonucleotide sequence. A ligase can then be used to ligate each adjacent sequence together.

The final single-stranded product can be separated from the template using the methods described herein. Optionally, the template can have a property as described herein which allows the template to be recycled for future reactions.

In one example, provided are processes for producing a gapmer, which is an oligonucleotide, usually with a length of 10 to 30 nucleotides, containing at least one modified nucleotide residue. Generally, the gapmer contains a centre region containing non-modified nucleotide residues and two wing regions (5′ and 3′ wing) either side of the centre region, which wing regions each contain at least one modified nucleotide residue. In an embodiment, the polymerase is used to produce the centre region of the gapmer. In one example, the 5′-segment can be used as the primer for the polymerase to produce the centre region of the gapmer, and the 3′-segment can be used as a stopper for the polymerase. A ligase can then be used to fuse the centre segment to the 3′-segment to create the gapmer product. In an embodiment, the polymerase is used to produce part of the centre region of the gapmer. In one embodiment, a 5′-segment can be used as the primer for the polymerase to produce a segment of the gapmer, and the 3′-segment can be used as a stopper sequence for the polymerase. The 5′-segment may comprise or consist of the wing region. The 5′-segment may include part of the wing region. The 3′-segment may comprise or consist of the wing region. The 3′-segment may include part of the wing region. In this way, the complexity of the process is significantly reduced by reducing the number of rounds of production. In an embodiment, the centre region comprises or consists of deoxynucleotides joined by phosphorothioate linkages, i.e. there are no sugar modifications in the centre region, but the backbone is a fully phosphorothioate backbone, and the polymerase is used to produce the centre region of the gapmer. In an embodiment, the 5′-segment is the primer and the 3′-segment is the stopper and each segment comprises or consists of 2′-MOE sugar modified nucleotides joined by phosphorothioate linkages. In an embodiment, the centre region of the gapmer comprises or consists of deoxynucleotides joined by phosphorothioate linkages, the polymerase is used to produce the centre region of the gapmer; and the 5′-segment is the primer and the 3′-segment is the stopper, with each segment comprising or consisting of 2′-MOE sugar modified nucleotides joined by phosphorothioate linkages. In an embodiment, the gapmer has a fully phosphorothioate backbone, a 5′ wing that is fully 2′MOE sugar modified, a 3′ wing that is fully 2′MOE sugar modified, a centre region that is fully deoxyribose, and said gapmer is produced using a 5′-segment that corresponds to the 5′ wing as a primer, a 3′-segment that corresponds to the 3′ wing as a stopper, and a polymerase and a pool of deoxynucleoside alpha-thiotriphosphates to produce the centre region. In an embodiment, the gapmer is a 5-10-5 gapmer.

In traditional polynucleotide manufacturing methods, e.g. solid-phase synthesis, a single nucleotide is added to a single-stranded oligonucleotide, in a template independent manner, allowing synthesis of an oligonucleotide with a defined sequence. Such an approach could be used to produce the full oligonucleotide product by iteratively adding single bases. However, unless each synthetic cycle runs with 100% yield, sequence deletion errors will be incorporated into the final product. For example, if an oligonucleotide is extended by one nucleotide with 99% yield in a synthetic cycle, the remaining 1% will be available to react in subsequent synthetic cycles but the product formed will be one nucleotide shorter than the desired product. As the number of cycles increases then the error rate is compounded so, in this example, a 99% cycle yield would result in the formation of 20% of single base shortened sequences for the production of a 20mer.

Attempts to overcome this using only short sequences, typically 5 to 8 nucleotides long, synthesised by the addition of single nucleotides using a ligase or transferase that is capable of adding a single nucleotide to a single-stranded oligonucleotide have yielded short sequences that have a higher purity than long sequences as they are exposed to fewer cycles of error accumulation. These short sequences are assembled on a complementary DNA template and then joined together. The use of a complementary template in conjunction with a ligase ensures that only short oligonucleotides that have both the correct length and the correct sequence are assembled. However, such methods still require multiple rounds of synthesis to add each nucleotide to another nucleotide in order to create each short sequence, which is then combined. This is time consuming and complex.

Accordingly, the processes of the disclosure result in more efficient processes for producing single-stranded oligonucleotide and polynucleotide products with a high overall yield and high overall sequence fidelity.

According to the present disclosure, by using a polymerase in combination with a ligase, single-stranded oligonucleotide and polynucleotide products can be produced without repeated rounds of chemical synthesis in an efficient process.

The sequential accumulation of errors is also avoided. Firstly, by using at least two segments of the product sequence, and using the polymerase to fill in gaps, this allows multiple shorter sequences to make up the final product, which gives fewer rounds of synthesis, e.g. by solid phase synthesis, in which errors can be introduced. Polymerases also have a high sequence fidelity, reducing the chance for error incorporation. This further reduces limitations on scale-up of the process for commercial oligonucleotide and polynucleotide production.

Secondly, assembly of the final product on a complementary polynucleotide template with subsequent ligation ensures that the segments are assembled in the correct order and chirality, with the correct length and sequence required for the final product. This enables a highly accurate, personalised final product to be created.

In an exemplary embodiment, the process is fully-enzymatic, which allows for reduced rounds of synthesis and increased efficiency. In fact, the polymerase and ligase steps can occur concurrently, such that the final product can be produced in one round of the process. Where the polymerase and ligase steps occur concurrently this may be known as “one-pot”.

In another exemplary embodiment, the method is further simplified and results in further efficiencies by using (in combination with a template polynucleotide or oligonucleotide as described herein) a short primer sequence for the polymerase, which is complementary to the 3′ end of the template. The polymerase then extends the primer sequence along the template. A stopper complementary to the 5′ end of the template can be used to stop the polymerase. A stop sequence can be dispensed with when the correct amount of nucleoside triphosphates are added to the reaction. This reduces the number of rounds of nucleotide addition required and the number of ligations required, resulting in a more efficient process with a high overall yield and high overall sequence fidelity.

In embodiments, the polymerase is modified to remove both the 3′ to 5′ exonuclease activity and the 5′ to 3′ exonuclease activity, as described herein, to reduce or prevent destruction of the segment polynucleotide or oligonucleotide sequences.

In an embodiment, the polynucleotide product is a mRNA and the coding region comprises unmodified nucleotides, except for the replacement of uridine residues with pseudouridine or N1-methyl-pseudouridine. In an embodiment, the polymerase is used to produce part or all of the coding region of the mRNA. In an embodiment, the 5′-UTR, the 3′-UTR or both the 5′-UTR and the 3′-UTR comprise one or more modified nucleotides. In an embodiment, phosphorothioate linkages are included in the 5′-UTR at either cytidine or both cytidine and uridine. In an embodiment, the poly-A tail comprises one or more modifications that are not susceptible to 3′-5′ exonucleases. In an embodiment, the poly-A tail comprises one or more phosphorothioate linkages. In an embodiment, the poly-A tail comprises phosphorothioate linkages at its 3′-end. In an embodiment, the poly-A tail comprises as least 6 phosphorothioate linkages at its 3′ end.

A polynucleotide or oligonucleotide product in accordance with the present disclosure may have at least one modified sugar moiety, modification of the nucleobase, and/or modification of the backbone, as described herein.

In embodiments, one or more segment polynucleotides or oligonucleotides can have at least one modified nucleotide residue. In embodiments, all segment polynucleotides or oligonucleotides have at least one modified nucleotide residue. In other embodiments, one or more segment polynucleotides or oligonucleotides can have no modified nucleotide residues. In embodiments, the polymerase can incorporate at least one modified nucleotide residue into the extended sequence. In other embodiments, the polymerase can incorporate non-modified nucleotide residues into the extended sequence.

In some embodiments, one or more segment polynucleotides or oligonucleotides can have at least one modified nucleotide residue and the polymerase can incorporate at least one modified nucleotide residue into the extended sequence. In some embodiments, one or more segment polynucleotides or oligonucleotides can have at least one modified nucleotide residue and the polymerase can incorporate non-modified nucleotide residues into the extended sequence. In some embodiments, one or more segment polynucleotides or oligonucleotides can have no modified nucleotide residues and the polymerase can incorporate at least one modified nucleotide residue into the extended sequence. In some embodiments, one or more segment polynucleotides or oligonucleotides can have no modified nucleotide residues and the polymerase can incorporate non-modified nucleotide residues into the extended sequence.

In embodiments, the product can be a gapmer. In embodiments, the wing region (optionally 5′- and/or 3′-segment oligonucleotides) can comprise backbone and sugar modifications and the central region can comprise backbone modifications, but no sugar modifications. In embodiments, the wing region can comprise at least one sugar modification or can consist entirely of modified sugars. In embodiments, the 5′ and 3′ wings of the gapmer comprise or consist of 2′-MOE modified nucleotides. In embodiments, the centre region of the gapmer comprises or consists of nucleotides containing hydrogen at the 2′ position of the sugar moiety, i.e. is DNA-like. In embodiments, the 5′ and 3′ wings of the gapmer consist of 2′MOE modified nucleotides and the centre region of the gapmer consists of nucleotides containing hydrogen at the 2′ position of the sugar moiety (i.e. deoxynucleotides). In embodiments, the 5′ and 3′ wings of the gapmer consist of 2′MOE modified nucleotides and the centre region of the gapmer consists of nucleotides containing hydrogen at the 2′ position of the sugar moiety (i.e. deoxynucleotides) and the linkages between all of the nucleotides are phosphorothioate linkages. In an embodiment, the gapmer is selected from the group consisting of: baliforsen, bepirovirsen, custirsen, daplusiran, donidalorsen, eplontersen, frenlosirsen, inotersen, lademirsen, mipomersen, olezarsen, tominersen, ulefnersen, volanesorsen, and zilganersen. In an embodiment the gapmer is a 5-10-5 gapmer. In an embodiment the gapmer is a 6-8-6 gapmer. In an embodiment the gapmer is a 4-12-4 gapmer. In an embodiment the gapmer is a 7-6-7 gapmer.

Provided are processes wherein the resulting product is greater than 90% pure. In embodiments, the product can be greater than 95% pure. In embodiments, the product can be greater than 96% pure. In embodiments, the product can be greater than 97% pure. In embodiments, the product can be greater than 98% pure. In embodiments, the product can be greater than 99% pure. Purity of a polynucleotide or oligonucleotide may be determined using any suitable method, e.g. high-performance liquid chromatography (HPLC) or mass spectrometry (MS), in particular, liquid chromatography-MS (LC-MS), HPLC-MS or capillary electrophoresis mass spectrometry (CEMS).

In an embodiment, the single-stranded polynucleotide or oligonucleotide produced is selected from the group consisting of: alicaforsen, Apc001PE, AS1411, baliforsen, bepirovirsen, BIIB080, BIIB094, BIIB101, BIIB105, BIIB115, BIIB121, BIIB132, casimersen, cimdelirsen, CpG1018, CpG7909, custirsen, daplusiran, donidalorsen, drisapersen, eplontersen, eteplirsen, fesomersen, fomiversen, frenlosirsen, golodirsen, imetelstat, inotersen, ION224, ION260, ION306, ION363, ION455, ION464, ION532, ION541, ION582, ION839, ION859, ION904, IONIS-AGT-L_{Rx}, IONIS-FB-L_{Rx}, IONIS-MAPT_{Rx}, lademirsen, lexanersen, mipomersen, mongersen, nusinersen, NOX-E36, olezarsen, pegaptanib, pelacarsen, prexigebersen, renadirsen, RG6048, rugonersen, sapablursen, sepofarsen, suvodirsen, tofersen sodium, tominersen, ulefnersen, ultevursen, vesleteplirsen, viltolarsen, volanesorsen, WVE-003, WVE-004, WVE-006, WVE-N531, and zilganersen.

In embodiments, the polynucleotide or oligonucleotide produced is an antisense polynucleotide or oligonucleotide. In an embodiment, the antisense oligonucleotide is selected from the group consisting of: alicaforsen, baliforsen, bepirovirsen, BIIB080, BIIB094, BIIB101, BIIB105, BIIB115, BIIB121, BIIB132, cimdelirsen, casimersen, custirsen, daplusiran, donidalorsen, drisapersen, eplontersen, eteplirsen, fesomersen, fomiversen, frenlosirsen, golodirsen, imetelstat, inotersen, ION224, ION260, ION306, ION363, ION455, ION464, ION532, ION541, ION582, ION839, ION859, ION904, IONIS-AGT-L_{Rx}, IONIS-FB-L_{Rx}, IONIS-MAPT_{Rx}, lademirsen, lexanersen, mipomersen, mongersen, nusinersen, olezarsen, pelacarsen, prexigebersen, renadirsen, RG6048, rugonersen, sapablursen, sepofarsen, suvodirsen, tofersen sodium, tominersen, ulefnersen, ultevursen, vesleteplirsen, viltolarsen, volanesorsen, WVE-003, WVE-004, WVE-N531, and zilganersen.

In embodiments, the polynucleotide or oligonucleotide produced is an aptamer. In an embodiment, the aptamer is pegaptanib, Apc001PE, AS1411 or NOX-E36.

In embodiments, the polynucleotide or oligonucleotide produced is a mRNA, such as Cas-9. In one embodiment, the polynucleotide or oligonucleotide produced is an mRNA vaccine. In an embodiment, the mRNA encodes one or more immunogens. In a further embodiment, these immunogens may be selected from a respiratory syncytial virus (RSV) immunogen, an Epstein-Barr virus glycoprotein immunogen, a cytomegalovirus glycoprotein immunogen, a coronavirus spike polypeptide immunogen, an influenza virus immunogen, a Varicella zoster virus glycoprotein immunogen, a human papillomavirus 16 (HPV16) E6 immunogen, a HPV 16 E7 immunogen, or a flavivirus immunogen. In a further embodiment, those immunogens may be selected from a coronavirus spike protein, an influenza antigen, and RSV antigen such as protein f or protein g.

In an embodiment the mRNA is selected from the group consisting of AZD8601, BNT111, BNT112, BNT113, BNT115, BNT116, BNT122, BNT131, BNT141, BNT142, BNT151, BNT152, BNT153, BNT161, BNT162b2, BNT163 BNT164, BNT165, LUNAR-CF, LUNAR-COV19, LUNAR-FLU, LUNAR-GSDIII, LUNAR-OTC, MEDI1191, mRNA-1273, mRNA-0184, mRNA-1010, mRNA-1020, mRNA-1030, mRNA-1011, mRNA-1012, mRNA-1045, mRNA-1073, mRNA-1189, mRNA-1195, mRNA-1215, mRNA-1230, mRNA-1273, mRNA-1273.211, mRNA-1273.213, mRNA-1273.214, mRNA-1273.222, mRNA-1273.351, mRNA-1273.529, mRNA-1273.617, mRNA-1283, mRNA-1287, mRNA-1345, mRNA-1365, mRNA-1468, mRNA-1574, mRNA-1608, mRNA-1644, mRNA-1647, mRNA-1653, mRNA-1893, mRNA2752, mRNA-3139, mRNA-3283, mRNA-3351, mRNA-3705, mRNA-3745, mRNA-3927, mRNA-4157, mRNA-4359, mRNA-5671, mRNA-6981, and VX-522. In a further embodiment, the mRNA is selected from the group consisting of mRNA-1045, mRNA-1230, mRNA-1345, mRNA-1365, EBV mRNA-1189, mRNA-1195, mRNA-1647, BNT162b2, LUNAR-COV19, mRNA-1073, mRNA-1273.213, mRNA-1273.214, mRNA-1273.222, mRNA-1273.351, mRNA-1273.529, mRNA-1273.617, mRNA-1283, mRNA-1287, mRNA-1345, BNT161, mRNA-1468, BNT113, and mRNA-1893. In a further embodiment, the mRNA is selected from the group consisting of mRNA-1045, mRNA-1230, mRNA-1345, mRNA-1365, BNT162b2, LUNAR-COV19, mRNA-1073, mRNA-1273.213, mRNA-1273.214, mRNA-1273.222, mRNA-1273.351, mRNA-1273.529, mRNA-1273.617, mRNA-1283, mRNA-1287, mRNA-1345, BNT161, and LUNAR-FLU.

In an embodiment, the polynucleotide or oligonucleotide product is an adjuvant. In an embodiment, the adjuvant is a CpG oligonucleotide. In an embodiment, the adjuvant is CpG1018 or CpG7909.

In embodiments, the product is a therapeutic polynucleotide or oligonucleotide.

In embodiments, the process can produce double-stranded polynucleotides or oligonucleotides, wherein two complementary single-stranded polynucleotides or oligonucleotides are produced by the method as described herein and then mixed under conditions to allow annealing, such conditions being readily apparent to the skilled person. In an embodiment, the product is a siRNA. In an embodiment, the siRNA is selected from the group consisting of: ALN-AAT02, ALN-APP, ALN-TTRsc04, ALN-HBV02, ALN-HSD, ALN-KHK, ALN-PNP, ARO-AAT, ARO-ANG3, AOC 1001, AOC 1020, AOC 1044, ARO-APOC3, ARO-C3, ARO-COV, ARO-DUX4, ARO-ENaC2, ARO-MUC5AC, ARO-MMP7, ARO-PNPLA3, ARO-RAGE, belcesiran, cemdisiran, cosdosiran, daplusiran, DCR-AUD, DCR-CM3, DCR-CM4, DCR-COMP1, DCR-COMP2, DCR-LIV2, DCR-LLY11, DCR-LLY12, DCR-NOVO1, DCR-NOVO2, elebsiran, fazirsiran, fitusiran, givosiran, HZN-457, inclisiran, JNJ-3989, lumasiran, LY3561774, LY3819469, nedosiran, olpasiran, patisiran, revusiran, RG6346, RIM730, SLN124, SLN501, SLN-HAN-1, SLN-MNK-2, SLN-MNK-3, SLN-AZ-1, SLN-AZ-2, STP122G, STP125G, STP135G, STP136G, STP144G, STP145G, STP146G, STP152G, STP155G, STP237G, STP247G, STP251G, STP355, STP369, STP702, STP779, STP705, STP707, teprasiran, tivanisiran, vutrisiran, zerlasiran, zilebesiran, and zifcasiran. In an embodiment, the product is a miRNA or miRNA mimic. In an embodiment, the miRNA mimic is remlarsen.

The invention herein disclosed utilises the properties of oligonucleotide binding to provide an improved process for their production. By providing a template oligonucleotide with 100% complementarity to the target sequence, and controlling the reaction conditions so that the product can be released and separated under specific conditions, a product with a high degree of purity can be obtained.

Denaturing the Product (or Impurity):Template Duplex and Separating the Product (or Impurity) from the Template

Releasing the product (or any impurities) from the template requires the Watson-Crick base pairing between the template polynucleotide or oligonucleotide strand and the product (or impurity) to be broken (i.e. denaturing the duplex). The product (or impurity) can then be separated from the template, which can occur as two separate steps, or as one combined step.

Releasing and separating the product (or impurity) can occur as one step, if the process is carried out in a column reactor. Running in a buffer that alters the pH or salt concentration, or contains a chemical agent that disrupts the base pairing (such as formamide or urea) will cause denaturation of the polynucleotide or oligonucleotide strands, and the product (or impurity) will be eluted in the buffer.

When the process is carried out in other reaction vessels, the release and separation of the product (or impurity) can occur as a two-step process. First, the Watson-Crick base pairs are disrupted to denature the strands, and then the product (or impurity) is separated from the template, e.g. removed from the reaction vessel. When releasing and separating the product is carried out as a two-step process, the breaking of the Watson-Crick base pairs can be achieved by altering the buffer conditions (pH, salt) or by introducing a chemical disrupting agent (formamide, urea). Alternatively, raising the temperature will also cause the dissociation of the two strands, i.e. denaturation. The product (or impurities) can then be separated (and also removed from the reaction vessel, if desired) via methods including molecular weight-based separation, charge-based separation, hydrophobicity-based separation, specific sequence-based separation or a combination of these methods.

When the process is carried out in a continuous or semi-continuous flow reactor, the release and separation of the product (or impurity) can be in either one step or two steps. For example, releasing and separating the product (or impurity) in one step could be affected by increasing the temperature to cause dissociation of the two strands, and separating the released strands on the basis of molecular weight in the same part of the reactor that is used to elevate the temperature. Releasing and separating the product (or impurity) in two steps could be affected by increasing the temperature to cause dissociation of the two strands in one part of the reactor and separating the released strands on the basis of molecular weight in a different part of the reactor.

Specifically Releasing and Separating Impurities from the Template, but Retaining the Product on the Template

Impurities arise when an incorrect nucleotide is incorporated into the oligonucleotide strand during chain extension, or when the chain extension reaction terminates early. Impurities also arise when the reaction includes the step of ligating segment polynucleotides or oligonucleotides and one or more of the ligation steps fail to happen.

The properties of Watson-Crick base pairing can be exploited to specifically release any impurities bound to the template prior to the release of the product. Each double-stranded polynucleotide or oligonucleotide will dissociate under specific conditions, and those conditions are different for sequences which do not have 100% complementarity when compared to sequences with 100% complementarity. Determining such conditions is within the remit of a skilled person.

A common way of denaturing polynucleotides or oligonucleotides is by raising the temperature. The temperature at which half of the base pairs are dissociated, i.e. when 50% of the duplex is in the single-stranded state, is called the melting temperature, Tₘ. The most reliable and accurate means of determining the melting temperature is empirically. However, this is cumbersome and not usually necessary. Several formulas can be used to calculate Tₘ values (Nucleic Acids Research 1987, 15 (13): 5069-5083; PNAS 1986, 83 (11): 3746-3750; Biopolymers 1997, 44 (3): 217-239) and numerous melting temperature calculators can be found on-line, hosted by reagent suppliers and universities. It is known that for a given polynucleotide or oligonucleotide sequence, a variant with all phosphorothioate linkages will melt at a lower temperature than a variant with all phosphodiester linkages. Increasing the number of phosphorothioate linkages in an polynucleotide or oligonucleotide tends to lower the Tₘ of the polynucleotide or oligonucleotide for its intended target.

To specifically separate the impurities from a reaction mixture, first the melting temperature of the product:template duplex is calculated. Then the reaction vessel is heated to a first temperature, e.g. a temperature below the melting temperature of the product:template duplex, for example 1, 2, 3, 4, 5, 6, 7, 8, 9 or 10 degrees centigrade below the melting temperature. This heating step causes the denaturing of polynucleotides or oligonucleotides which are not the product, i.e. are not 100% complimentary to the template, from the template. These denatured polynucleotides or oligonucleotides can then be removed from the reaction vessel using one of the methods disclosed above, e.g. molecular weight-based separation, charge-based separation, hydrophobicity-based separation, specific sequence-based separation or a combination of these methods. Then, the reaction vessel will be raised to a second, higher, temperature, e.g. above the calculated melting temperature, for example 1, 2, 3, 4, 5, 6, 7, 8, 9 or 10 degrees centigrade above the melting temperature, to cause the denaturing of the product from the template. The product can then be separated (and removed from the reaction vessel) using one of the methods disclosed above, e.g. molecular weight-based separation, charge-based separation, hydrophobicity-based separation, specific sequence-based separation or a combination of these methods.

A similar process can be used when the disrupting agent is an agent which causes a change in pH or salt concentration or is a chemical disrupting agent. The disrupting agent is increased in concentration until just below the concentration at which the product would dissociate, to cause the denaturing of polynucleotides or oligonucleotides which are not the product from the template. These impurities can then be removed from the reaction vessel using one of the methods disclosed above. The disrupting agent is then increased in concentration to above the concentration at which the product dissociates from the template. The product can then be removed from the reaction vessel using one of the methods disclosed above.

The product obtained from a process such as disclosed above has a high degree of purity without the need for further purification steps. For example, the product obtained is greater than 95% pure.

### Properties of the Template

In some embodiments, the template can have a property which allows it to be retained in the reaction vessel when the product is removed, to prevent it from becoming an impurity in the product. In one embodiment of the invention, this retention is achieved by coupling the template to a supporting material. This coupling results in a template-support complex which has a high molecular weight, and can therefore be retained in the reaction vessel when impurities and product are removed, for example by filtration. The template can be coupled to a solid support material such as polymeric beads, fibrous supports, membranes, streptavidin coated beads and cellulose. The template can also be coupled to a soluble support material such as polyethylene glycol, a soluble organic polymer, DNA, a protein, a dendrimer, a polysaccharide, an oligosaccharide and a carbohydrate.

Each support material can have multiple points where a template can be attached, and each attachment point can have multiple templates attached.

The template may have a high molecular weight itself, without being attached to a support material, for example, it may be a molecule with multiple copies of the template, e.g. separated by a linker, in the manner shown in FIG. 2.

The ability to retain the template in the reaction vessel also allows the template to be recycled for future reactions, either by being recovered or by use in a continuous or semi-continuous flow process. This is useful, for example, in oligonucleotide production, such as gapmer production, and in therapeutic mRNA production.

The properties of the template can allow separation of the template and product, or separation of the template bound product and impurities. Molecular weight-based separation, charge-based separation, hydrophobicity-based separation, specific sequence-based separation or a combination of these methods can be used.

In the case where the template is attached to a solid support, separation of the template from the product, or separation of impurities from the product bound to the template, is achieved by washing the solid support under appropriate conditions as would be readily apparent to a person skilled in the art. In cases where the template is coupled to a soluble support or is itself composed of repeating template sequences, separation of template from product or separation of template bound product from impurities can be achieved by means of a molecular weight-based separation, for example by using techniques such as ultra-filtration or nano-filtration where the filter material is chosen so that the larger molecule is retained by the filter and the smaller molecule passes through. In cases where a single separation step of impurity from template product complex, or separation of product from template, is not efficient enough, multiple sequential filtration steps can be employed to increase separation efficiency and so generate a product that meets the desired purity.

It is desirable to provide a process for separation of such polynucleotides or oligonucleotides which is efficient and applicable on an industrial production scale. “Therapeutic oligonucleotides: The state of the art in purification technologies” Sanghvi et. al. Current Opinion in Drug Discovery (2004) Vol. 7 No. 8 reviews processes used for oligonucleotide purification.

WO 01/55160 A1 discloses purification of oligonucleotides by forming imine linkages with contaminants and then removing the imine-linked impurities with chromatography or other techniques. “Size Fractionation of DNA Fragments Ranging from 20 to 30000 Base Pairs by Liquid/Liquid chromatography” Muller et al. Eur. J. Biochem (1982) 128-238 discloses use of a solid column of microcrystalline cellulose on which has been deposited a PEG/dextran phase for separation of nucleotide sequences. “Separation and identification of oligonucleotides by hydrophilic interaction chromatography.” Easter et. al. The Analyst (2010); 135(10) discloses separation of oligonucleotides using a variant of HPLC employing a solid silica support phase. “Fractionation of oligonucleotides of yeast soluble ribonucleic acids by countercurrent distribution” Doctor et al. Biochemistry (1965) 4(1) 49-54 discloses use of a dry solid column packed with dry DEAE-cellulose. “Oligonucleotide composition of a yeast lysine transfer ribonucleic acid” Madison et al; Biochemistry, 1974, 13(3) discloses use of solid phase chromatography for separation of nucleotide sequences.

Liquid-liquid chromatography is a known separation method. “Countercurrent Chromatography The Support-Free Liquid Stationary Phase” Billardello, B.; Berthod, A; Wilson & Wilson's Comprehensive Analytical Chemistry 38; Berthod, A., Ed.; Elsevier Science B.V.: Amsterdam (2002) pp 177-200 provides a useful general description of liquid-liquid chromatography. Various liquid-liquid chromatography techniques are known. One such technique is liquid-liquid counter current chromatography (termed herein “CCC”). Another known technique is centrifugal partition chromatography (termed herein “CPC”).

The above disclosed methods and those methods set out in WO 2013/030263 may be used to separate a product polynucleotide or oligonucleotide, e.g. from the template and/or an impurity.

### Polymerases

In embodiments, the polymerase used can be a DNA polymerase, an RNA polymerase or a combination thereof. The polymerase can be a mutant polymerase. The polymerase can be an engineered polymerase. The polymerase can catalyse the joining of a deoxyribonucleotide to a deoxyribonucleotide, a deoxyribonucleotide to a ribonucleotide, a ribonucleotide to a deoxyribonucleotide and/or a ribonucleotide to a ribonucleotide. The polymerase can lack strand-displacement activity. The polymerase can lack 5′ to 3′ exonuclease activity. Lack of 5′ to 3′ exonuclease activity can prevent destruction of the stopper. The polymerase can have 3′ to 5′ exonuclease activity. 3′ to 5′ exonuclease activity typically enables a polymerase to remove misincorporated nucleotides and thereby ensures high-fidelity synthesis. 3′ to 5′ exonuclease activity may be known as proof reading ability. The polymerase may lack 3′ to 5′ exonuclease activity. Different polymerase properties may be combined as necessary, e.g. the polymerase may lack strand-displacement activity and/or lack 5′ to 3′ exonuclease activity and/or have 3′ to 5′ exonuclease activity. For example, the polymerase may lack strand-displacement activity and/or lack 5′ to 3′ exonuclease activity and/or lack 3′ to 5′ exonuclease activity. The polymerase can include DNA and/or RNA polymerases. The polymerase can be a DNA-dependent DNA polymerase (i.e. a polymerase that synthesises DNA using a DNA template). The polymerase can be a DNA-dependent RNA polymerase (i.e. a polymerase that synthesises RNA using a DNA template). The DNA polymerase can be capable of joining deoxyribonucleotides and/or ribonucleotides. The RNA polymerase can be capable of joining ribonucleotides and/or deoxyribonucleotides. The polymerase can be a primer extending polymerase. The DNA polymerase can be a primer extending polymerase. The RNA polymerase can be a primer extending polymerase. The DNA and/or RNA polymerase can be a wild-type polymerase. The DNA and/or RNA polymerase can be a mutant DNA and/or RNA polymerase. The DNA and/or RNA polymerase can be an engineered DNA and/or RNA polymerase. The polymerase can be capable of joining modified nucleotides. The DNA and/or RNA polymerase can be capable of joining modified nucleotides. The polymerase can fill in at least one sequence gap along the template strand. The polymerase can extend at least one segment polynucleotide using a pool of nucleoside triphosphates. DNA polymerases typically require a primer and a template. Exemplary polymerases include wild-type Escherichia phage T7 polymerase, wild-type Sulfolobus solfataricus polymerase, mutant Thermococcus sp. (strain 9oN-7) polymerase, wild-type Enterobacteria phage T4 polymerase, wild-type Thermus aquaticus polymerase, and wild-type Thermococcus kodakarens polymerase.

Within the scope of the disclosure is a polymerase capable of joining an unmodified nucleotide to another unmodified nucleotide, a polymerase capable of joining an unmodified nucleotide to a modified nucleotide (i.e. a modified 5′ nucleotide to an unmodified 3′ nucleotide, and/or an unmodified 5′ nucleotide to a modified 3′ nucleotide), as well as a polymerase capable of joining a modified nucleotide to another modified nucleotide. The polymerase is capable of joining an unmodified nucleotide to another unmodified nucleotide. Each unmodified nucleotide can subsequently be modified. Examples of modifications of nucleotides are disclosed herein, and include modifications selected from the group comprising a modified sugar moiety, modification of the nucleobase, modification of the backbone, substitution of one or more uracil residues and combinations thereof.

Additionally, exemplary engineered DNA and RNA polymerases that can incorporate modified nucleotides include those disclosed in “Engineering and application of polymerases for synthetic genetics”, Houlihan et al., Current Opinion in Biotechnology 2017, 48; 168-179.

In embodiments, the DNA and/or RNA polymerases can be engineered to accept 2′ sugar modifications, including polymerases with mutations in the polymerase thumb subdomain of Thermococcus gogonarius replicative DNA polymerase, optionally comprising mutations at E664K and Y409G. Such polymerases provide for the inclusion of, for example, pseudouridine, 5-methyl-C, 2′-fluoro, or 2-azdio-modified NTPs primed from DNA, RNA, Locked nucleic acid, or 2′-OMe RNA modified nucleotides, or combinations thereof.

In embodiments, RNA polymerases engineered to accept 2′ sugar modifications include T7 RNA polymerases. T7 RNA polymerases comprising a mutation at Y639F can, for example, provide for the inclusion of, for example, 2′fluoro pyrimidines and 2′ amino pyrimidines.

In embodiments, variants of the Stoffel fragment of Taq polymerase (SM19) that have been engineered to accept 2′ sugar modifications are used. For example, introduction of a negatively charged amino acid at 614 and mutation of E615G, provide for the inclusion of 2′ sugar modifications. SM19 can be further evolved to polymerase SFM4-3 and SFM4-9. For example, SFM4-3 can transcribe fully modified 2′-OMe 60 nucleotide sequences.

In embodiments, thermophilic RNA polymerase from the marine cyanophage Syn5 that has been engineered to accept 2′ sugar modifications are used.

In embodiments, Tgo polymerases comprising mutations at Y409G, I521L, F545L, and E664K are used, which can synthesise DNA and RNA with regioisomeric 2′-5′ linkages by incorporation of 3′-deoxy- or 3′-OMe nucleotides.

Compartmentalised self-replication methods are useful in evolving polymerases to incorporate modified nucleotides. “Directed evolution of polymerase function by compartmentalized self-replication”, Ghadessy et al., Proc. Natl. Acad. Sci. U.S.A 2001, 98:4552-4557, describe exemplary compartmentalised self-replication methods.

### Ligases

In embodiments, the ligase can be an ATP dependent ligase. ATP dependent ligases range in size from 30 to >100 kDa. In embodiments, the ligase can be an NAD dependent ligase. NAD dependent enzymes are highly homologous and are monomeric proteins of 60 to 90 kDa, optionally 70-80 kDa. In embodiments, the ligase can be a thermostable ligase. A thermostable ligase may be derived from a thermophilic bacterium.

In embodiments, the ligase can be a template-dependent ligase. In embodiments, ligation occurs on the template. In embodiments, ligating segment polynucleotides and/or extended segment polynucleotides occurs on the template using a ligase to form the single-stranded polynucleotide product. In embodiments, the ligase can be a duplex-acting ligase. The duplex can be a duplex DNA. The duplex can be a RNA:DNA hybrid duplex. The duplex can be a duplex RNA. The ligase can be a DNA ligase. The ligase can be an RNA ligase.

In embodiments, the ligase can catalyse the joining of two segment polynucleotides and/or extended segment polynucleotides. In embodiments, the ligase can catalyse the joining of two segment oligonucleotides. The ligase can catalyse the joining of one segment polynucleotide or extended segment polynucleotide comprising naturally occurring nucleotides and one segment polynucleotide extended segment polynucleotide comprising naturally occurring nucleotides. The ligase can catalyse the joining of one segment polynucleotide or extended segment polynucleotide comprising at least one modified nucleotide and one segment polynucleotide or extended segment polynucleotide comprising naturally occurring nucleotides. The one segment polynucleotide or extended segment polynucleotide comprising at least one modified nucleotide may be positioned at the 3′ end of the join. The one segment polynucleotide or extended segment polynucleotide comprising naturally occurring nucleotides may be positioned at the 5′ end of the join. The modified nucleotide may or may not be positioned at the join, i.e. one or both or none of the junction nucleotides are modified nucleotides. The ligase can catalyse the joining of one segment polynucleotide or extended segment polynucleotide comprising at least one modified nucleotide and one segment polynucleotide or extended segment polynucleotide comprising at least one modified nucleotide. The skilled person would appreciate that the positioning of the at least one modified nucleotide within the segment polynucleotide or extended segment polynucleotide can be adapted to increase ligation efficiency.

The ligase can be wild-type ligase. The ligase can be mutant ligase. The ligase can be an engineered ligase. The DNA and/or RNA ligase can be a wild-type DNA and/or RNA ligase. The DNA and/or RNA ligase can be a mutant DNA and/or RNA ligase. The DNA and/or RNA ligase can be an engineered DNA and/or RNA ligase. The skilled person would appreciate that an appropriate ligase can be selected based on the segment polynucleotides and/or extended segment polynucleotides that require ligation and/or the number and/or type and/or position of modifications in said segment polynucleotides and/or extended segment polynucleotides.

Exemplary ligases include wild-type Enterobacteria phage T3 ligase and wild-type bacteriophage T4 DNA ligase.

In embodiments, the ligase can be immobilised, e.g. on a bead.

### Pool of Polynucleotides or Polynucleotides

The polynucleotides or oligonucleotides used to create the “pool of polynucleotides” of the processes of the disclosure may comprise at least two segments of the product sequence. The polynucleotides or oligonucleotides used to create the pool of the processes of the disclosure may comprise at least two different segments of the product sequence. The at least two segments of the product sequence may differ in sequence. The at least two segments each correspond to different regions of the product sequence. The pool of polynucleotides or oligonucleotides may comprise at least one segment polynucleotide or oligonucleotide comprising at least one modified nucleotide residue. The pool of polynucleotides or oligonucleotides may comprise at least two segment polynucleotides or oligonucleotides, wherein each segment polynucleotide or oligonucleotide comprises at least one modified nucleotide residue.

The pool is thus a non-homogenous set of polynucleotides or oligonucleotides. The at least two segment polynucleotides or oligonucleotides vary in sequence, may be shorter than the target sequence, and may not have the same sequence as the target sequence.

The at least two segment polynucleotides or oligonucleotides can be produced by enzymatic synthesis, chemical synthesis, optionally solid-supported synthesis or solution-phase synthesis or a combination thereof. Enzymatic synthesis can be performed using a single-stranded ligase, a transferase, a polymerase or a combination thereof.

In other examples, one or more or all of the segment polynucleotides or oligonucleotides can be produced using chemical synthesis. In other examples, one or more or all of the segment polynucleotides or oligonucleotides can be produced using chemical synthesis in combination with enzymatic synthesis, for example the use of a polymerase, single-stranded ligase or transferase, or a combination thereof. In other examples, one or more or all of the segment polynucleotides or oligonucleotides can be produced using chemical synthesis, a single-stranded ligase and/or a transferase in combination with the use of a polymerase to produce one or more of the segment oligonucleotides. In other examples, one or more or all of the segment polynucleotides or oligonucleotides can be produced using a single-stranded ligase, a transferase, a polymerase of a combination thereof in a fully enzymatic method of synthesis. In a simplified method, a single-stranded ligase and a polymerase can be used in a fully enzymatic method for the production of the pool of the two or more or all of the segment polynucleotides or oligonucleotides.

In an example of oligonucleotide production, the pool of oligonucleotides can comprise a 5′ primer segment oligonucleotide that has been produced using chemical synthesis and/or enzymatic synthesis (e.g. using a single-stranded ligase, transferase and/or polymerase), and comprises at least one modified nucleotide residue; and a different 3′-segment oligonucleotide that has been produced using chemical synthesis and/or enzymatic synthesis (e.g. using a single-stranded ligase, transferase and/or polymerase), and comprises at least one modified nucleotide residue.

The segment polynucleotide or oligonucleotide may act as a primer. The segment polynucleotide or oligonucleotide may act as a stopper. The segment polynucleotide or oligonucleotide may act as primer and a stopper. The segment polynucleotide or oligonucleotide may act as primer and a stopper where there are sequence gaps on either side of the segment polynucleotide or oligonucleotide.

In one embodiment, in any of the processes of the disclosure, a segment oligonucleotide may be provided which is a 5′ primer segment oligonucleotide comprising at least 3 nucleotides. In one embodiment, a segment oligonucleotide may be provided which is a 5′ primer segment oligonucleotide comprising between 3 to 50 nucleotides. In one embodiment, a segment oligonucleotide may be provided which is a 5′ primer segment oligonucleotide comprising between 3 to 40 nucleotides. In one embodiment, a segment oligonucleotide may be provided which is a 5′ primer segment oligonucleotide comprising between 3 to 30 nucleotides. In one embodiment, a segment oligonucleotide may be provided which is a 5′ primer segment oligonucleotide comprising between 3 to 25 nucleotides. In one embodiment, a segment oligonucleotide may be provided which is a 5′ primer segment oligonucleotide comprising between 3 to 20 nucleotides. In some embodiments, the 5′ primer segment oligonucleotide comprises at least 5 nucleotides. In some embodiments, the 5′ primer segment oligonucleotide comprises 5, 6, 7, 8, 9, 10, 11, 12, 13, 14, 15, 16, 17, 18, 19, 20 nucleotides, wherein optionally one or more of the nucleotides are modified nucleotides. In some embodiments, the modified nucleotide in the 5′ primer segment oligonucleotide is a pseudouridine, N1-methylpseudouridine, 5-Me, 2′-F, 2′OMe or 2′MOE.

In one embodiment, a segment oligonucleotide may be provided which is a 3′ stopper segment oligonucleotide comprising at least 3 nucleotides. In one embodiment, a segment oligonucleotide may be provided which is a 3′ stopper segment oligonucleotide comprising between 3 to 50 nucleotides. In one embodiment, a segment oligonucleotide may be provided which is a 3′ stopper segment oligonucleotide comprising between 3 to 40 nucleotides. In one embodiment, a segment oligonucleotide may be provided which is a 3′ stopper segment oligonucleotide comprising between 3 to 30 nucleotides. In one embodiment, a segment oligonucleotide may be provided which is a 3′ stopper segment oligonucleotide comprising between 5 to 30 nucleotides. In one embodiment, a segment oligonucleotide may be provided which is a 3′ stopper segment oligonucleotide comprising between 3 to 20 nucleotides. In some embodiments, the 3′ stopper segment oligonucleotide comprises at least 5 nucleotides. In some embodiments, the 3′ stopper segment oligonucleotide comprises 5, 6, 7, 8, 9, 10, 11, 12, 13, 14, 15, 16, 17, 18, 19, 20, 21, 22, 23, 24, 25, 26, 27, 28, 29 or 30 nucleotides, wherein optionally one or more of the nucleotides are modified nucleotides. In some embodiments, the modified nucleotide in the 3′ stopper segment oligonucleotide is a pseudouridine, N1-methylpseudouridine, 5-Me, 2′-F, 2′OMe or 2′MOE.

In one embodiment, a segment oligonucleotide may be provided which is a 5′ primer segment oligonucleotide comprising between 3 to 50 nucleotides, together with a segment oligonucleotide which is a 3′ stopper segment oligonucleotide comprising between 3 to 50 nucleotides. In one embodiment, a segment oligonucleotide may be provided which is a 5′ primer segment oligonucleotide comprising between 3 to 25 nucleotides, together with a segment oligonucleotide which is a 3′ stopper segment oligonucleotide comprising between 3 to 30 nucleotides. In one embodiment, a segment oligonucleotide may be provided which is a 5′ primer segment oligonucleotide comprising between 5 to 25 nucleotides, together with a segment oligonucleotide which is a 3′ stopper segment oligonucleotide comprising between 5 to 30 nucleotides. In one embodiment, a segment oligonucleotide may be provided which is a 5′ primer segment oligonucleotide comprising between 5 to 20 nucleotides, together with a segment oligonucleotide which is a 3′ stopper segment oligonucleotide comprising between 5 to 30 nucleotides.

### Producing Segment Polynucleotides or Oligonucleotides Enzymatically

### 1) Polymerase

Polymerases can catalyse the linking of the 3′-hydroxyl group of the end nucleotide of a short oligonucleotide (primer) to the 5′-phosphate of the nucleotide to be added in a template dependent manner. The nucleotide to be added (i.e. the nucleoside triphosphate in the pool of nucleotide trisphosphates) can be non-modified, i.e. naturally occurring, or modified as described herein. A separate template and primer can be used or a self-priming template can be used. The polynucleotide or oligonucleotide can be subsequently modified or further modified.

2) Single-Stranded Ligase, e.g. RNA Ligase

Single-stranded ligases catalyse the ATP driven addition of, for example, 3′,5′ nucleotide bisphosphates, 3′,5′ nucleotide thiophosphates (e.g. 3′,5′ bisthiophosphate or 3′-phosphate-5′-thiophosphate or 3′-thiophosphate-5′-phosphate) or 3′,5′ nucleotide dithiophosphates (e.g. 3′,5′ bisdithiophosphate or 3′-phosphate-5′-dithiophosphate or 3′-dithiophosphate-5′-phosphate) to the 3′-OH of a short oligonucleotide (primer) in a template-independent manner. A skilled person would appreciate that diphosphates (or dithiophosphates) or triphosphates (or other oligophosphates where one or more oxygen atoms has been substituted by sulphur) at the 3′ position of the sugar moiety may also be used, although the additional phosphate (or thiophosphate) moieties are not required. An equivalently modified dinucleotide, trinucleotide or tetranucleotide may be used instead of the aforementioned individual nucleotides. The oligonucleotide primer is usually a minimum of three nucleotides long. The resulting polynucleotide or oligonucleotide of this addition reaction is one nucleotide longer than the starting polynucleotide or oligonucleotide (or two, three or four nucleotides longer than the starting polynucleotide or oligonucleotide if a dinucleotide, trinucleotide or tetranucleotide is used, respectively). The nucleotide introduced can be non-modified, i.e. naturally occurring, or modified as described herein. The new 3′ position is now phosphorylated. In order to add a subsequent nucleotide, the 3′ phosphate of the growing polynucleotide or oligonucleotide is removed to generate a 3′-OH by hydrolysis. This hydrolysis is typically done using a phosphatase enzyme.

For example, a single-stranded ligase can be used in a method of producing a segment polynucleotide or oligonucleotide, wherein the method using the single-stranded ligase comprises 3′-extension for segment synthesis comprising a two-step reaction: addition and deprotection. The exemplary addition step involves ATP dependent ligation of nucleotide-3′,5′-bis(thio)phosphate on to the 3′-OH of a single-stranded nucleic acid primer and then deprotection of the 3′-phosphate on the single-stranded polynucleotide or oligonucleotide by a phosphatase. In another example, a single-stranded ligase can be used in a method of producing a segment polynucleotide or oligonucleotide wherein the method comprises the exemplary 3′-extension (addition and deprotection) to produce a segment sequence followed by chain cleavage using a site-specific nuclease (e.g. endonuclease V—cleaves one base after inosine, i.e. at second phosphodiester bond 3′ to inosine) to release the segment.

### 3) Transferase

Terminal deoxynucleotidyl transferase (TdT) enzymes catalyse the addition of 3′-protected nucleotide triphosphates, e.g. protected by a 3′-0-azidomethyl, 3′-aminoxy or 3-0-allyl group, to the 3′-OH of a short oligonucleotide (primer) in a template-independent manner. This oligonucleotide primer is usually a minimum of three nucleotides long. The nucleotide introduced can be non-modified, i.e. naturally occurring, or modified as described herein.

Suitable methods are set out, for example, in EP2796552, U.S. Pat. No. 8,808,989, WO16128731 A1 and WO16139477 A1.

The primer oligonucleotide used in the above-described methods for producing segment polynucleotides or oligonucleotides can:
(1) be retained as part of the segment polynucleotide or oligonucleotide if desired, or
(2) be cleaved from the product polynucleotide or oligonucleotide to allow separation of the desired product and allow for the possibility of recycling to make further segment polynucleotides or oligonucleotides. Cleavage of the primer from the segment polynucleotide or oligonucleotide can be performed using a sequence specific nuclease and an appropriate design of primer and segment such that the cleavage is both effective and precise.

The present invention is illustrated by the following clauses:

1. A process for producing a single-stranded polynucleotide product having at least one modified nucleotide residue, the process comprising:
a) providing a template polynucleotide comprising a sequence complementary to the single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two segment polynucleotides;
d) providing a pool of nucleotides and a polymerase, and extending at least one segment polynucleotide using the pool of nucleotides and the polymerase, to fill in the at least one sequence gap;
e) ligating segment polynucleotides and/or extended segment polynucleotides using a ligase to form the single-stranded polynucleotide product;
f) optionally, changing the conditions to denature the annealed template and any impurities, and separating the impurities; and
g) changing the conditions to denature the annealed template and the single-stranded polynucleotide product, and separating the single-stranded polynucleotide product.
2. The process according to clause 1, wherein at least one segment polynucleotide comprises at least one modified nucleotide residue.
3. The process according to clause 1 or clause 2, wherein at least one segment polynucleotide comprises a 5′ phosphate, a 5′ phosphorothioate, 5′ phosphorodithioate or 5′ methylphosphonate.
4. The process according to any one of clauses 1 to 3, wherein the pool of nucleotides in step (d) comprises (i) natural nucleotides; (ii) at least one modified nucleotide or (iii) modified nucleotides.
5. The process according to any one of clauses 1 to 4, wherein the at least one modified nucleotide comprises modification of the sugar moiety, modification of the nucleobase and/or modification of the backbone.
6. The process according to any one of clauses 1 to 5, wherein the at least one modified nucleotide comprises a modification at the 2′ position of the sugar moiety, a bicyclic sugar or a 4′-CH(CH3)-O-2′ group, or combinations thereof.
7. The process according to any one of clauses 1 to 6, wherein the at least one modified nucleotide comprises 2′-F, 2′-OMe, 2′-MOE, or 2′-amino.
8. The process according to any one of clauses 1 to 7, wherein the at least one modified nucleotide comprises a modified cytosine, 5-methylcytosine, 5-methyl pyrimidine, 7-deazaguanosine or an abasic nucleotide.
9. The process according to any one of clauses 1 to 8, wherein the at least one modified nucleotide comprises phosphorothioate, phosphoramidate, phosphorodiamidate or phosphorodithioate.
10. The process according to any one of clauses 1 to 9, wherein the at least one modified nucleotide comprises 1-methyl-pseudouridine, 5-methoxy-uracil, 1-ethyl-pseudouracil, pseudouracil, 1-methylpseudouracil, 5-methyl-cytidine, 5-methyl-cytosine, N6-methyladenosine or 7-methylguanosine.
11. The process according to any one of clauses 1 to 10, wherein the at least one modified nucleotide comprises 1-methyl-pseudouridine.
12. The process according to any one of clauses 1 to 4, wherein the at least one modified nucleotide comprises a PMO, a LNA, a c-Et, a PNA, a BNA, or a L-Ribonucleic acid.
13. The process according to any one of clauses 1 to 12, wherein the ligase in step (e) ligates a 3′ and/or a 5′ end of each segment polynucleotide or extended segment polynucleotide to each adjacent segment polynucleotide or adjacent extended segment polynucleotide to form the product polynucleotide strand.
14. The process according to any one of clauses 1 to 13, wherein the ligase in step (e) is capable of ligating RNA to RNA, DNA to DNA, RNA to DNA and/or DNA to RNA.
15. The process according to any one of clauses 1 to 14, wherein the ligase in step (e) is an RNA and/or a DNA ligase.
16. The process according to any one of clauses 1 to 15, wherein the polymerase in step (d) lacks strand-displacement activity.
17. The process according to any one of clauses 1 to 16, wherein the polymerase in step (d) lacks 5′ to 3′ exonuclease activity.
18. The process according to any one of clauses 1 to 17, where in the polymerase in step (d) has 3′ to 5′ exonuclease activity or lacks 3′ to 5′ exonuclease activity.
19. The process according to any one of clauses 1 to 18, wherein the polymerase is a DNA polymerase or an RNA polymerase.
20. The process according to any one of clauses 1 to 19, wherein the DNA polymerase or the RNA polymerase is an engineered or mutant DNA polymerase or RNA polymerase.
21. The process according to any one of clauses 1 to 20, wherein two or more of steps a) to g) occur concurrently, optionally steps d) and e) occur concurrently.
22. The process according to any one of clauses 1 to 21, wherein two or more of steps a) to g) occur sequentially, optionally steps d) and e) occur sequentially.
23. The process according to any one of clauses 1 to 22, wherein the template has a property that allows it to be separated from the single-stranded polynucleotide product.
24. The process according to clause 23, wherein the property that allows the template to be separated from the single-stranded polynucleotide product is that the template is attached to a support material.
25. The process according to clause 24, wherein the support material is a soluble support material, optionally wherein the support material is selected from the group consisting of polyethylene glycol, a soluble organic polymer, DNA, a protein, a dendrimer, a polysaccharide, an oligosaccharide, and a carbohydrate.
26. The process according to clause 24, wherein the support material is an insoluble support material, optionally wherein the support material is selected from the group consisting of: a glass bead, a polymeric bead, a fibrous support, a membrane, a streptavidin coated bead, cellulose and a reaction wall, optionally wherein the reaction wall is part of the reaction vessel.
27. The process according to any one of clauses 24 to 26, wherein multiple, repeated copies of the template are attached in a continuous manner via a single attachment point to the support material.
28. The process according to any one of clauses 23 to 27, wherein the property that allows the template to be separated from the product is the molecular weight of the template.
29. The process according to any one of clauses 1 to 28, wherein the process additionally comprises step h) recycling the template.
30. The process according to clause 29, wherein the process additionally comprises step i) repeating steps a) to g) or steps a) to h) with the recycled template.
31. The process according to any one of clauses 1 to 30, wherein the process is semi-continuous or continuous.
32. The process according to any one of clauses 1 to 31, wherein the template polynucleotide consists of a sequence complementary to the single-stranded polynucleotide product.
33. The process according to any one of clauses 1 to 32, wherein the single-stranded polynucleotide product is 3 to 30 nucleotides long, optionally 10 to 30 nucleotides long, 3 to 15 nucleotides long, 15 to 20 nucleotides long, 20 to 25 nucleotides long, or 20 to 30 nucleotides long.
34. The process according to any one of clauses 1 to 33, wherein the single-stranded polynucleotide product is 20 nucleotides long and the at least two segment polynucleotides comprise:
(i) a 5′ segment that is 7 nucleotides long and a 3′ segment that is 7 nucleotides long;
(ii) a 5′ segment that is 6 nucleotides long and a 3′ segment that is 6 nucleotides long;
(iii) a 5′ segment that is 5 nucleotides long and a 3′ segment that is 5 nucleotides long;
(iv) a 5′ segment that is 4 nucleotides long and a 3′ segment that is 4 nucleotides long; or
(v) a 5′ segment that is 3 nucleotides long and a 3′ segment that is 3 nucleotides long.
35. The process according to any one of clauses 1 to 34, wherein the single-stranded polynucleotide product is a gapmer.
36. The process according to any one of clauses 1 to 32, wherein the single-stranded polynucleotide product is 30 to 20,000 nucleotides long, optionally 30 to 10,000 nucleotides long, 30 to 5,000 nucleotides long, 30 to 1,000 nucleotides long, 30 to 500 nucleotides long, 30 to 400 nucleotides long, 30 to 300 nucleotides long, 30 to 200 nucleotides long, 30 to 100 nucleotides long, 30 to 50 nucleotides long, or 30 to 40 nucleotides long.
37. The process according to any one of clauses 1 to 36, wherein the single-stranded polynucleotide product is a DNA polynucleotide product, an RNA polynucleotide product, or a DNA and RNA hybrid polynucleotide product.
38. The process according to clause 37, wherein the RNA polynucleotide product is an mRNA.
39. The process according to any one of clauses 1 to 38, wherein the product is produced at gram, kilogram, or greater scale and/or the process is carried out in a reaction volume of at least 1 L.
40. The process according to any one of clauses 1 to 39, wherein the resulting single-stranded polynucleotide product is at least 80% pure, optionally wherein the single-stranded polynucleotide or oligonucleotide product is at least 90% pure, optionally wherein the single-stranded polynucleotide or oligonucleotide product is at least 95% pure, optionally wherein the single-stranded polynucleotide or oligonucleotide product is at least 98% pure.
41. A process for producing a double-stranded polynucleotide product, wherein two complementary single-stranded polynucleotides produced by the process of any one of clauses 1 to 40 are mixed under conditions to allow annealing.
42. A process for producing a double-stranded polynucleotide product having at least one modified nucleotide residue, the process comprising:
a) providing a template polynucleotide comprising a sequence complementary to a single-stranded polynucleotide product;
b) providing a pool of polynucleotides comprising at least two segment polynucleotides;
c) contacting the template polynucleotide of step (a) with the pool of polynucleotides of step (b) under conditions to allow annealing of the at least two segment polynucleotides to the template polynucleotide, wherein at least one sequence gap is formed between the at least two segment polynucleotides;
d) providing a pool of nucleotides and a polymerase, and extending at least one segment polynucleotide using the pool of nucleotides and the polymerase, to fill in the at least one sequence gap;
e) ligating segment polynucleotides and/or extended segment polynucleotides using a ligase to form the single-stranded polynucleotide product;
f) optionally, changing the conditions to denature the annealed template and any impurities, and separating the impurities;
g) changing the conditions to denature the annealed template and the single-stranded polynucleotide product, and separating the single-stranded polynucleotide product; and
h) using the single-stranded polynucleotide product as the template in step a) and repeating steps a) to e) to produce the double-stranded polynucleotide product.
43. The process according to clause 42, wherein the double-stranded polynucleotide product is purified.
44. The process according to any one of clauses 41 to 43, wherein the double-stranded polynucleotide product is an siRNA.
45. The process according to any one of clauses 1 to 44, wherein the polynucleotide product is a therapeutic polynucleotide product.
46. The process according to clause 37 or 38, wherein the RNA polynucleotide product comprises a sequence that encodes one or more immunogens.
47. The process according to clause 46, wherein the immunogens are selected from a respiratory syncytial virus (RSV) immunogen, an Epstein-Barr virus glycoprotein immunogen, a cytomegalovirus glycoprotein immunogen, a coronavirus spike polypeptide immunogen, an influenza virus immunogen, a Varicella zoster virus glycoprotein immunogen, a human papillomavirus 16 (HPV16) E6 immunogen, a HPV 16 E7 immunogen, or a flavivirus immunogen.
48. The process according to clause 46 or 47, wherein the immunogens are selected from a coronavirus spike protein, an influenza antigen, and RSV antigen such as protein f or protein g.

### EXAMPLES

### Abbreviations

HPLC high performance liquid chromatography
LCMS liquid chromatography mass spectrometry
SEC size exclusion chromatography
TEAA triethylammonium acetate
PO phosphodiester
PS phosphorothioate
* phosphorothioate
/3Phos/ 3′ Phosphate group
/5Phos/ 5′ Phosphate group
/Me-dC/ 5-Methylcytosine
/5Biosg/ 5′ Biotin
EDTA ethylenediaminetetraacetic acid
dATP deoxyadenosine triphosphate
dCTP deoxycytidine triphosphate
dGTP deoxyguanosine triphosphate
dTTP deoxythymidine triphosphate
dATPαS 2′-deoxyadenosine-5′-(α-thio)-triphosphate
dCTPαS 2′-deoxycytidine-5′-(α-thio)-triphosphate
dGTPαS 2′-deoxyguanosine-5′-(α-thio)-triphosphate
dTTPαS 2′-deoxythymidine-5′-(α-thio)-triphosphate
ATP adenosine triphosphate
MgCl₂ magnesium chloride
Tris 2-Amino-2-(hydroxymethyl)propane-1,3-diol
TBuAA tributylammonium acetate
2′MOE 2′-0-methoxy-ethyl
2′OMe 2′-0-Methyl
2′F 2′ Fluoro
LNA locked nucleic acid
CTP cytidine triphosphate
GTP guanosine triphosphate
UTP uridine triphosphate
mA 2′-0-methyl adenosine
mC 2′-0-methyl cytidine
mG 2′-0-methyl guanosine
mU 2′-0-methyl uridine
rA 2′-hydroxyl adenosine
rC 2′-hydroxyl cytidine
rG 2′-hydroxyl guanosine
rU 2′-hydroxyl adenosine
fA 2′ fluoro adenosine
fC 2′ fluoro cytidine
fG 2′ fluoro guanosine
fU 2′ fluoro uridine
eA 2′-0-methoxy-ethyl adenosine
eC 2′-0-methoxy-ethyl 5-methylcytosine
eG 2′-0-methoxy-ethyl guanosine
eT 2′-0-methoxy-ethyl thymidine
ψTP Pseudouridine-5′-triphosphate
m1ψTP N1-Methylpseudouridine-5′-triphosphate
ψ Pseudouridine
m1ψ N1-Methylpseudouridine
ATPαS adenosine-5′-(α-thio)-triphosphate
CTPαS cytidine-5′-(α-thio)-triphosphate
GTPαS guanosine-5′-(α-thio)-triphosphate
UTPαS uridine-(α-thio)-triphosphate

### Example 1: 5 bp 2′H Oligonucleotide Synthesis from 10 bp 2′H Oligonucleotide Primer Gap Filling Between a 27 bp 2′H Oligonucleotide 3′Block on a 42-Bp 2′H Oligonucleotide Template

Aim: Demonstrate gap filling between unmodified DNA oligonucleotide segments using unmodified deoxyribonucleoside triphosphates to show general applicability of the technology.

Oligonucleotides (SEQ ID NOs: 1 to 38) were acquired from commercially available sources. Primer-extending polymerases (SEQ ID NOs: 39 to 43) were acquired from commercially available sources and used directly.

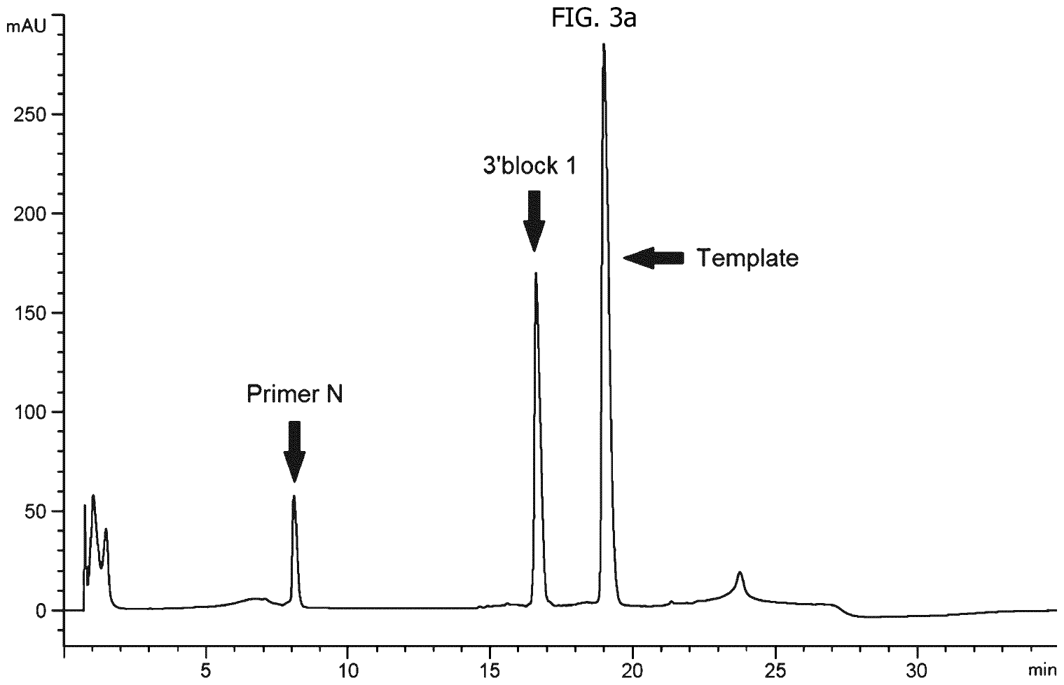

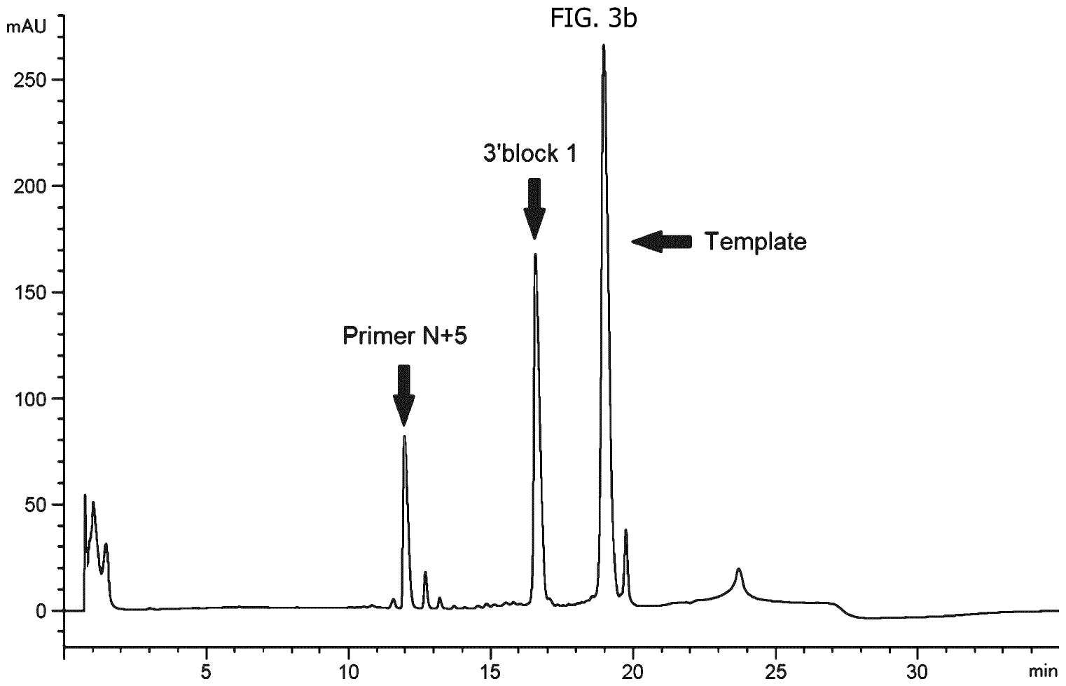

Reactions were set up as per Table 1. Oligonucleotides contained within the reactions were annealed by heating to 95° C. and cooling to 15° C. at 0.1° C./sec. Polymerase was then added to commence the reaction. Reactions were incubated at 25° C. for 2 hours. Reactions were then quenched by addition of 50 μL 100 mM EDTA, reactions were then buffer exchanged in to 10 mM EDTA using SEC. Reactions were then analysed by HPLC for the presence of SEQ ID NO: 11. FIG. 3a shows a chromatogram of reaction starting materials. FIG. 3b shows a chromatogram of a product forming reaction.

TABLE 1

| | Final concentration | Volume per |
|---|---|---|
| | in reaction (mM) | reaction (μL) |
| Polymerase - SEQ ID NOs: 39 to 44 | | 1.0 |
| Template: (2′H)- SEQ ID NO: 1 | 0.04 | 12.5 |
| 5′primer (2′H) - SEQ ID NO: 2 | 0.04 | 2.0 |
| 3′block 1 (2′H) - SEQ ID NO: 3 | 0.04 | 1.5 |
| dATP | 0.5 | 2.5 |
| dCTP | 0.5 | 2.5 |
| dGTP | 0.5 | 2.5 |
| dTTP | 0.5 | 2.5 |
| Tris (pH 7.5) | 50 | 2.5 |
| MgCl₂ | 10 | 0.5 |
| Water | | 20.0 |
| Final reaction volume (μL) | | 50 |

Results from reactions:

TABLE 2

| | SED ID | Conversion (%) |
|---|---|---|
| Gene name | NO: | to product* |
| Wild-type Escherichia phage T7 polymerase - ‘Pol1’ | 39 | 17.01 |
| Wild-type Sulfolobus solfataricus polymerase - ‘Pol2’ | 40 | 54.42 |
| Mutant Thermococcus sp. (strain 9oN-7) polymerase - ‘Pol3’ | 41 | 67.75 |
| Wild-type Enterobacteria phage T4 polymerase - ‘Pol4’ | 42 | 81.21 |
| Wild-type Thermus aquaticus polymerase - ‘Pol5’ | 43 | 56.57 |

*Conversion (%) to product - % area of 5′-Primer SEQ ID NO: 2 + SEQ ID NOs: 7 to 38 peaks vs. SEQ ID NO: 11 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 35-71% buffer B was run over 18 minutes before being stepped up to 95% buffer B for 7 minutes.
Buffer A: 100 mM TEAA, pH 7
Buffer B: 100 mM TEAA, pH 7 in 25% Acetonitrile/75% H₂O

Conclusion: Genetically diverse set of primer-extending polymerases are shown to perform the base filling between two flanking oligonucleotides. Top example with high product formation (greater than 80% by area) and low (less than 20% by area) incomplete reaction or side product formation.

### Example 2: One-Pot 5 bp 2′H Oligonucleotide Synthesis from 10 bp 2′H Oligonucleotide Primer Gap Filling Between a 27 bp 2′H Oligonucleotide 3′Block on a 42-Bp 2′H Oligonucleotide Template and Ligation to Synthesis 42-Bp Product

Aim: Demonstrate gap filling and ligation with unmodified DNA oligonucleotide segments and unmodified deoxyribonucleoside triphosphates to show general applicability of the technology.

Oligonucleotides (SEQ ID NOs: 1 to 38) were acquired from commercially available sources. Primer-extending polymerases (SEQ ID NOs: 39 to 43) were acquired from commercially available sources and used directly. Double-stranded ligase (SEQ ID NO: 45) was acquired from a commercially available source and used directly.

Reactions were set up as per Table 3. Oligonucleotides contained within the reactions were annealed by heating to 95° C. and cooling to 15° C. at 0.1° C./sec. Polymerase and ligase were then added to commence the reaction. Reactions were incubated at 25° C. for 2 hours. Reactions were then quenched by addition of 50 μL 100 mM EDTA, reactions were then buffer exchanged in to 10 mM EDTA using SEC. Reactions were then analysed by HPLC for the presence of SEQ ID NO: 38.

TABLE 3

| | Final concentration | Volume per reaction |
|---|---|---|
| | in reaction (mM) | (μL) |
| Polymerase - SEQ ID NO: 39 to 44 | | 1.0 |
| Ligase - SEQ ID NO: 45 | | 2.5 |
| Template: (2′H)- SEQ ID NO: 1 | 0.04 | 12.5 |
| 5′primer (2′H) - SEQ ID NO: 2 | 0.04 | 2.0 |
| 3′block 1 (2′H) - SEQ ID NO: 3 | 0.04 | 1.5 |
| dATP | 0.5 | 2.5 |
| dCTP | 0.5 | 2.5 |
| dGTP | 0.5 | 2.5 |
| dTTP | 0.5 | 2.5 |
| ATP | 1 | 5 |
| Tris (pH 7.5) | 50 | 2.5 |
| MgCl₂ | 10 | 0.5 |
| Water | | 12.5 |
| Final reaction volume (uL) | | 50 |

Results from reactions:

TABLE 4

| | SED ID | Conversion (%) |
|---|---|---|
| Gene name | NO: | to product* |
| Wild-type Escherichia | 39 | 5.18 |
| phage T7 polymerase - ‘Pol1’ | | |
| Wild-type Sulfolobus solfataricus | 40 | 81.22 |
| polymerase - ‘Pol2’ | | |
| Mutant Thermococcus sp. | 41 | 88.71 |
| (strain 9oN-7) polymerase - ‘Pol3’ | | |
| Wild-type Enterobacteria | 42 | 95.16 |
| phage T4 polymerase - ‘Pol4’ | | |
| Wild-type Thermus aquaticus | 43 | 41.19 |
| polymerase - ‘Pol5’ | | |

*Conversion (%) to product - % area of SEQ ID NO: 2 + SEQ ID NOs: 7 to 38 peaks vs. SEQ ID NO: 38 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 35-71% buffer B was run over 18 minutes before being stepped up to 95% buffer B for 7 minutes.
Buffer A: 100 mM TEAA, pH 7
Buffer B: 100 mM TEAA, pH 7 in 25% Acetonitrile/75% H₂O

Conclusion: Genetically diverse set of primer-extending polymerases are shown to perform the base filling between two flanking oligonucleotides, and ligase is able to perform tandem reaction to afford full-length oligonucleotide. Top example with high product formation (greater than 95% by area) and low (less than 5% by area) incomplete reaction or side product formation.

### Example 3: 6 bp 2′H Oligonucleotide Synthesis from 10 bp 2′OH Oligonucleotide Primer Gap Filling Between a 26 bp 2′H Oligonucleotide 3′Block on a 42-Bp 2′H Oligonucleotide Template

Aim: Demonstrate gap filling between an RNA oligonucleotide segment (primer) and unmodified DNA oligonucleotide segment (stopper/block) using unmodified deoxyribonucleoside triphosphates to show application of the technology to a sugar group (2′OH) commonly appearing in oligonucleotide therapeutics.

Oligonucleotides (SEQ ID NOs: 1 to 38) were acquired from commercially available sources. Primer-extending polymerases (SEQ ID NOs: 40, 41 and 42) were acquired from commercially available sources and used directly.

Reactions were set up as per Table 5. Oligonucleotides contained within the reactions were annealed by heating to 95° C. and cooling to 15° C. at 0.1° C./sec. Polymerase was then added to commence the reaction. Reactions were incubated at 25° C. for 2 hours. Reactions were then quenched by addition of 25 μL 100 mM EDTA, reactions were then buffer exchanged in to 10 mM EDTA using SEC. Reactions were then analysed by HPLC for the presence of SEQ ID NO: 12.

TABLE 5

| | | Volume |
|---|---|---|
| | Final | per |
| | concentration in | reaction |
| | reaction (mM) | (μL) |
| Polymerase - SEQ ID NOs: 40, 41 and 42 | | 1.3 |
| Template: (2′H)- SEQ ID NO: 1 | 0.04 | 6.3 |
| 5′primer (2′OH) - SEQ ID NO: 2 | 0.04 | 1.0 |
| 3′block 2 (2′H) - SEQ ID NO: 4 | 0.04 | 1.0 |
| dATP | 0.5 | 1.3 |
| dCTP | 0.5 | 1.3 |
| dGTP | 0.5 | 1.3 |
| dTTP | 0.5 | 1.3 |
| Tris (pH 7.5) | 50 | 1.3 |
| MgCl₂ | 10 | 0.2 |
| Water | | 9.0 |
| Final reaction volume (μL) | | 25 |

Results from reactions:

TABLE 6

| | SED ID | Conversion (%) |
|---|---|---|
| Gene name | NO: | to product* |
| Wild-type Sulfolobus solfataricus | 40 | 24.44 |
| polymerase - ‘Pol2’ | | |
| Wild-type Enterobacteria | 41 | 8.20 |
| phage T4 polymerase - ‘Pol4’ | | |
| Wild-type Thermococcus kodakarens | 42 | 11.34 |
| polymerase - ‘Pol6’ | | |

*Conversion (%) to product - % area of SEQ ID NO: 2 + SEQ ID NOs: 7 to 38 peaks vs. SEQ ID NO: 12 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 35-71% buffer B was run over 18 minutes before being stepped up to 95% buffer B for 7 minutes.
Buffer A: 100 mM TEAA, pH 7
Buffer B: 100 mM TEAA, pH 7 in 25% Acetonitrile/75% H₂O

Conclusion: Proof of concept that primer-extending polymerases can be used to perform the base filling between two flanking oligonucleotides extending from a 2′-OH sugar modified 5′primer. Top example polymerase with product formation greater than 24% by area.

### Example 4: 15 bp 2′H Oligonucleotide Synthesis from 10 bp 2′H Oligonucleotide Primer Gap Filling Between a 17 bp 2′H 5-Methylcytosine Base Oligonucleotide 3′Block on a 42-Bp 2′H Oligonucleotide Template

Aim: Demonstrate gap filling between one modified oligonucleotide segment and one unmodified oligonucleotide segment using unmodified deoxyribonucleoside triphosphates to show application of the technology to a base modification commonly appearing in oligonucleotide therapeutics.

Oligonucleotides (SEQ ID NOs: 1 to 38) were acquired from commercially available sources. Primer-extending polymerases (SEQ ID NOs: 40, 42 and 44) were acquired from commercially available sources and used directly.

Reactions were set up as per Table 7. Oligonucleotides contained within the reactions were annealed by heating to 95° C. and cooling to 15° C. at 0.1° C./sec. Polymerase was then added to commence the reaction. Reactions were incubated at 25° C. for 2 hours. Reactions were then quenched by addition of 25 μL 100 mM EDTA, reactions were then buffer exchanged in to 10 mM EDTA using SEC. Reactions were then analysed by HPLC for the presence of SEQ ID NO: 21.

TABLE 7

| | Final | Volume |
|---|---|---|
| | concentration in | per reaction |
| | reaction (mM) | (μL) |
| Polymerase - SEQ ID NOs: 40, 42 and | | 1.3 |
| 44 | | |
| Template: (2′H)- SEQ ID NO: 1 | 0.04 | 6.3 |
| 5′primer (2′H) - SEQ ID NO: 2 | 0.04 | 1.0 |
| 3′block 3 (2′H) - SEQ ID NO: 5 | 0.04 | 1.0 |
| dATP | 0.5 | 1.3 |
| dCTP | 0.5 | 1.3 |
| dGTP | 0.5 | 1.3 |
| dTTP | 0.5 | 1.3 |
| Tris (pH 7.5) | 50 | 1.3 |
| MgCl₂ | 10 | 0.2 |
| Water | | 9.0 |
| Final reaction volume (μL) | | 25 |

Results from reactions:

TABLE 8

| | SED ID | Conversion (%) |
|---|---|---|
| Gene name | NO: | to product* |
| Wild-type Sulfolobus solfataricus | 40 | 59.60 |
| polymerase - ‘Pol2’ | | |
| Wild-type Enterobacteria | 42 | 76.61 |
| phage T4 polymerase - ‘Pol4’ | | |
| Wild-type Thermococcus kodakarens | 44 | 79.10 |
| polymerase - ‘Pol6’ | | |

*Conversion (%) to product - % area of SEQ ID NO: 2 + SEQ ID NOs: 7 to 38 peaks vs. SEQ ID NO: 21 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 35-71% buffer B was run over 18 minutes before being stepped up to 95% buffer B for 7 minutes.
Buffer A: 100 mM TEAA, pH 7
Buffer B: 100 mM TEAA, pH 7 in 25% Acetonitrile/75% H₂O

Conclusion: Proof of concept that primer-extending polymerases can be used to perform the base filling between two flanking oligonucleotides with at least base modification contained within the flanking oligonucleotides. Top example polymerase with product formation greater than 79% by area.

### Example 5: 6 bp 2′H Fully PS Modified Oligonucleotide Synthesis from 10 bp 2′H Oligonucleotide Primer Gap Filling Between a 26 bp 2′H Oligonucleotide 3′Block on a 42-Bp 2′H Oligonucleotide Template

Aim: Demonstrate gap filling between unmodified oligonucleotide segments with modified deoxyribonucleoside triphosphates to show application of the technology to backbone modification commonly appearing in oligonucleotide therapeutics.

Oligonucleotides (SEQ ID NOs: 1 to 38) were acquired from commercially available sources. Primer-extending polymerases (SEQ ID NOs: 40, 41 and 42) were acquired from commercially available sources and used directly.

Reactions were set up as per Table 9. Oligonucleotides contained within the reactions were annealed by heating to 95° C. and cooling to 15° C. at 0.1° C./sec. Polymerase was then added to commence the reaction. Reactions were incubated at 25° C. for 2 hours. Reactions were then quenched by addition of 5 μL 500 mM EDTA, reactions were then buffer exchanged into water using SEC. Reactions were then analysed by HPLC for the presence of SEQ ID NOs: 12.

TABLE 9

| | Final | Volume |
|---|---|---|
| | concentration in | per reaction |
| | reaction (mM) | (μL) |
| Polymerase - SEQ ID NO 40, 41 and | | 1.0 |
| 42 | | |
| Template: (2′H)- SEQ ID NO 1 | 0.04 | 12.5 |
| 5′primer (2′H) - SEQ ID NO 2 | 0.04 | 1.8 |
| 3′block 2 (2′H) - SEQ ID NO 4 | 0.04 | 1.9 |
| dATPαS | 0.5 | 2.5 |
| dCTPαs | 0.5 | 2.5 |
| dGTPαS | 0.5 | 2.5 |
| dTTPαS | 0.5 | 2.5 |
| Tris (pH 7.5) | 50 | 2.5 |
| MgCl₂ | 10 | 0.5 |
| Water | | 19.8 |
| Final reaction volume (μL) | | 50 |

Results from reactions:

TABLE 10

| | SED | Conversion |
|---|---|---|
| | ID | (%) to |
| Gene name | NO: | product* |
| Wild-type Sulfolobus solfataricus | 40 | 8.25 |
| polymerase - ‘Pol2’ | | |
| Mutant Thermococcus sp. | 41 | 30.54 |
| (strain 9oN-7) polymerase - ‘Pol3’ | | |
| Wild-type Enterobacteria | 42 | 33.58 |
| phage T4 polymerase - ‘Pol4’ | | |

*Conversion (%) to product - % area of SEQ ID NO: 2 + SEQ ID NOs: 7 to 38 peaks vs. SEQ ID NO: 12 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 35-71% buffer B was run over 18 minutes before being stepped up to 95% buffer B for 7 minutes.
Buffer A: 100 mM TEAA, pH 7
Buffer B: 100 mM TEAA, pH 7 in 25% Acetonitrile/75% H₂O

Conclusion: Proof of concept that primer-extending polymerases can be used to perform backbone modified base filling between two flanking oligonucleotides. Filling the gap by synthesizing a completely phosphorothioate modified backbone. Top example polymerase with product formation greater than 33% by area.

### Example 6: 6 bp 2′H Deoxyguanosine PS Modified Oligonucleotide Synthesis from 10 bp 2′H Oligonucleotide Primer Gap Filling Between a 26 bp 2′H Oligonucleotide 3′Block on a 42-Bp 2′H Oligonucleotide Template

Aim: Demonstrate gap filling between unmodified oligonucleotide segments using modified deoxyguanosine triphosphate, but unmodified deoxyadenosine triphosphate, unmodified deoxycytidine triphosphate and unmodified deoxythymidine triphosphate, to show application of the technology to modular backbone modification commonly appearing in oligonucleotide therapeutics.

Oligonucleotides (SEQ ID NOs: 1 to 38) were acquired from commercially available sources. Primer-extending polymerases (SEQ ID NOs: 40, 41 and 42) were acquired from commercially available sources and used directly.

Reactions were set up as per Table 11. Oligonucleotides contained within the reactions were annealed by heating to 95° C. and cooling to 15° C. at 0.1° C./sec. Polymerase was then added to commence the reaction. Reactions were incubated at 25° C. for 2 hours. Reactions were then quenched by addition of 5 μL 500 mM EDTA, reactions were then buffer exchanged into water using SEC. Reactions were then analysed by HPLC for the presence of SEQ ID NO: 12.

TABLE 11

| | Final | Volume |
|---|---|---|
| | concentration in | per reaction |
| | reaction (mM) | (μL) |
| Polymerase - SEQ ID NOs: 40, 41 and | | 1.0 |
| 42 | | |
| Template: (2′H)- SEQ ID NO: 1 | 0.04 | 12.5 |
| 5′primer (2′H) - SEQ ID NO: 2 | 0.04 | 1.8 |
| 3′block 2 (2′H) - SEQ ID NO: 4 | 0.04 | 1.9 |
| dATP | 0.5 | 2.5 |
| dCTP | 0.5 | 2.5 |
| dGTPαS | 0.5 | 2.5 |
| dTTP | 0.5 | 2.5 |
| Tris (pH 7.5) | 50 | 2.5 |
| MgCl₂ | 10 | 0.5 |
| Water | | 19.8 |
| Final reaction volume (μL) | | 50 |

Results from reactions:

TABLE 12

| | | Conversion |
|---|---|---|
| | SED ID | (%) to |
| Gene name | NO: | product* |
| Wild-type Sulfolobus solfataricus | 40 | 59.39 |
| polymerase - ‘Pol2’ | | |
| Mutant Thermococcus sp. | 41 | 43.07 |
| (strain 9oN-7) polymerase - ‘Pol3’ | | |
| Wild-type Enterobacteria | 42 | 89.77 |
| phage T4 polymerase - ‘Pol4’ | | |

*Conversion (%) to product - % area of SEQ ID NO: 2 + SEQ ID NOs: 7 to 38 peaks vs. SEQ ID NO: 12 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 35-71% buffer B was run over 18 minutes before being stepped up to 95% buffer B for 7 minutes.
Buffer A: 100 mM TEAA, pH 7
Buffer B: 100 mM TEAA, pH 7 in 25% Acetonitrile/75% H₂O

Conclusion: Proof of concept that primer-extending polymerases can be used to perform specific backbone modified base filling between two flanking oligonucleotides. Filling the gap by synthesizing phosphorothioate modified backbone linkage between desired bases. Top example polymerase with product formation greater than 89% by area.

### Example 7: 6 bp 2′H Deoxythymidine PS Modified Oligonucleotide Synthesis from 10 bp 2′H Oligonucleotide Primer Gap Filling Between a 26 bp 2′H Oligonucleotide 3′Block on a 42-Bp 2′H Oligonucleotide Template

Aim: Demonstrate gap filling between unmodified oligonucleotide segments using modified deoxythymidine triphosphate, but unmodified deoxyadenosine triphosphate, unmodified deoxycytidine triphosphate and unmodified deoxyguanosine triphosphate, to show application of the technology to modular backbone modification commonly appearing in oligonucleotide therapeutics.

Oligonucleotides (SEQ ID NOs: 1 to 38) were acquired from commercially available sources. Primer-extending polymerases (SEQ ID NOs: 40, 41 and 42) were acquired from commercially available sources and used directly.

Reactions were set up as per Table 13. Oligonucleotides contained within the reactions were annealed by heating to 95° C. and cooling to 15° C. at 0.1° C./sec. Polymerase was then added to commence the reaction. Reactions were incubated at 25° C. for 2 hours. Reactions were then quenched by addition of 5 μL 500 mM EDTA, reactions were then buffer exchanged into water using SEC. Reactions were then analysed by HPLC for the presence of SEQ ID NO: 12.

TABLE 13

| | Final | Volume |
|---|---|---|
| | concentration in | per reaction |
| | reaction (mM) | (μL) |
| Polymerase - SEQ ID NOs: 40, 41 and | | 1.0 |
| 42 | | |
| Template: (2′H)- SEQ ID NO: 1 | 0.04 | 12.5 |
| 5′primer (2′H) - SEQ ID NO: 2 | 0.04 | 1.8 |
| 3′block 2 (2′H) - SEQ ID NO: 4 | 0.04 | 1.9 |
| dATP | 0.5 | 2.5 |
| dCTP | 0.5 | 2.5 |
| dGTP | 0.5 | 2.5 |
| dTTPαS | 0.5 | 2.5 |
| Tris (pH 7.5) | 50 | 2.5 |
| MgCl₂ | 10 | 0.5 |
| Water | | 19.8 |
| Final reaction volume (μL) | | 50 |

Results from reactions:

TABLE 14

| | SED ID | Conversion (%) |
|---|---|---|
| Gene name | NO: | to product* |
| Wild-type Sulfolobus solfataricus | 40 | 53.39 |
| polymerase - ‘Pol2’ | | |
| Mutant Thermococcus sp. | 41 | 29.36 |
| (strain 9oN-7) polymerase - ‘Pol3’ | | |
| Wild-type Enterobacteria | 42 | 78.44 |
| phage T4 polymerase - ‘Pol4’ | | |

*Conversion (%) to product - % area of SEQ ID NO: 2 + SEQ ID NOs: 7 to 38 peaks vs. SEQ ID NO: 12 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 35-71% buffer B was run over 18 minutes before being stepped up to 95% buffer B for 7 minutes.
Buffer A: 100 mM TEAA, pH 7
Buffer B: 100 mM TEAA, pH 7 in 25% Acetonitrile/75% H₂O

Conclusion: Proof of concept that primer-extending polymerases can be used to perform specific backbone modified base filling between two flanking oligonucleotides. Filling the gap by synthesizing phosphorothioate modified backbone linkage between desired bases. Top example polymerase with product formation greater than 89% by area.

### Example 8: One-Pot 15 bp 2′H Oligonucleotide Synthesis from 10 bp 2′H Oligonucleotide Primer Gap Filling Between a 17 bp 2′H 5-Methylcytosine Base Oligonucleotide 3′Block on a 42-Bp 2′H Oligonucleotide Template Oligonucleotide Template and Ligation to Synthesis 42-Bp Product

Aim: Demonstrate gap filling and ligation with one modified oligonucleotide segment and one unmodified oligonucleotide segment and unmodified deoxyribonucleoside triphosphates to show application of the technology to base modification commonly appearing in oligonucleotide therapeutics.

Oligonucleotides (SEQ ID NOs: 1 to 38) were acquired from commercially available sources. Primer extending polymerases (SEQ ID NOs: 40, 42 and 44) were acquired from commercially available sources and used directly. Double-stranded ligase (SEQ ID NO: 45) was acquired from a commercially available source and used directly.

Reactions were set up as per Table 15. Oligonucleotides contained within the reactions were annealed by heating to 95° C. and cooling to 15° C. at 0.1° C./sec. Polymerase and ligase was then added to commence the reaction. Reactions were incubated at 25° C. for 2 hours. Reactions were then quenched by addition of 25 μL 100 mM EDTA, reactions were then buffer exchanged in to 10 mM EDTA using SEC. Reactions were then analysed by HPLC for the presence of SEQ ID NO: 38.

TABLE 15

| | Final | Volume |
|---|---|---|
| | concentration in | per reaction |
| | reaction (mM) | (μL) |
| Polymerase - SEQ ID NO: 40, 42 and | | 1.3 |
| 44 | | |
| Ligase - SEQ ID NO: 45 | | 0.5 |
| Template: (2′H)- SEQ ID NO: 1 | 0.04 | 6.3 |
| 5′primer (2′H) - SEQ ID NO: 2 | 0.04 | 1.0 |
| 3′block 3 (2′H) - SEQ ID NO: 5 | 0.04 | 1.0 |
| dATP | 0.5 | 1.3 |
| dCTP | 0.5 | 1.3 |
| dGTP | 0.5 | 1.3 |
| dTTP | 0.5 | 1.3 |
| ATP | 1 | 2.5 |
| Tris (pH 7.5) | 50 | 1.3 |
| MgCl₂ | 10 | 0.2 |
| Water | | 6.0 |
| Final reaction volume (μL) | | 25 |

Results from reactions:

TABLE 16

| | SED ID | Conversion (%) |
|---|---|---|
| Gene name | NO: | to product* |
| Wild-type Sulfolobus solfataricus | 40 | 60.77 |
| polymerase - ‘Pol2’ | | |
| Wild-type Enterobacteria | 42 | 81.33 |
| phage T4 polymerase - ‘Pol4’ | | |
| Wild-type Thermococcus kodakarens | 44 | 79.90 |
| polymerase - ‘Pol6’ | | |

*Conversion (%) to product - % area of SEQ ID NO: 2 + SEQ ID NO: 5 + SEQ ID NO: 7 to 38 peaks vs. SEQ ID NO: 38 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 35-71% buffer B was run over 18 minutes before being stepped up to 95% buffer B for 7 minutes.

Conclusion: Proof of concept that primer-extending polymerases can be used to perform the base filling between two flanking oligonucleotides with at least one base modification contained within one of the flanking oligonucleotides. Also, that a ligase is able to perform tandem ligation reaction to afford full-length oligonucleotide from the synthesized fragment. Top example polymerase and ligase with product formation greater than 81% by area.

### Example 9: One-Pot 6 bp 2′F Modified Oligonucleotide Synthesis from 10 bp 2′H Oligonucleotide Primer Gap Filling Between a 26 bp 2′H Oligonucleotide 3′Block 2′H on a 45-Bp 2′H Oligonucleotide Template and Ligation to Synthesis 42-Bp Product

Aim: Demonstrate gap filling between unmodified oligonucleotide segments using unmodified deoxythymidine triphosphate, but modified deoxyadenosine triphosphate, modified deoxycytidine triphosphate and modified deoxyguanosine triphosphate, to show application of the technology to sugar modification commonly appearing in oligonucleotide therapeutics.

Oligonucleotides (SEQ ID NOs: 1 to 38 & 46 to 54) were acquired from commercially available sources. Primer-extending polymerases (SEQ ID NO: 67, 69, 73, 77 and 87) were acquired from commercially available sources and used directly. Double-stranded ligase (SEQ ID NO: 45) was acquired from a commercially available source and used directly.

Reactions were set up as per Table 17. Oligonucleotides contained within the reactions were annealed by heating to 95° C. and cooling to 15° C. at 0.1° C./sec. Polymerase and ligase were then added to commence the reaction. Reactions were incubated at 25° C. for 4 hours. Reactions were then quenched by addition of 60 μL 100 mM EDTA, reactions were then buffer exchanged into water using SEC. Reactions were then analysed by HPLC for the presence of SEQ ID NO: 38.

TABLE 17

| | Final | Volume |
|---|---|---|
| | concentration in | per reaction |
| | reaction (mM) | (μL) |
| Polymerase - SEQ ID NO: 67, 69, 73, | | 1.0 |
| 77 and 87) | | |
| RNAse inhibitor | | 1.0 |
| Ligase - SEQ ID NO: 45 | | 4.0 |
| Template XXL: (2′H) - SEQ ID NO: 46 | 0.04 | 0.8 |
| 5′primer (2′H) - SEQ ID NO: 2 | 0.04 | 0.8 |
| 3′block 2 (2′H) - SEQ ID NO: 4 | 0.04 | 0.8 |
| 2′-F-dATP | 0.5 | 1.0 |
| 2′-F-dGTP | 0.5 | 1.0 |
| 2′-F-dCTP | 0.5 | 1.0 |
| dTTP | 0.5 | 1.0 |
| ATP | 1 | 2.0 |
| Tris (pH 7.5) | 50 | 1.0 |
| MgCl₂ | 10 | 0.2 |
| KCl | 50 | 0.5 |
| Water | | 3.9 |
| Final reaction volume (μL) | | 20 |

Results from reactions:

TABLE 18

| | SED | Conversion |
|---|---|---|
| | ID | (%) to |
| Gene name | NO: | product* |
| Mutant Thermococcus kodakarensis | 67 | 72.16 |
| polymerase - ‘Kod-V2’ | | |
| Mutant Thermococcus litoralis | 69 | 90.10 |
| polymerase - ‘Vent-V1’ | | |
| Mutant Thermococcus gorgonarius | 73 | 75.43 |
| polymerase - ‘Tgo-V5’ | | |
| Mutant Thermococcus sp. | 77 | 77.19 |
| polymerase - ‘9n-V4’ | | |
| Mutant Thermococcus gorgonarius | 87 | 71.60 |
| polymerase - ‘Tgo-V12’ | | |

*Conversion (%) to product - % area of SEQ ID NO: 2 + SEQ ID NO: 4 + SEQ ID NO: 7 to 38 peaks vs. SEQ ID NO: 38 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform specific sugar modified base filling between two flanking oligonucleotides. Filling the gap by incorporating 2′-Fluoro modifications at desired base. Also, that a ligase is able to perform tandem ligation reaction to afford full-length oligonucleotide from the synthesized fragment. Top example polymerase and ligase with product formation greater than 90.1% by area.

### Example 10: One-Pot 6 bp 2′H Deoxycytidine Me-dC Modified Oligonucleotide Synthesis from 10 bp 2′H Oligonucleotide Primer Gap Filling Between a 26 bp 2′H Oligonucleotide 3′Block 2′H on a 45-Bp 2′H Oligonucleotide Template and Ligation to Synthesis 42-Bp Product

Aim: Demonstrate gap filling between unmodified oligonucleotide segments using modified deoxycytidine triphosphate, but unmodified deoxyadenosine triphosphate, unmodified deoxyguanosine triphosphate and unmodified deoxythymidine triphosphate, to show application of the technology to modular base modification commonly appearing in oligonucleotide therapeutics.

Oligonucleotides (SEQ ID NOs: 1 to 38 & 46 to 54) were acquired from commercially available sources. Primer-extending polymerases (SEQ ID NO: 42, 44 and 86) were acquired from commercially available sources and used directly. Double-stranded ligase (SEQ ID NO: 45) was acquired from a commercially available source and used directly.

Reactions were set up as per Table 19. Oligonucleotides contained within the reactions were annealed by heating to 95° C. and cooling to 15° C. at 0.1° C./sec. Polymerase and ligase were then added to commence the reaction. Reactions were incubated at 25° C. for 4 hours. Reactions were then quenched by addition of 60 μL 100 mM EDTA, reactions were then buffer exchanged into water using SEC. Reactions were then analysed by HPLC for the presence of SEQ ID NO: 38.

TABLE 19

| | Final | Volume |
|---|---|---|
| | concentration | per reaction |
| | in reaction (mM) | (μL) |
| Polymerase - SEQ ID NO: | | 1.0 |
| 42, 44 and 86 | | |
| RNAse inhibitor | | 1.0 |
| Ligase - SEQ ID NO: 45 | | 1.0 |
| Template XXL: (2′H) - | 0.04 | 0.8 |
| SEQ ID NO: 46 | | |
| 5′primer (2′H) - | 0.04 | 0.8 |
| SEQ ID NO: 2 | | |
| 3′block 2 (2′H) - | 0.04 | 0.8 |
| SEQ ID NO: 4 | | |
| dATP | 0.5 | 1.0 |
| dGTP | 0.5 | 1.0 |
| 5-me-dCTP | 0.5 | 1.0 |
| dTTP | 0.5 | 1.0 |
| ATP | 1 | 2.0 |
| Tris (pH 7.5) | 50 | 1.0 |
| MgCl₂ | 10 | 0.2 |
| KCl | 50 | 0.5 |
| Water | | 6.9 |
| Final reaction | | 20 |
| volume (μL) | | |

Results from reactions:

TABLE 20

| | | Conversion |
|---|---|---|
| | SED ID | (%) to |
| Gene name | NO: | product* |
| Wild-type Thermococcus kodakarens | 44 | 98.48 |
| polymerase - ‘Pol6’ | | |
| Wild-type Enterobacteria phage | 42 | 91.26 |
| T4 polymerase - ‘Pol4’ | | |
| Wild-type Thermococcus gorgonarius | 86 | 94.55 |
| polymerase - ‘Tgo’ | | |

*Conversion (%) to product - % area of SEQ ID NO: 2 + SEQ ID NO: 4 + SEQ ID NO: 7 to 38 peaks vs. SEQ ID NO: 38 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform specific modified base filling between two flanking oligonucleotides. Filling the gap by incorporating 5-methylcytosine modification at desired base. Also, that a ligase is able to perform tandem ligation reaction to afford full-length oligonucleotide from the synthesized fragment. Top example polymerase and ligase with product formation greater than 98% by area.

### Example 11: 6 bp 2′F Fully Modified Oligonucleotide Synthesis from 10 bp 2′F Modified Oligonucleotide Primer Gap Filling Between a 26 bp 2′H Oligonucleotide 3′Block on a 45-Bp 2′H Oligonucleotide Template

Aim: Demonstrate gap filling between modified and unmodified oligonucleotide segments with modified deoxyribonucleoside triphosphates to show application of the technology to sugar modification commonly appearing in oligonucleotide therapeutics.

Oligonucleotides (SEQ ID NOs: 1 to 38 & 46 to 54) were acquired from commercially available sources. Primer extending polymerases (SEQ ID NO: 80, 84 and 89) were acquired from commercially available sources and used directly.

Reactions were set up as per Table 21. Oligonucleotides contained within the reactions were annealed by heating to 95° C. and cooling to 15° C. at 0.1° C./sec. Polymerase was then added to commence the reaction. Reactions were incubated at 25° C. for 4 hours. Reactions were then quenched by addition of 60 μL 100 mM EDTA, reactions were then buffer exchanged into water using SEC. Reactions were then analysed by HPLC for the presence of SEQ ID NO: 12.

TABLE 21

| | Final | Volume |
|---|---|---|
| | concentration | per reaction |
| | in reaction (mM) | (μL) |
| Polymerase - SEQ ID NO: | | 1.0 |
| 80, 84 and 89 | | |
| RNAse inhibitor | | 1.0 |
| Template XXL: (2′H) - | 0.04 | 0.8 |
| SEQ ID NO 46 | | |
| 5′primer (2′F) - | 0.04 | 0.8 |
| SEQ ID NO 49 | | |
| 3′block 5 (2′H) - | 0.04 | 0.8 |
| SEQ ID NO 54 | | |
| 2′-F-dATP | 0.5 | 1.0 |
| 2′-F-dGTP | 0.5 | 1.0 |
| 2′-F-dCTP | 0.5 | 1.0 |
| 2′-F-dUTP | 0.5 | 1.0 |
| Tris (pH 7.5) | 50 | 1.0 |
| MgCl₂ | 10 | 0.2 |
| KCl | 50 | 0.5 |
| Water | | 9.9 |
| Final reaction volume (μL) | | 20 |

Results from reactions:

TABLE 22

| | | Conversion |
|---|---|---|
| | SED ID | (%) to |
| Gene name | NO: | product* |
| Mutant Thermus aquaticus | 80 | 10.59 |
| polymerase - ‘KT-V4’ | | |
| Mutant Thermus aquaticus | 84 | 14.83 |
| polymerase - ‘KT-V5’ | | |
| Mutant Thermus aquaticus | 89 | 12.68 |
| polymerase - ‘KT-V6’ | | |

*Conversion (%) to product - % area of SEQ ID NO: 49 + SEQ ID NOs: 7 to 38 peaks vs. SEQ ID NO: 12 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform the base filling between two flanking oligonucleotides extending from 2′-Fluoro modified oligonucleotide primer. Filling the gap by incorporating 2′-Fluoro sugar modifications at desired base positions. Top example polymerase with product formation greater than 14% by area.

### Example 12: 8 bp 2′OH Oligonucleotide Synthesis from 16 bp 2′OH Oligonucleotide Primer Gap Filling Between a 26 bp 2′H Oligonucleotide 3′Block on a 53-Bp 2′H Oligonucleotide Template

Aim: Demonstrate gap filling between an RNA oligonucleotide segment (primer) and unmodified DNA oligonucleotide segment (block) using nucleoside triphosphates to show application of the technology to a sugar group (2′OH) commonly appearing in oligonucleotide therapeutics.

Oligonucleotides (SEQ ID NOs: 1 to 38 & 46 to 54) were acquired from commercially available sources. Primer-extending polymerases (SEQ ID NO: 95) were acquired from commercially available sources and used directly.

Reactions were set up as per Table 23. Oligonucleotides contained within the reactions were annealed by heating to 95° C. and cooling to 15° C. at 0.1° C./sec. Polymerase was then added to commence the reaction. Reactions were incubated at 55° C. for 20 hours. Reactions were then quenched by addition of 60 μL 100 mM EDTA, reactions were then buffer exchanged into water using SEC. Reactions were then analysed by HPLC for the presence of SEQ ID NO: 20.

TABLE 23

| | Final | Volume |
|---|---|---|
| | concentration | per reaction |
| | in reaction (mM) | (μL) |
| Polymerase - SEQ ID NO: 95 | | 1.0 |
| RNAse inhibitor | | 1.0 |
| Template NoRXL: (2′H) - | 0.04 | 0.8 |
| SEQ ID NO: 47 | | |
| 5′primer N + 6 (2′OH) - | 0.04 | 0.8 |
| SEQ ID NO: 51 | | |
| 3′block 2 (2′H) - | 0.04 | 0.8 |
| SEQ ID NO: 4 | | |
| ATP | 0.5 | 1.0 |
| CTP | 0.5 | 1.0 |
| GTP | 0.5 | 1.0 |
| UTP | 0.5 | 1.0 |
| Tris (pH 7.5) | 50 | 1.0 |
| MgCl₂ | 10 | 0.2 |
| KCl | 50 | 0.5 |
| Water | | 9.9 |
| Final reaction | | 20 |
| volume (μL) | | |

Results from reactions:

TABLE 24

| | | Conversion |
|---|---|---|
| | SED ID | (%) to |
| Gene name | NO: | product* |
| Mutant Thermococcus gorgonarius | 95 | 27.10 |
| polymerase - ‘Tgo-V8’ | | |

*Conversion (%) to product - % area of SEQ ID NO: 51 + SEQ ID NOs: 11 to 38peaks vs. SEQ ID NO: 20 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform gap filling between two flanking oligonucleotides extending from an RNA 5′primer. The polymerase gave product formation of 27% by area.

### Example 13: 8 bp 2′OH Oligonucleotide Synthesis from 16 bp 2′OMe Modified Oligonucleotide Primer Gap Filling Between a 26 bp 2′H Oligonucleotide 3′Block on a 53-Bp 2′H Oligonucleotide Template

Aim: Demonstrate gap filling between a modified oligonucleotide segment (primer) and unmodified DNA oligonucleotide segment (block) using nucleoside triphosphates to show application of the technology to a sugar group (2′OMe) commonly appearing in oligonucleotide therapeutics.

Oligonucleotides (SEQ ID NOs: 1 to 38 & 46 to 54) were acquired from commercially available sources. Primer-extending polymerases (SEQ ID NO: 60) were acquired from commercially available sources and used directly.

Reactions were set up as per Table 25. Oligonucleotides contained within the reactions were annealed by heating to 95° C. and cooling to 15° C. at 0.1° C./sec. Polymerase was then added to commence the reaction. Reactions were incubated at 55° C. for 20 hours. Reactions were then quenched by addition of 60 μL 100 mM EDTA, reactions were then buffer exchanged into water using SEC. Reactions were then analysed by HPLC for the presence of SEQ ID NO: 20.

TABLE 25

| | Final | Volume |
|---|---|---|
| | concentration | per reaction |
| | in reaction (mM) | (μL) |
| Polymerase - SEQ ID NO: 60 | | 1.0 |
| RNAse inhibitor | | 1.0 |
| Template NoRXL: (2′H) - | 0.04 | 0.8 |
| SEQ ID NO: 47 | | |
| 5′primer N + 6 | 0.04 | 0.8 |
| (2′OMe + r) - | | |
| SEQ ID NO: 53 | | |
| 3′block 2 (2′H) - | 0.04 | 0.8 |
| SEQ ID NO: 4 | | |
| ATP | 0.5 | 1.0 |
| CTP | 0.5 | 1.0 |
| GTP | 0.5 | 1.0 |
| UTP | 0.5 | 1.0 |
| Tris (pH 7.5) | 50 | 1.0 |
| MgCl₂ | 10 | 0.2 |
| KCl | 50 | 0.5 |
| Water | | 9.9 |
| Final reaction volume (μL) | | 20 |

Results from reactions:

TABLE 26

| | | Conversion |
|---|---|---|
| | SED | (%) to |
| Gene name | ID NO: | product* |
| Mutant Thermococcus | 60 | 32.75 |
| sp. polymerase - ‘9n-V5’ | | |

*Conversion (%) to product - % area of SEQ ID NO: 53 + SEQ ID NOS: 11 to 38peaks vs. SEQ ID NO: 20 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform gap filling between two flanking oligonucleotides extending from a sugar modified 5′primer. The polymerase with product formation of 32% by area.

### Example 14: 54 bp 2′OH Oligonucleotide Synthesis from 16 bp 2′OH Oligonucleotide Primer Gap Filling Between a 26 bp 2′H Oligonucleotide 3′Block on a 100-Bp 2′H Oligonucleotide Template

Aim: Demonstrate longer gap filling between an RNA oligonucleotide segment (primer) and unmodified DNA oligonucleotide segment (block) using a 100-bp 2′H oligonucleotide template and nucleoside triphosphates to show general applicability of the technology.

Oligonucleotides (SEQ ID NOs: 1 to 38 & 46 to 54) were acquired from commercially available sources. Primer-extending polymerases (SEQ ID NO: 56, 60, 84, 89 and 93) were acquired from commercially available sources and used directly.

Reactions were set up as per Table 27. Oligonucleotides contained within the reactions were annealed by heating to 95° C. and cooling to 15° C. at 0.1° C./sec. Polymerase was then added to commence the reaction. Reactions were incubated at 55° C. for 20 hours. Reactions were then quenched by addition of 60 μL 100 mM EDTA, reactions were then buffer exchanged into water using SEC. Reactions were then analysed by HPLC for the presence of SEQ ID NO: 55.

TABLE 27

| | Final | Volume |
|---|---|---|
| | concentration | per reaction |
| | in reaction (mM) | (μL) |
| Polymerase - SEQ ID NO: 56, | | 1.0 |
| 60, 84, 89 and 93 | | |
| RNAse inhibitor | | 1.0 |
| Template 100mer: (2′H) - | 0.04 | 0.8 |
| SEQ ID NO: 48 | | |
| 5′primer N + 6 (2′OH) - | 0.04 | 0.8 |
| SEQ ID NO: 51 | | |
| 3′block 2 (2′H) - | 0.04 | 0.8 |
| SEQ ID NO: 4 | | |
| ATP | 0.5 | 1.0 |
| CTP | 0.5 | 1.0 |
| GTP | 0.5 | 1.0 |
| UTP | 0.5 | 1.0 |
| Tris (pH 7.5) | 50 | 1.0 |
| MgCl₂ | 10 | 0.2 |
| KCl | 50 | 0.5 |
| Water | | 9.9 |
| Final reaction volume (μL) | | 20 |

Results from reactions:

TABLE 28

| | | Conversion |
|---|---|---|
| | SED ID | (%) to |
| Gene name | NO: | product* |
| Wild-type Thermococcus | 56 | 41.07 |
| sp. polymerase - ‘9n’ | | |
| Mutant Thermococcus | 60 | 39.81 |
| sp. polymerase - ‘9n-V5’ | | |
| Mutant Thermus aquaticus | 84 | 57.59 |
| polymerase - ‘KT-V5’ | | |
| Mutant Thermus aquaticus | 89 | 57.58 |
| polymerase - ‘KT-V6’ | | |
| Mutant Thermus aquaticus | 93 | 43.54 |
| polymerase - ‘KT-V1’ | | |

*Conversion (%) to product - % area of SEQ ID NO: 51 + SEQ ID NOS: 11 to 38 peaks vs. SEQ ID NO: 55 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 60-85% buffer B was run over 15 minutes before being stepped down to 60% buffer B for 5 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform longer base filling between two flanking oligonucleotides extending from an RNA 5′primer. Top example polymerase with product formation greater than 57% by area.

### Example 15: 54 bp 2′OH Oligonucleotide Synthesis from 16 bp 2′OMe Oligonucleotide Primer Gap Filling Between a 26 bp 2′H Oligonucleotide 3′Block on a 100-Bp 2′H Oligonucleotide Template

Aim: Demonstrate longer gap filling between a modified oligonucleotide segment (primer) and unmodified DNA oligonucleotide segment (block) using a 100-bp 2′H oligonucleotide template and nucleoside triphosphates to show general applicability of the technology.

Oligonucleotides (SEQ ID NOs: 1 to 38 &46 to 54) were acquired from commercially available sources. Primer-extending polymerases (SEQ ID NO: 60, 83, 84 and 89) were acquired from commercially available sources and used directly.

Reactions were set up as per Table 12. Oligonucleotides contained within the reactions were annealed by heating to 95° C. and cooling to 15° C. at 0.1° C./sec. Polymerase was then added to commence the reaction. Reactions were incubated at 55° C. for 20 hours. Reactions were then quenched by addition of 60 μL 100 mM EDTA, reactions were then buffer exchanged into water using SEC. Reactions were then analysed by HPLC for the presence of SEQ ID NOs: 55.

TABLE 29

| | Final | Volume |
|---|---|---|
| | concentration | per reaction |
| | in reaction (mM) | (μL) |
| Polymerase - SEQ ID NO: 60, | | 1.0 |
| 83, 84 and 89 | | |
| RNAse inhibitor | | 1.0 |
| Template 100mer: (2′H) - | 0.04 | 0.8 |
| SEQ ID NO: 48 | | |
| 5′primer N + 6 | 0.04 | 0.8 |
| (2′OMe + r) - | | |
| SEQ ID NO: 53 | | |
| 3′block 2 (2′H) - | 0.04 | 0.8 |
| SEQ ID NO: 4 | | |
| ATP | 0.5 | 1.0 |
| CTP | 0.5 | 1.0 |
| GTP | 0.5 | 1.0 |
| UTP | 0.5 | 1.0 |
| Tris (pH 7.5) | 50 | 1.0 |
| MgCl₂ | 10 | 0.2 |
| KCl | 50 | 0.5 |
| Water | | 9.9 |
| Final reaction | | 20 |
| volume (μL) | | |

Results from reactions:

TABLE 30

| | | Conversion |
|---|---|---|
| | SED ID | (%) to |
| Gene name | NO: | product* |
| Mutant Thermococcus | 60 | 26.80 |
| sp. polymerase - ‘9n-V5’ | | |
| Mutant Thermococcus gorgonarius | 83 | 20.20 |
| polymerase - ‘Tgo-V9’ | | |
| Mutant Thermus aquaticus | 84 | 27.18 |
| polymerase - ‘KT-V5’ | | |
| Mutant Thermus aquaticus | 89 | 33.12 |
| polymerase - ‘KT-V6’ | | |

*Conversion (%) to product - % area of SEQ ID NO: 53 + SEQ ID NOs: 11 to 38 peaks vs. SEQ ID NO: 55 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 60-85% buffer B was run over 15 minutes before being stepped down to 60% buffer B for 5 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform longer base filling between two flanking oligonucleotides extending from a sugar modified 5′primer. Top example polymerase with product formation greater than 33% by area.

### Example 16: One-Pot 6 bp 2′H Fully PS Modified Oligonucleotide Synthesis from 9 bp 2′MOE Oligonucleotide Primer Gap Filling Between a 7 bp 2′MOE Oligonucleotide 3′Block 2 on a 22-Bp 2′H Oligonucleotide Template and Ligation to Synthesis 22-Bp Product

Aim: Demonstrate gap filling between 2′MOE modified oligonucleotide segments with PS modified deoxyribonucleotides to show application of the technology to backbone modification and sugar modification commonly appearing in gapmer oligonucleotide therapeutics.

Oligonucleotides (SEQ ID NOs: 96 to 98) were acquired from commercially available sources. Primer-extending polymerases (SEQ ID NO: 41) were acquired from commercially available sources and used directly. Double-stranded ligase (SEQ ID NO: 45) was acquired from a commercially available source and used directly.

Reactions were set up as per Table 31. Oligonucleotides contained within the reactions were annealed by heating to 95° C. and cooling to 15° C. at 0.1° C./sec. Polymerase and ligase were then added to commence the reaction. Reactions were incubated at 25° C. for 4 hours. Reactions were then quenched by addition of 60 μL 100 mM EDTA, reactions were then buffer exchanged into water using SEC. Reactions were then analysed by HPLC for the presence of SEQ ID NO: 99.

TABLE 31

| | Final | Volume |
|---|---|---|
| | concentration | per reaction |
| | in reaction (mM) | (μL) |
| Polymerase - SEQ ID NO: 41 | | 1.0 |
| Ligase - SEQ ID NO: 45 | | 1.0 |
| Template H: (2′H) - | 0.04 | 0.8 |
| SEQ ID NO: 96 | | |
| Shortmer 1 (2′MOE) - | 0.04 | 0.8 |
| SEQ ID NO: 97 | | |
| Shortmer 3 (2′MOE) - | 0.04 | 0.8 |
| SEQ ID NO: 98 | | |
| dATPαS | 0.5 | 1.0 |
| dCTPαS | 0.5 | 1.0 |
| dGTPαS | 0.5 | 1.0 |
| dTTPαS | 0.5 | 1.0 |
| ATP | 1 | 2.0 |
| Tris (pH 7.5) | 50 | 1.0 |
| MgCl₂ | 10 | 0.2 |
| Water | | 8.4 |
| Final reaction volume (μL) | | 20 |

Results from reactions:

TABLE 32

| | | Conversion |
|---|---|---|
| | SED ID | (%) to |
| Gene name | NO: | product* |
| Mutant Thermococcus sp. | 41 | 13.2 |
| (strain 9oN-7) polymerase - ‘Pol3’ | | |

*Conversion (%) to product - % area of SEQ ID NO: 97 + SEQ ID NO: 98 + SEQ ID NO: 100 vs. SEQ ID NO: 99 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform specific backbone modified base filling between two flanking 2′MOE oligonucleotides. Filling the gap by incorporating PS modifications between desired bases. Also, that a ligase is able to perform tandem ligation reaction to afford full-length oligonucleotide from the synthesized fragment. Example polymerase and ligase with product formation greater than 13.2% by area.

### Example 17: One-Pot 6 bp 2′H Oligonucleotide Synthesis from 9 bp 2′MOE Oligonucleotide Primer Gap Filling Between a 7 bp 2′MOE Oligonucleotide 3′Block 2′H on a 22-Bp 2′H Oligonucleotide Template and Ligation to Synthesis 22-Bp Product

Aim: Demonstrate gap filling between 2′MOE modified oligonucleotide segments with deoxyribonucleoside triphosphates to show application of the technology to sugar modification commonly appearing in gapmer oligonucleotide therapeutics.

Oligonucleotides (SEQ ID NOs: 96 to 98) were acquired from commercially available sources. Primer-extending polymerases (SEQ ID NO: 40 to 42) were acquired from commercially available sources and used directly. Double-stranded ligase (SEQ ID NO: 45) was acquired from a commercially available source and used directly.

Reactions were set up as per Table 33. Oligonucleotides contained within the reactions were annealed by heating to 95° C. and cooling to 15° C. at 0.1° C./sec. Polymerase and ligase were then added to commence the reaction. Reactions were incubated at 25° C. for 4 hours. Reactions were then quenched by addition of 60 μL 100 mM EDTA, reactions were then buffer exchanged into water using SEC. Reactions were then analysed by HPLC for the presence of SEQ ID NOs: 99.

TABLE 33

| | Final | Volume |
|---|---|---|
| | concentration | per reaction |
| | in reaction (mM) | (μL) |
| Polymerase - SEQ ID NO: 40 to 42 | | 1.0 |
| Ligase - SEQ ID NO: 45 | | 1.0 |
| Template H: (2′H) - | 0.04 | 0.8 |
| SEQ ID NO: 96 | | |
| Shortmer 1 (2′MOE) - | 0.04 | 0.8 |
| SEQ ID NO: 97 | | |
| Shortmer 3 (2′MOE) - | 0.04 | 0.8 |
| SEQ ID NO: 98 | | |
| dATP | 0.5 | 1.0 |
| dCTP | 0.5 | 1.0 |
| dGTP | 0.5 | 1.0 |
| dTTP | 0.5 | 1.0 |
| ATP | 1 | 2.0 |
| Tris (pH 7.5) | 50 | 1.0 |
| MgCl₂ | 10 | 0.2 |
| Water | | 8.4 |
| Final reaction | | 20 |
| volume (μL) | | |

Results from reactions:

TABLE 34

| | SED | Conversion |
|---|---|---|
| | ID | (%) to |
| Gene name | NO: | product* |
| Wild-type Sulfolobus solfataricus polymerase - ‘Pol2’ | 40 | 30.1 |
| Mutant Thermococcus sp. (strain 9oN-7) polymerase - ‘Pol3’ | 41 | 64.0 |
| Wild-type Enterobacteria phage T4 polymerase - ‘Pol4’ | 42 | 52.2 |

*Conversion (%) to product - % area of SEQ ID NO: 97 + SEQ ID NO: 98 + SEQ ID NO: 100 vs. SEQ ID NO: 99 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform specific base filling between two flanking 2′MOE oligonucleotides. Also, that a ligase is able to perform tandem ligation reaction to afford full-length oligonucleotide from the synthesized fragment. Top example polymerase and ligase with product formation greater than 64% by area.

### Example 18: 8 bp 2′OMe Oligonucleotide Synthesis from 16 bp 2′OMe Oligonucleotide Primer Gap Filling Between a 26 bp 2′H Oligonucleotide 3′Block on a 53-Bp 2′H Oligonucleotide Template

Aim: Demonstrate gap filling with 2′OMe sugar modified nucleoside triphosphates between a 2′OMe modified oligonucleotide segment (primer) and unmodified DNA oligonucleotide segment (block) using a 53-bp 2′H oligonucleotide template to show general applicability of the technology.

Oligonucleotides (SEQ ID NOs: 4, 47 and 52) were acquired from commercially available sources. Primer-extending polymerases (SEQ ID NO: 62 and 87) were acquired from commercially available sources and used directly.

Reactions were set up as per Table 35. Oligonucleotides contained within the reactions were annealed by heating to 95° C. and cooling to 15° C. at 0.1° C./sec. Polymerase was then added to commence the reaction. Reactions were incubated at 55° C. for 20 hours. Reactions were then quenched by addition of 60 μL 100 mM EDTA, reactions were then buffer exchanged into water using SEC. Reactions were then analysed by HPLC for the presence of SEQ ID NO: 20.

TABLE 35

| | Final concentration | Volume per reaction |
|---|---|---|
| | in reaction (mM) | (μL) |
| Polymerase - SEQ ID NO: 62 and 87 | | 1.0 |
| Template NoRXL: (2′H) - SEQ ID NO: | 0.04 | 0.8 |
| 47 | | |
| 5′primer N + 6 (2′OMe) - SEQ ID NO: 52 | 0.04 | 0.8 |
| 3′block 2 (2′H) - SEQ ID NO: 4 | 0.04 | 0.8 |
| 2′OMe ATP | 0.5 | 1.0 |
| 2′OMe CTP | 0.5 | 1.0 |
| 2′OMe GTP | 0.5 | 1.0 |
| 2′OMe UTP | 0.5 | 1.0 |
| Tris (pH 7.5) | 50 | 1.0 |
| MgCl₂ | 10 | 0.2 |
| KCl | 50 | 0.5 |
| Water | | 10.9 |
| Final reaction volume (μL) | | 20 |

Results from reactions:

TABLE 36

| | SED | Conversion |
|---|---|---|
| | ID | (%) to |
| Gene name | NO: | product* |
| Mutant Thermococcus kodakarensis polymerase - ‘Kod-V1’ | 62 | 12.0 |
| Mutant Thermococcus gorgonarius polymerase - ‘Tgo-V12’ | 87 | 15.8 |

*Conversion (%) to product - % area of SEQ ID NO: 52 + SEQ ID NOs: 11 to 38 peaks vs. SEQ ID NO: 20 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 60-85% buffer B was run over 15 minutes before being stepped down to 60% buffer B for 5 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform base filling between two flanking oligonucleotides extending from a 2′OMe sugar modified 5′primer with 2′OMe sugar modified nucleoside triphosphates. Top example polymerase with product formation greater than 15% by area.

### Example 19: 850 bp 2′OH Oligonucleotide Synthesis from 16 bp 2′OH Oligonucleotide Primer Gap Filling Between a 26 bp 2′OH Oligonucleotide 3′Block on a 895-Bp 2′H Oligonucleotide Template and Ligation to Produce 892 bp Product

Aim: Demonstrate gap filling between an RNA oligonucleotide segment (primer) and RNA oligonucleotide segment (block) using nucleoside triphosphates to show application of the technology to a sugar group (2′OH) commonly appearing in longer oligonucleotide therapeutics.

Oligonucleotides (SEQ ID NOs: 51, 76 & 101) were acquired from commercially available sources. Primer-extending polymerase (SEQ ID NO: 95) was acquired from commercially available sources and used directly. Double-stranded ligase (SEQ ID NO: 45) was acquired from a commercially available source and used directly.

Reactions were set up as per Table 37. Oligonucleotides contained within the reactions were annealed by heating to 95° C. and cooling to 15° C. at 0.1° C./sec. RNAse inhibitor was then added. Following on, polymerase and ligase were then added to commence the reaction. Reactions were incubated at 25° C. for 16 hours. After which 1 μL of DNase 1 was added and reactions were further incubated at 25° C. for 2 hours. Reactions were then quenched by addition of 4 μL 10 mM EDTA, Reactions were then analysed by Gel electrophoresis, for the presence of SEQ ID NO: 102. Reactions were then analysed by HPLC for the presence of SEQ ID NO: 101.

TABLE 37

| | Final concentration | Volume per reaction |
|---|---|---|
| | in reaction (mM) | (μL) |
| Polymerase - SEQ ID NO: 95 | | 0.25 |
| Ligase - SEQ ID NO: 45 | | 0.12 |
| RNAse inhibitor | | 0.12 |
| Template-900mer: (2′H) - SEQ ID NO: 76 | 0.001 | 1.25 |
| 5′primer N + 6 (2′OH) - SEQ ID NO: 51 | 0.001 | 0.05 |
| 3′block 6 (2′OH) - SEQ ID NO: 101 | 0.001 | 0.05 |
| ATP | 0.2 | 0.0125 |
| CTP | 0.2 | 0.0125 |
| GTP | 0.2 | 0.0125 |
| UTP | 0.2 | 0.0125 |
| Tris (pH 7.5) | 50 | 0.12 |
| MgCl₂ | 10 | 0.25 |
| KCl | 50 | 0.06 |
| Water | | 0.16 |
| Final reaction volume (μL) | | 2.5 |

Results from reactions:

TABLE 38

| | SED | Conversion |
|---|---|---|
| | ID | (%) to |
| Gene name | NO: | product* |
| Mutant Thermococcus gorgonarius polymerase - ‘Tgo-V8’ | 95 | 18.61 |

*Conversion (%) to product - % area of reaction SEQ ID NO: 101 peak vs. negative control SEQ ID NO: 101 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TbuAA, pH 7 in 90% H₂O/10% Acetonitrile
Buffer B: 5 mM TbuAA, pH 7 in 20% H₂O/80% Acetonitrile

Conclusion: Proof of concept that primer-extending polymerases can be used to perform gap filling between two flanking oligonucleotides extending from an RNA 5′primer. The polymerase and tandem ligation gave product formation of 18.6% by area.

### Example 20: 850 bp 2′OH Oligonucleotide Synthesis from 16 bp 2′Ome Oligonucleotide Primer Gap Filling Between a 26 bp 2′OH Oligonucleotide 3′Block on a 895-Bp 2′H Oligonucleotide Template and Ligation to Produce 892 bp Product

Aim: Demonstrate gap filling between an 2′Ome containing oligonucleotide segment (primer) and RNA oligonucleotide segment (block) using nucleoside triphosphates to show application of the technology to a sugar group (2′OH) commonly appearing in longer oligonucleotide therapeutics.

Oligonucleotides (SEQ ID Nos: 53, 76 & 101) were acquired from commercially available sources. Primer-extending polymerase (SEQ ID NO: 95) was acquired from commercially available sources and used directly. Double-stranded ligase (SEQ ID NO: 45) was acquired from a commercially available source and used directly.

Reactions were set up as per Table 39. Oligonucleotides contained within the reactions were annealed by heating to 95° C. and cooling to 15° C. at 0.1° C./sec. RNAse inhibitor was then added. Following on, polymerase and ligase were then added to commence the reaction. Reactions were incubated at 25° C. for 16 hours. After which 1 μL of Dnase 1 was added and reactions were further incubated at 25° C. for 2 hours. Reactions were then quenched by addition of 4 μL 10 mM EDTA, Reactions were then analysed by Gel electrophoresis, for the presence of SEQ ID NO: 103. Reactions were then analysed by HPLC for the presence of SEQ ID NO: 101.

TABLE 39

| | Final concentration | Volume per reaction |
|---|---|---|
| | in reaction (mM) | (μL) |
| Polymerase - SEQ ID NO: 95 | | 0.25 |
| Ligase - SEQ ID NO: 45 | | 0.12 |
| RNAse inhibitor | | 0.12 |
| Template-900mer: (2′H) - SEQ ID NO: 76 | 0.001 | 1.25 |
| 5′primer N + 6 (2′Ome + r) - SEQ ID NO: | 0.001 | 0.05 |
| 53 | | |
| 3′block 6 (2′OH) - SEQ ID NO: 101 | 0.001 | 0.05 |
| ATP | 0.2 | 0.0125 |
| CTP | 0.2 | 0.0125 |
| GTP | 0.2 | 0.0125 |
| UTP | 0.2 | 0.0125 |
| Tris (pH 7.5) | 50 | 0.12 |
| MgCl₂ | 10 | 0.25 |
| KCl | 50 | 0.06 |
| Water | | 0.16 |
| Final reaction volume (μL) | | 2.5 |

Results from reactions:

TABLE 40

| | SED | Conversion |
|---|---|---|
| | ID | (%) to |
| Gene name | NO: | product* |
| Mutant Thermococcus gorgonarius polymerase - ‘Tgo-V8’ | 95 | 36.52 |

*Conversion (%) to product - % area of reaction SEQ ID NO: 101 peak vs. negative control SEQ ID NO: 101 peak at wavelength 260 nm

HPLC analysis was carried out using a Waters Xbridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TbuAA, pH 7 in 90% H₂O/10% Acetonitrile
Buffer B: 5 mM TbuAA, pH 7 in 20% H₂O/80% Acetonitrile

Conclusion: Proof of concept that primer-extending polymerases can be used to perform gap filling between two flanking oligonucleotides extending from an 2′Ome containing 5′primer. The polymerase and tandem ligation gave product formation of 36.52% by area.

### Example 21: 850 bp 2′OH Oligonucleotide Synthesis from 16 bp 2′OH PS Oligonucleotide Primer Gap Filling Between a 26 bp 2′OH Oligonucleotide 3′Block on a 895-Bp 2′H Oligonucleotide Template and Ligation to Produce 892 bp Product

Aim: Demonstrate gap filling between an RNA phosphorothioate containing oligonucleotide segment (primer) and RNA oligonucleotide segment (block) using nucleoside triphosphates to show application of the technology to a sugar group (2′OH) commonly appearing in longer oligonucleotide therapeutics.

Oligonucleotides (SEQ ID Nos: 76, 101 & 104) were acquired from commercially available sources. Primer-extending polymerase (SEQ ID NO: 95) was acquired from commercially available sources and used directly. Double-stranded ligase (SEQ ID NO: 45) was acquired from a commercially available source and used directly.

Reactions were set up as per Table 41. Oligonucleotides contained within the reactions were annealed by heating to 95° C. and cooling to 15° C. at 0.1° C./sec. RNAse inhibitor was then added. Following on, polymerase and ligase were then added to commence the reaction. Reactions were incubated at 25° C. for 16 hours. After which 1 μL of Dnase 1 was added and reactions were further incubated at 25° C. for 2 hours. Reactions were then quenched by addition of 4 μL 10 mM EDTA, Reactions were then analysed by Gel electrophoresis, for the presence of SEQ ID NO: 105. Reactions were then analysed by HPLC for the presence of SEQ ID NO: 101.

TABLE 41

| | Final concentration | Volume per reaction |
|---|---|---|
| | in reaction (mM) | (μL) |
| Polymerase - SEQ ID NO: 95 | | 0.25 |
| Ligase - SEQ ID NO: 45 | | 0.12 |
| RNAse inhibitor | | 0.12 |
| Template-900mer: (2′H) - SEQ ID NO: 76 | 0.001 | 1.25 |
| 5′primer N + 6_2′OH_PS (2′OH)- SEQ ID | 0.001 | 0.05 |
| NO: 104 | | |
| 3′block 6 (2′OH) - SEQ ID NO: 101 | 0.001 | 0.05 |
| ATP | 0.2 | 0.0125 |
| CTP | 0.2 | 0.0125 |
| GTP | 0.2 | 0.0125 |
| UTP | 0.2 | 0.0125 |
| Tris (pH 7.5) | 50 | 0.12 |
| MgCl₂ | 10 | 0.25 |
| KCl | 50 | 0.06 |
| Water | | 0.16 |
| Final reaction volume (μL) | | 2.5 |

Results from reactions:

TABLE 42

| | SED | Conversion |
|---|---|---|
| | ID | (%) to |
| Gene name | NO: | product* |
| Mutant Thermococcus gorgonarius polymerase - ‘Tgo-V8’ | 95 | 10.78 |

*Conversion (%) to product - % area of reaction SEQ ID NO: 101 peak vs. negative control SEQ ID NO: 101 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TbuAA, pH 7 in 90% H₂O/10% Acetonitrile
Buffer B: 5 mM TbuAA, pH 7 in 20% H₂O/80% Acetonitrile

Conclusion: Proof of concept that primer-extending polymerases can be used to perform gap filling between two flanking oligonucleotides extending from an 2′OH phosphorothioate containing 5′primer. The polymerase and tandem ligation gave product formation of 10.78% by area.

### Example 22: 850 bp 2′OH Oligonucleotide Synthesis Containing Pseudouridine from 16 bp 2′OH Oligonucleotide Primer Gap Filling Between a 26 bp 2′OH Oligonucleotide 3′Block on a 895-Bp 2′H Oligonucleotide Template and Ligation to Produce 892 bp Product

Aim: Demonstrate gap filling between an RNA oligonucleotide segment (primer) and RNA oligonucleotide segment (block) using pseudouridine nucleoside triphosphate and nucleoside triphosphates to show application of the technology to a base modification (4)) commonly appearing in oligonucleotide therapeutics.

Oligonucleotides (SEQ ID Nos: 51, 76 & 101) were acquired from commercially available sources. Primer-extending polymerase (SEQ ID NO: 95) was acquired from commercially available sources and used directly. Double-stranded ligase (SEQ ID NO: 45) was acquired from a commercially available source and used directly.

Reactions were set up as per Table 43. Oligonucleotides contained within the reactions were annealed by heating to 95° C. and cooling to 15° C. at 0.1° C./sec. RNAse inhibitor was then added. Following on, polymerase and ligase were then added to commence the reaction. Reactions were incubated at 25° C. for 16 hours. After which 1 μL of Dnase 1 was added and reactions were further incubated at 25° C. for 2 hours. Reactions were then quenched by addition of 4 μL 10 mM EDTA, Reactions were then analysed by Gel electrophoresis, for the presence of SEQ ID NO: 106. Reactions were then analysed by HPLC for the presence of SEQ ID NO: 101.

TABLE 43

| | Final concentration | Volume per reaction |
|---|---|---|
| | in reaction (mM) | (μL) |
| Polymerase - SEQ ID NO: 95 | | 0.25 |
| Ligase - SEQ ID NO: 45 | | 0.12 |
| RNAse inhibitor | | 0.12 |
| Template-900mer: (2′H) - SEQ ID NO: 76 | 0.001 | 1.25 |
| 5′primer N + 6 (2′OH) - SEQ ID NO: 51 | 0.001 | 0.05 |
| 3′block 6 (2′OH) - SEQ ID NO: 101 | 0.001 | 0.05 |
| ATP | 0.2 | 0.0125 |
| CTP | 0.2 | 0.0125 |
| GTP | 0.2 | 0.0125 |
| ΨTP | 0.2 | 0.0125 |
| Tris (pH 7.5) | 50 | 0.12 |
| MgCl₂ | 10 | 0.25 |
| KCl | 50 | 0.06 |
| Water | | 0.16 |
| Final reaction volume (μL) | | 2.5 |

Results from reactions:

TABLE 44

| | SED | Conversion |
|---|---|---|
| | ID | (%) to |
| Gene name | NO: | product* |
| Mutant Thermococcus gorgonarius polymerase - ‘Tgo-V8’ | 95 | 24.4 |

*Conversion (%) to product - % area of reaction SEQ ID NO: 101 peak vs. negative control SEQ ID NO: 101 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform gap filling to form a pseudouridine containing sequence. The polymerase and tandem ligation gave product formation of 24.4% by area.

### Example 23: 850 bp 2′OH Oligonucleotide Synthesis Containing N1-Methylpseudouridine from 16 bp 2′OH Oligonucleotide Primer Gap Filling Between a 26 bp 2′OH Oligonucleotide 3′Block on a 895-Bp 2′H Oligonucleotide Template and Ligation to Produce 892 bp Product

Aim: Demonstrate gap filling between an RNA oligonucleotide segment (primer) and RNA oligonucleotide segment (block) using N1-Methylpseudouridine nucleoside triphosphate and nucleoside triphosphates to show application of the technology to a base modification (m14J) commonly appearing in oligonucleotide therapeutics.

Oligonucleotides (SEQ ID NOs: 51, 76 & 101) were acquired from commercially available sources. Primer-extending polymerase (SEQ ID NO: 95) was acquired from commercially available sources and used directly. Double-stranded ligase (SEQ ID NO: 45) was acquired from a commercially available source and used directly.

Reactions were set up as per Table 45. Oligonucleotides contained within the reactions were annealed by heating to 95° C. and cooling to 15° C. at 0.1° C./sec. RNAse inhibitor was then added. Following on, polymerase and ligase were then added to commence the reaction. Reactions were incubated at 25° C. for 16 hours. After which 1 μL of DNase 1 was added and reactions were further incubated at 25° C. for 2 hours. Reactions were then quenched by addition of 4 μL 10 mM EDTA, Reactions were then analysed by Gel electrophoresis, for the presence of SEQ ID NO: 107. Reactions were then analysed by HPLC for the presence of SEQ ID NO: 101

TABLE 45

| | Final concentration | Volume per reaction |
|---|---|---|
| | in reaction (mM) | (μL) |
| Polymerase - SEQ ID NO: 95 | | 0.25 |
| Ligase - SEQ ID NO: 45 | | 0.12 |
| RNAse inhibitor | | 0.12 |
| Template-900mer: (2′H) - SEQ ID NO: 76 | 0.001 | 1.25 |
| 5′primer N + 6 (2′OH) - SEQ ID NO: 51 | 0.001 | 0.05 |
| 3′block 6 (2′OH) - SEQ ID NO: 101 | 0.001 | 0.05 |
| ATP | 0.2 | 0.0125 |
| CTP | 0.2 | 0.0125 |
| GTP | 0.2 | 0.0125 |
| m1ΨTP | 0.2 | 0.0125 |
| Tris (pH 7.5) | 50 | 0.12 |
| MgCl₂ | 10 | 0.25 |
| KCl | 50 | 0.06 |
| Water | | 0.16 |
| Final reaction volume (μL) | | 2.5 |

Results from reactions:

TABLE 46

| | SED | Conversion |
|---|---|---|
| | ID | (%) to |
| Gene name | NO: | product* |
| Mutant Thermococcus gorgonarius polymerase - ‘Tgo-V8’ | 95 | 21.64 |

*Conversion (%) to product - % area of reaction SEQ ID NO: 101 peak vs. negative control SEQ ID NO: 101 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform gap filling to form a N1-Methylpseudouridine containing sequence. The polymerase and tandem ligation gave product formation of 21.64% by area.

### Example 24: 850 bp 2′OH PS Modified Oligonucleotide Synthesis from 16 bp 2′OH Oligonucleotide Primer Gap Filling Between a 26 bp 2′OH Oligonucleotide 3′Block on a 895-Bp 2′H Oligonucleotide Template and Ligation to Produce 892 bp Product

Aim: Demonstrate gap filling between an RNA oligonucleotide segment (primer) and RNA oligonucleotide segment (block) using modified nucleoside triphosphates to show application of the technology to a backbone modification commonly appearing in oligonucleotide therapeutics.

Oligonucleotides (SEQ ID NOs: 51, 76 & 101) were acquired from commercially available sources. Primer-extending polymerase (SEQ ID NO: 95) was acquired from commercially available sources and used directly. Double-stranded ligase (SEQ ID NO: 45) was acquired from a commercially available source and used directly.

Reactions were set up as per Table 45. Oligonucleotides contained within the reactions were annealed by heating to 95° C. and cooling to 15° C. at 0.1° C./sec. RNAse inhibitor was then added. Following on, polymerase and ligase were then added to commence the reaction. Reactions were incubated at 25° C. for 16 hours. After which 1 μL of DNase 1 was added and reactions were further incubated at 25° C. for 2 hours. Reactions were then quenched by addition of 4 μL 10 mM EDTA, Reactions were then analysed by Gel electrophoresis, for the presence of SEQ ID NO: 108. Reactions were then analysed by HPLC for the presence of SEQ ID NO: 101

TABLE 47

| | Final concentration | Volume per reaction |
|---|---|---|
| | in reaction (mM) | (μL) |
| Polymerase - SEQ ID NO: 95 | | 0.25 |
| Ligase - SEQ ID NO: 45 | | 0.12 |
| RNAse inhibitor | | 0.12 |
| Template-900mer: (2′H) - SEQ ID NO: 76 | 0.001 | 1.25 |
| 5′primer N + 6 (2′OH) - SEQ ID NO: 51 | 0.001 | 0.05 |
| 3′block 6 (2′OH) - SEQ ID NO: 101 | 0.001 | 0.05 |
| ATPαS | 0.2 | 0.0125 |
| CTPαS | 0.2 | 0.0125 |
| GTPαS | 0.2 | 0.0125 |
| UTPαS | 0.2 | 0.0125 |
| Tris (pH 7.5) | 50 | 0.12 |
| MgCl₂ | 10 | 0.25 |
| KCl | 50 | 0.06 |
| Water | | 0.16 |
| Final reaction volume (μL) | | 2.5 |

Results from reactions:

TABLE 48

| | SED | Conversion |
|---|---|---|
| | ID | (%) to |
| Gene name | NO: | product* |
| Mutant Thermococcus gorgonarius polymerase - ‘Tgo-V8’ | 95 | 4.95 |

*Conversion (%) to product - % area of reaction SEQ ID NO: 101 peak vs. negative control SEQ ID NO: 101 peak at wavelength 260 nm

HPLC analysis was carried out using a Waters XBridge Peptide BEH C18 Column (300 Å, 3.5 μm, 2.1 mm×150 mm), running at 0.5 ml/min while absorbance was monitored at 260 nm. The column was maintained at 50° C. 5 μl of sample was injected and a gradient from 40-95% buffer B was run over 25 minutes before being stepped down to 40% buffer B for 4 minutes.
Buffer A: 5 mM TBuAA, pH 7 in 90% H₂O/10% Acetonitrile
Buffer B: 5 mM TBuAA, pH 7 in 20% H₂O/80% Acetonitrile

Conclusion: Proof of concept that primer-extending polymerases can be used to perform gap filling to form a phosphorothioate modified sequence. The polymerase and tandem ligation gave product formation of 21.64% by area.

### OVERALL CONCLUSIONS

The inventors have shown that it is possible to synthesise polynucleotides or oligonucleotides, including polynucleotides or oligonucleotides with a range of therapeutically relevant modifications, by assembling polynucleotide or oligonucleotide segments on a complementary template, extending the segments using a polymerase to fill in the gaps and ligating the segments together and separating the product polynucleotide or oligonucleotide from both impurities and its complementary template in an efficient process that is scalable and suitable for large-scale therapeutic polynucleotide and oligonucleotide manufacture.

In using the inherent properties of nucleic acids e.g. DNA or RNA, to recognise complementary sequences specifically and bind complementary sequences with an affinity that reflects both the fidelity of the complementary sequence and the length of the complementary sequence, the inventors have been able to produce polynucleotides and oligonucleotides of high purity without the need for chromatography, which both improves the efficiency of the production process and the scalability of the process. By recovering the template in an unchanged state during the separation process the inventors are able to reuse the template for further rounds of synthesis and so have avoided the economic consequences of having to make one equivalent of template for every equivalent of product oligonucleotide formed. It is also possible to synthesise oligonucleotides in solution in some embodiments, avoiding the scale-up constraints imposed by solid-phase methods.

Finally, although wild-type polymerases are effective, modifications of the polymerase (resulting in a mutant or engineered polymerase) may be utilised to increase efficiency, template recovery, or incorporate modified nucleotides. Similarly, although wild-type ligases are effective, with appropriate mutation and evolution of ligases, ligation efficiency can be increased and appropriately modified ligases are effective catalysts for synthesizing oligonucleotides which contain multiple modifications.

SEQUENCE LISTING

| SEQ ID | |
|---|---|
| NO: | Sequence Identifier |
| 1 | /5Biosg/GCTAATGGCTTTGGTGCGAAGCAGACTGAGGCACCGAGGAGT - ‘Template’ |
| 2 | ACTCCTCGGT - ‘5′primer N’ |
| 3 | /5phos/AGTCTGCTTCGCACCAAAGCCATTAGC - ‘3′block 1’ |
| 4 | /5phos/GTCTGCTTCGCACCAAAGCCATTAGC - ‘3′block 2’ |
| 5 | /5Phos/GCACCAAAGC/Me-dC/ATTAGC - ‘3′block 3’ |
| 6 | /5Phos/GC/Me-dC/ATTAGC - ‘3′block 4’ |
| 7 | ACTCCTCGGTG - ‘Primer N + 1’ |
| 8 | ACTCCTCGGTGC - ‘Primer N + 2’ |
| 9 | ACTCCTCGGTGCC - ‘Primer N + 3’ |
| 10 | ACTCCTCGGTGCCT - ‘Primer N + 4’ |
| 11 | ACTCCTCGGTGCCTC - ‘Primer N + 5’ |
| 12 | ACTCCTCGGTGCCTCA - ‘Primer N + 6’ |
| 13 | ACTCCTCGGTGCCTCAG - ‘Primer N + 7’ |
| 14 | ACTCCTCGGTGCCTCAGT - ‘Primer N + 8’ |
| 15 | ACTCCTCGGTGCCTCAGTC - ‘Primer N + 9’ |
| 16 | ACTCCTCGGTGCCTCAGTCT - ‘Primer N + 10’ |
| 17 | ACTCCTCGGTGCCTCAGTCTG - ‘Primer N + 11’ |
| 18 | ACTCCTCGGTGCCTCAGTCTGC - ‘Primer N + 12’ |
| 19 | ACTCCTCGGTGCCTCAGTCTGCT - ‘Primer N + 13’ |
| 20 | ACTCCTCGGTGCCTCAGTCTGCTT - ‘Primer N + 14’ |
| 21 | ACTCCTCGGTGCCTCAGTCTGCTTC - ‘Primer N + 15’ |
| 22 | ACTCCTCGGTGCCTCAGTCTGCTTCG - ‘Primer N + 16’ |
| 23 | ACTCCTCGGTGCCTCAGTCTGCTTCGC - ‘Primer N + 17’ |
| 24 | ACTCCTCGGTGCCTCAGTCTGCTTCGCA - ‘Primer N + 18’ |
| 25 | ACTCCTCGGTGCCTCAGTCTGCTTCGCAC - ‘Primer N + 19’ |
| 26 | ACTCCTCGGTGCCTCAGTCTGCTTCGCACC - ‘Primer N + 20’ |
| 27 | ACTCCTCGGTGCCTCAGTCTGCTTCGCACCA - ‘Primer N + 21’ |
| 28 | ACTCCTCGGTGCCTCAGTCTGCTTCGCACCAA - ‘Primer N + 22’ |
| 29 | ACTCCTCGGTGCCTCAGTCTGCTTCGCACCAAA - ‘Primer N + 23’ |
| 30 | ACTCCTCGGTGCCTCAGTCTGCTTCGCACCAAAG - ‘Primer N + 24’ |
| 31 | ACTCCTCGGTGCCTCAGTCTGCTTCGCACCAAAGC - ‘Primer N + 25’ |
| 32 | ACTCCTCGGTGCCTCAGTCTGCTTCGCACCAAAGCC - ‘Primer N + 26’ |
| 33 | ACTCCTCGGTGCCTCAGTCTGCTTCGCACCAAAGCCA - ‘Primer N + 27’ |
| 34 | ACTCCTCGGTGCCTCAGTCTGCTTCGCACCAAAGCCAT - ‘Primer N + 28’ |
| 35 | ACTCCTCGGTGCCTCAGTCTGCTTCGCACCAAAGCCATT - `Primer N + 29’ |
| 36 | ACTCCTCGGTGCCTCAGTCTGCTTCGCACCAAAGCCATTA - ‘Primer N + 30’ |
| 37 | ACTCCTCGGTGCCTCAGTCTGCTTCGCACCAAAGCCATTAG - ‘Primer N + 31’ |
| 38 | ACTCCTCGGTGCCTCAGTCTGCTTCGCACCAAAGCCATTAGC - ‘Primer N + 32’ |
| 39 | Wild-type Escherichia phage T7 polymerase - ‘Pol1’ |
| 40 | Wild-type Sulfolobus solfataricus polymerase - ‘Pol2’ |
| 41 | Mutant Thermococcus sp. (strain 9oN-7) polymerase - ‘Pol3’ |
| 42 | Wild-type Enterobacteria phage T4 polymerase - ‘Pol4’ |
| 43 | Wild-type Thermus aquaticus polymerase - ‘Pol5’ |
| 44 | Wild-type Thermococcus kodakarens polymerase - ‘Pol6’ |
| 45 | Wild-type Enterobacteria phage T3 ligase - ‘Lig1’ |
| 46 | /5Biosg/GCTAATGGCTTTGGTGCGAAGCAGACTGAGGCACCGAGGAGT*T*T*T- |
| | ‘Template XXL’ |
| 47 | CTAATGGCTTTGGTGCGAAGCAGACCTGATGACTGAGGCACCGAGGAGT*T*T*T*T- |
| | ‘Template NoRXL’ |
| 48 | C*T*A*A*T*GGCTTTGGTGCGAAGCAGACCTCTTCACCTTTGCTCACCATTTGTAGTCCAT |
| | CGGATATATCTCCTTCGGATCCTGAGGCACCGAGGAG*T*T*T*T*T - ‘Template- |
| | 100 mer’ |
| 49 | ACTCCfUfCfGfGfU - ‘5′primer N_2‘F’ |
| 50 | ACTCCTCGGTGCCTCA - ‘5′primer N + 6_2‘H’ |
| 51 | rArCrUrCrCrUrCrGrGrUrGrCrCrUrCrA - ‘5′primer N + 6_2′OH’ |
| 52 | mAmCmUmCmCmUmCmGmGmUmGmCmCmUmCmA - ‘5′primer N + 6_2′OMe’ |
| 53 | mAmCmUmCmCmUmCmGmGmUmGmCmCrUrCrA - ‘5′primer N + 6_2′OMe + r’ |
| 54 | GTCTGCTTCGCACCAAAGCCATTAGC - ‘3′block 5’ |
| 55 | ACTCCTCGGTGCCTCAGGATCCGAAGGAGATATATCCGATGGACTACAAATGGTGAGCAAA |
| | GGTGAAGAG - ‘Primer N + 60’ |
| 56 | Wild-type Thermococcus sp. polymerase - ‘9n’ |
| 57 | Mutant Thermococcus gorgonarius polymerase - ‘Tgo-V2’ |
| 58 | Mutant Thermococcus gorgonarius polymerase - ‘Tgo-V16’ |
| 59 | Mutant Thermus aquaticus polymerase - ‘KT-V9’ |
| 60 | Mutant Thermococcus sp. polymerase - ‘9n-V5’ |
| 61 | Mutant Thermococcus sp. polymerase - ‘9n-V1’ |
| 62 | Mutant Thermococcus kodakarensis polymerase - ‘Kod-V1’ |
| 63 | Mutant Thermococcus gorgonarius polymerase - ‘Tgo-V3’ |
| 64 | Wild-type Thermococcus litoralis polymerase - ‘Vent’ |
| 65 | Mutant Thermus aquaticus polymerase - ‘KT-V2’ |
| 66 | Mutant Thermococcus sp. polymerase - ‘9n-V2’ |
| 67 | Mutant Thermococcus kodakarensis polymerase - ‘Kod-V2’ |
| 68 | Mutant Thermococcus gorgonarius polymerase - ‘Tgo-V4’ |
| 69 | Mutant Thermococcus litoralis polymerase - ‘Vent-V1’ |
| 70 | Mutant Thermus aquaticus polymerase - ‘KT-V10’ |
| 71 | Mutant Thermococcus sp. polymerase - ‘9n-V3’ |
| 72 | Mutant Thermococcus kodakarensis polymerase - ‘Kod-V3’ |
| 73 | Mutant Thermococcus gorgonarius polymerase - ‘Tgo-V5’ |
| 74 | Mutant Thermococcus litoralis polymerase - ‘Vent-V2’ |
| 75 | Mutant Thermus aquaticus polymerase - ‘KT-V3’ |
| 76 | CTAATGGCTTTGGTGCGAAGCAGACTTATTGCTCAGCGGTGGCAGCAGCCAACTCAGCTTC |
| | CTTTCGGGCTTTGTTAGCAGCCGGATCTCTCGAGTTACTTATACAGTTCATCCATACCACCG |
| | GTACTATGGCGACCTTCTGCACGTTCATACTGTTCAACAATGGTATAATCCTCGTTGTGGC |
| | TGGTGATATCCAGTTTGATATTAACATTATATGCACCAGGCAGCTGAACCGGCTTTTTGGC |
| | TTTGTAGGTGGTTTTAACTTCTGCATCATAATGACCGCCATCTTTCAGCTTCAGACGCTGTT |
| | TAATTTCACCTTTCAGTGCGCCATCTTCCGGATACATACGTTCGCTGCTTGCTTCCCAACCC |
| | ATGGTCTTTTTCTGCATAACCGGACCATCACTCGGAAAATTCGTGCCACGCAGTTTAACTTT |
| | ATAGATAAATTCACCATCCTGCAGGCTGCTATCCTGTGTAACGGTAACAACACCACCATCTT |
| | CAAAATTCATCACACGTTCCCATTTAAAACCTTCCGGAAAGCTCAGTTTCAGATAATCCGGG |
| | ATATCTGCCGGATGTTTAACATAGGCTTTGCTACCATACATAAACTGCGGACTCAGAATATC |
| | CCATGCAAACGGCAGCGGACCACCTTTGGTAACTTTCAGTTTTGCGGTCTGGGTGCCTTCA |
| | TACGGACGACCTTCGCCTTCACCTTCAATTTCAAATTCGTGGCCATTAACGCTACCTTCCAT |
| | ATGAACTTTGAAGCGCATGAATTCTTTGATGATGGCCATATTATCCTCTTCACCTTTGCTCA |
| | CCATGCTAGCCATATGGCTGCCGCGCGGCACCAGGCCGCTGCTGTGATGATGATGATGAT |
| | GGCTGCTGCCCATTGAGGCACCGAGGAGTTTTT - ‘Template-900 mer’ |
| 77 | Mutant Thermococcus sp. polymerase - ‘9n-V4’ |
| 78 | Mutant Thermococcus kodakarensis polymerase - ‘Kod-V5’ |
| 79 | Mutant Thermococcus gorgonarius polymerase - ‘Tgo-V6’ |
| 80 | Mutant Thermus aquaticus polymerase - ‘KT-V4’ |
| 81 | Mutant Thermococcus sp. polymerase - ‘9n-V7’ |
| 82 | Ψild-type Pyrococcus furiosus polymerase - Pfu |
| 83 | Mutant Thermococcus gorgonarius polymerase - ‘Tgo-V9’ |
| 84 | Mutant Thermus aquaticus polymerase - ‘KT-V5’ |
| 85 | Mutant Thermococcus sp. polymerase - ‘9n-V8’ |
| 86 | Ψild-type Thermococcus gorgonarius polymerase - ‘Tgo’ |
| 87 | Mutant Thermococcus gorgonarius polymerase - ‘Tgo-V12’ |
| 88 | Mutant Thermus aquaticus polymerase - ‘KT-V8’ |
| 89 | Mutant Thermus aquaticus polymerase - ‘KT-V6’ |
| 90 | Mutant Thermococcus sp. polymerase - ‘9n-V9’ |
| 91 | Mutant Thermococcus gorgonarius polymerase - ‘Tgo-V1’ |
| 92 | Mutant Thermococcus gorgonarius polymerase - ‘Tgo-V14’ |
| 93 | Mutant Thermus aquaticus polymerase - ‘KT-V1’ |
| 94 | Mutant Thermus aquaticus polymerase - ‘KT-V7’ |
| 95 | Mutant Thermococcus gorgonarius polymerase - ‘Tgo-V8’ |
| 96 | /5Biosg/GCA CTT CGC TTC ACC TCT CTG C*T*T* T - ‘Template H’ |
| 97 | eGeCeAeGeAeGeAGG - ‘Shortmer 1 (2′MOE)’ |
| 98 | /5phos/GAeAeGeTeGeC - ‘Shortmer 3 (2′MOE)’ |
| 99 | eGeCeAeGeAeGeAGGTGAAGCGAeAeGeTeGeC - ‘Product (2′MOE)’ |
| 100 | eGeCeAeGeAeGeAGGTGAAGC - `Shortmer 1 + 2 (2′MOE)’ |
| 101 | /5Phos/rGrU rCrUrG rCrUrU rCrGrC rArCrC rArArA rGrCrC mA*mU*mU* mA*mG*mC - |
| | ‘3′block 6’ |
| 102 | rArCrUrCrCrUrCrGrGrUrGrCrCrUrCrArArUrGrGrGrCrArGrCrArGrCrCrArUrCrArUrCrArUrC |
| | rArUrCrArUrCrArCrArGrCrArGrCrGrGrCrCrUrGrGrUrGrCrCrGrCrGrCrGrGrCrArGrCrCrArU |
| | rArUrGrGrCrUrArGrCrArUrGrGrUrGrArGrCrArArArGrGrUrGrArArGrArGrGrArUrArArUrAr |
| | UrGrGrCrCrArUrCrArUrCrArArArGrArArUrUrCrArUrGrCrGrCrUrUrCrArArArGrUrUrCrArUr |
| | ArUrGrGrArArGrGrUrArGrCrGrUrUrArArUrGrGrCrCrArCrGrArArUrUrUrGrArArArUrUrGrA |
| | rArGrGrUrGrArArGrGrCrGrArArGrGrUrCrGrUrCrCrGrUrArUrGrArArGrGrCrArCrCrCrArGrA |
| | rCrCrGrCrArArArArCrUrGrArArArGrUrUrArCrCrArArArGrGrUrGrGrUrCrCrGrCrUrGrCrCrG |
| | rUrUrUrGrCrArUrGrGrGrArUrArUrUrCrUrGrArGrUrCrCrGrCrArGrUrUrUrArUrGrUrArUrGr |
| | GrUrArGrCrArArArGrCrCrUrArUrGrUrUrArArArCrArUrCrCrGrGrCrArGrArUrArUrCrCrCrGr |
| | GrArUrUrArUrCrUrGrArArArCrUrGrArGrCrUrUrUrCrCrGrGrArArGrGrUrUrUrUrArArArUrG |
| | rGrGrArArCrGrUrGrUrGrArUrGrArArUrUrUrUrGrArArGrArUrGrGrUrGrGrUrGrUrUrGrUrUr |
| | ArCrCrGrUrUrArCrArCrArGrGrArUrArGrCrArGrCrCrUrGrCrArGrGrArUrGrGrUrGrArArUrUr |
| | UrArUrCrUrArUrArArArGrUrUrArArArCrUrGrCrGrUrGrGrCrArCrGrArArUrUrUrUrCrCrGrAr |
| | GrUrGrArUrGrGrUrCrCrGrGrUrUrArUrGrCrArGrArArArArArGrArCrCrArUrGrGrGrUrUrGrG |
| | rGrArArGrCrArArGrCrArGrCrGrArArCrGrUrArUrGrUrArUrCrCrGrGrArArGrArUrGrGrCrGrC |
| | rArCrUrGrArArArGrGrUrGrArArArUrUrArArArCrArGrCrGrUrCrUrGrArArGrCrUrGrArArArG |
| | rArUrGrGrCrGrGrUrCrArUrUrArUrGrArUrGrCrArGrArArGrUrUrArArArArCrCrArCrCrUrArC |
| | rArArArGrCrCrArArArArArGrCrCrGrGrUrUrCrArGrCrUrGrCrCrUrGrGrUrGrCrArUrArUrArA |
| | rUrGrUrUrArArUrArUrCrArArArCrUrGrGrArUrArUrCrArCrCrArGrCrCrArCrArArCrGrArGrG |
| | rArUrUrArUrArCrCrArUrUrGrUrUrGrArArCrArGrUrArUrGrArArCrGrUrGrCrArGrArArGrGrU |
| | rCrGrCrCrArUrArGrUrArCrCrGrGrUrGrGrUrArUrGrGrArUrGrArArCrUrGrUrArUrArArGrUr |
| | ArArCrUrCrGrArGrArGrArUrCrCrGrGrCrUrGrCrUrArArCrArArArGrCrCrCrGrArArArGrGrAr |
| | ArGrCrUrGrArGrUrUrGrGrCrUrGrCrUrGrCrCrArCrCrGrCrUrGrArGrCrArArUrArArGrUrCrU |
| | rGrCrUrUrCrGrCrArCrCrArArArGrCrCmA*mU*mU*mA*mG*mC - ‘Product-892 mer 1’ |
| 103 | mAmCmUmCmCmUmCmGmGmUmGmCmCrUrCrArArUrGrGrGrCrArGrCrArGrCrCrArUr |
| | CrArUrCrArUrCrArUrCrArUrCrArCrArGrCrArGrCrGrGrCrCrUrGrGrUrGrCrCrGrCrGrCrGrGr |
| | CrArGrCrCrArUrArUrGrGrCrUrArGrCrArUrGrGrUrGrArGrCrArArArGrGrUrGrArArGrArGrG |
| | rArUrArArUrArUrGrGrCrCrArUrCrArUrCrArArArGrArArUrUrCrArUrGrCrGrCrUrUrCrArArA |
| | rGrUrUrCrArUrArUrGrGrArArGrGrUrArGrCrGrUrUrArArUrGrGrCrCrArCrGrArArUrUrUrGr |
| | ArArArUrUrGrArArGrGrUrGrArArGrGrCrGrArArGrGrUrCrGrUrCrCrGrUrArUrGrArArGrGrC |
| | rArCrCrCrArGrArCrCrGrCrArArArArCrUrGrArArArGrUrUrArCrCrArArArGrGrUrGrGrUrCrCr |
| | GrCrUrGrCrCrGrUrUrUrGrCrArUrGrGrGrArUrArUrUrCrUrGrArGrUrCrCrGrCrArGrUrUrUrA |
| | rUrGrUrArUrGrGrUrArGrCrArArArGrCrCrUrArUrGrUrUrArArArCrArUrCrCrGrGrCrArGrArU |
| | rArUrCrCrCrGrGrArUrUrArUrCrUrGrArArArCrUrGrArGrCrUrUrUrCrCrGrGrArArGrGrUrUr |
| | UrUrArArArUrGrGrGrArArCrGrUrGrUrGrArUrGrArArUrUrUrUrGrArArGrArUrGrGrUrGrGrU |
| | rGrUrUrGrUrUrArCrCrGrUrUrArCrArCrArGrGrArUrArGrCrArGrCrCrUrGrCrArGrGrArUrGr |
| | GrUrGrArArUrUrUrArUrCrUrArUrArArArGrUrUrArArArCrUrGrCrGrUrGrGrCrArCrGrArArUr |
| | UrUrUrCrCrGrArGrUrGrArUrGrGrUrCrCrGrGrUrUrArUrGrCrArGrArArArArArGrArCrCrArUr |
| | GrGrGrUrUrGrGrGrArArGrCrArArGrCrArGrCrGrArArCrGrUrArUrGrUrArUrCrCrGrGrArArG |
| | rArUrGrGrCrGrCrArCrUrGrArArArGrGrUrGrArArArUrUrArArArCrArGrCrGrUrCrUrGrArArG |
| | rCrUrGrArArArGrArUrGrGrCrGrGrUrCrArUrUrArUrGrArUrGrCrArGrArArGrUrUrArArArArC |
| | rCrArCrCrUrArCrArArArGrCrCrArArArArArGrCrCrGrGrUrUrCrArGrCrUrGrCrCrUrGrGrUrG |
| | rCrArUrArUrArArUrGrUrUrArArUrArUrCrArArArCrUrGrGrArUrArUrCrArCrCrArGrCrCrArCr |
| | ArArCrGrArGrGrArUrUrArUrArCrCrArUrUrGrUrUrGrArArCrArGrUrArUrGrArArCrGrUrGrCr |
| | ArGrArArGrGrUrCrGrCrCrArUrArGrUrArCrCrGrGrUrGrGrUrArUrGrGrArUrGrArArCrUrGrU |
| | rArUrArArGrUrArArCrUrCrGrArGrArGrArUrCrCrGrGrCrUrGrCrUrArArCrArArArGrCrCrCrG |
| | rArArArGrGrArArGrCrUrGrArGrUrUrGrGrCrUrGrCrUrGrCrCrArCrCrGrCrUrGrArGrCrArArU |
| | rArArGrUrCrUrGrCrUrUrCrGrCrArCrCrArArArGrCrCmA*mU*mU*mA*mG*mC - |
| | `Product-892 mer 2’ |
| 104 | rA*rC*rU*rC*rC*rU*rC*rG*rG*rU*rG*rC*rC*rU*rC*rA - ‘5′primer N + 6_2′OH_PS’ |
| 105 | rA*rC*rU*rC*rC*rU*rC*rG*rG*rU*rG*rC*rC*rU*rC*rArArUrGrGrGrCrArGrCrArGrCrCr |
| | ArUrCrArUrCrArUrCrArUrCrArUrCrArCrArGrCrArGrCrGrGrCrCrUrGrGrUrGrCrCrGrCrGrCr |
| | GrGrCrArGrCrCrArUrArUrGrGrCrUrArGrCrArUrGrGrUrGrArGrCrArArArGrGrUrGrArArGrA |
| | rGrGrArUrArArUrArUrGrGrCrCrArUrCrArUrCrArArArGrArArUrUrCrArUrGrCrGrCrUrUrCrA |
| | rArArGrUrUrCrArUrArUrGrGrArArGrGrUrArGrCrGrUrUrArArUrGrGrCrCrArCrGrArArUrUr |
| | UrGrArArArUrUrGrArArGrGrUrGrArArGrGrCrGrArArGrGrUrCrGrUrCrCrGrUrArUrGrArArG |
| | rGrCrArCrCrCrArGrArCrCrGrCrArArArArCrUrGrArArArGrUrUrArCrCrArArArGrGrUrGrGrU |
| | rCrCrGrCrUrGrCrCrGrUrUrUrGrCrArUrGrGrGrArUrArUrUrCrUrGrArGrUrCrCrGrCrArGrUr |
| | UrUrArUrGrUrArUrGrGrUrArGrCrArArArGrCrCrUrArUrGrUrUrArArArCrArUrCrCrGrGrCrAr |
| | GrArUrArUrCrCrCrGrGrArUrUrArUrCrUrGrArArArCrUrGrArGrCrUrUrUrCrCrGrGrArArGrGr |
| | UrUrUrUrArArArUrGrGrGrArArCrGrUrGrUrGrArUrGrArArUrUrUrUrGrArArGrArUrGrGrUrG |
| | rGrUrGrUrUrGrUrUrArCrCrGrUrUrArCrArCrArGrGrArUrArGrCrArGrCrCrUrGrCrArGrGrAr |
| | UrGrGrUrGrArArUrUrUrArUrCrUrArUrArArArGrUrUrArArArCrUrGrCrGrUrGrGrCrArCrGrA |
| | rArUrUrUrUrCrCrGrArGrUrGrArUrGrGrUrCrCrGrGrUrUrArUrGrCrArGrArArArArArGrArCrC |
| | rArUrGrGrGrUrUrGrGrGrArArGrCrArArGrCrArGrCrGrArArCrGrUrArUrGrUrArUrCrCrGrGr |
| | ArArGrArUrGrGrCrGrCrArCrUrGrArArArGrGrUrGrArArArUrUrArArArCrArGrCrGrUrCrUrGr |
| | ArArGrCrUrGrArArArGrArUrGrGrCrGrGrUrCrArUrUrArUrGrArUrGrCrArGrArArGrUrUrArAr |
| | ArArCrCrArCrCrUrArCrArArArGrCrCrArArArArArGrCrCrGrGrUrUrCrArGrCrUrGrCrCrUrGr |
| | GrUrGrCrArUrArUrArArUrGrUrUrArArUrArUrCrArArArCrUrGrGrArUrArUrCrArCrCrArGrCr |
| | CrArCrArArCrGrArGrGrArUrUrArUrArCrCrArUrUrGrUrUrGrArArCrArGrUrArUrGrArArCrGr |
| | UrGrCrArGrArArGrGrUrCrGrCrCrArUrArGrUrArCrCrGrGrUrGrGrUrArUrGrGrArUrGrArArC |
| | rUrGrUrArUrArArGrUrArArCrUrCrGrArGrArGrArUrCrCrGrGrCrUrGrCrUrArArCrArArArGrC |
| | rCrCrGrArArArGrGrArArGrCrUrGrArGrUrUrGrGrCrUrGrCrUrGrCrCrArCrCrGrCrUrGrArGrC |
| | rArArUrArArGrUrCrUrGrCrUrUrCrGrCrArCrCrArArArGrCrCmA*mU*mU*mA*mG*mC - |
| | `Product-892 mer 3’ |
| 106 | rArCrUrCrCrUrCrGrGrUrGrCrCrUrCrArAΨrGrGrGrCrArGrCrArGrCrCrAΨrCrAΨrCrAΨrCrA |
| | ΨrCrAΨrCrArCrArGrCrArGrCrGrGrCrCΨrGrGΨrGrCrCrGrCrGrCrGrGrCrArGrCrCrAΨrAΨr |
| | GrGrCΨrArGrCrAΨrGrGΨrGrArGrCrArArArGrGΨrGrArArGrArGrGrAΨrArAΨrAΨrGrGrCr |
| | CrAΨrCrAΨrCrArArArGrArAΨΨrCrAΨrGrCrGrCΨΨrCrArArArGΨΨrCrAΨrAΨrGrGrArArG |
| | rGΨrArGrCrGΨΨrArAΨrGrGrCrCrArCrGrArAΨΨΨrGrArArAΨΨrGrArArGrGrGΨrArArGrG |
| | rCrGrArArGrGΨrCrGΨrCrCrGΨrAΨrGrArArGrGrCrArCrCrCrArGrArCrCrGrCrArArArArCΨr |
| | GrArArArGΨΨrArCrCrArArArGrGΨrGrGΨrCrCrGrCΨrGrCrCrGΨΨΨrGrCrAΨrGrGrGrAΨr |
| | AΨΨrCΨrGrArGΨrCrCrGrCrArGΨΨΨrAΨrGΨrAΨrGrGΨrArGrCrArArArGrCrCΨrAΨrGΨ |
| | ΨrArArArCrAΨrCrCrGrGrCrArGrAΨrAΨrCrCrCrGrGrAΨΨrAΨrCΨrGrArArArCΨrGrArGrC |
| | ΨΨΨrCrCrGrGrArArGrGΨΨΨΨrArArAΨrGrGrGrArArCrGΨrGΨrGrAΨrGrArAΨΨΨΨrGrA |
| | rArGrAΨrGrGΨrGrGΨrGΨΨrGΨΨrArCrCrGΨΨrArCrArCrArGrGrAΨArGrCrArGrCrCΨrG |
| | rCrArGrGrAΨrGrGΨrGrArAΨΨΨrAΨrCΨrAΨrArArArGΨΨrArArArCΨrGrCrGΨrGrGrCrAr |
| | CrGrArAΨΨΨΨrCrCrGrArGΨrGrAΨrGrGΨrCrCrGrGΨΨrAΨrGrCrArGrArArArArArGrArCr |
| | CrAΨrGrGrGΨΨrGrGrGrArArGrCrArArGrCrArGrCrGrArArCrGΨrAΨrGΨrAΨrCrCrGrGrAr |
| | ArGrAΨrGrGrCrGrCrArCΨrGrArArArGrGΨrGrArArAΨΨrArArArCrArGrCrGΨrCΨrGrArArG |
| | rCΨrGrArArArGrAΨrGrGrCrGrGΨrCrAΨΨrAΨrGrAΨrGrCrArGrArArGΨΨrArArArArCrCrA |
| | rCrCΨrArCrArArArGrCrCrArArArArArGrCrCrGrGΨΨrCrArGrCΨrGrCrCΨrGrGΨrGrCrAΨr |
| | AΨrArAΨrGYΨrArAΨrAΨrCrArArArCΨrGrGrAΨrAΨrCrArCrCrArGrCrCrArCrArArCrGrAr |
| | GrGrAΨΨrAΨrArCrCrAΨΨrGΨΨrGrArArCrArGΨrAΨrGrArArCrGΨrGrCrArGrArArGrGΨr |
| | CrGrCrCrAΨrArGΨrArCrCrGrGΨrGrGΨrAΨrGrGrAΨrGrArArCΨrGΨrAΨrArArGΨrArArC |
| | ΨrCrGrArGrArGrAΨrCrCrGrGrCΨrGrCΨrArArCrArArArGrCrCrCrGrArArArGrGrArArGrCΨr |
| | GrArGYΨrGrGrCΨrGrCΨrGrCrCrArCrCrGrCΨrGrArGrCrArAΨrArArGrUrCrUrGrCrUrUrCr |
| | GrCrArCrCrArArArGrCrCmA*mU*mU*mA*mG*mC - ‘Product-892 mer 4’ |
| 107 | rArCrUrCrCrUrCrGrGrUrGrCrCrUrCrArAm1ΨrGrGrGrCrArGrCrArGrCrCrAm1ΨrCrAm1Ψr |
| | CrAm1ΨrCrAm1ΨrCrAm1ΨrCrArCrArGrCrArGrCrGrGrCrCm1ΨrGrGm1ΨrGrCrCrGrCrGr |
| | CrGrGrCrArGrCrCrAm1ΨrAm1ΨrGrGrCm1ΨrArGrCrAm1ΨrGrGm1ΨrGrArGrCrArArArGr |
| | Gm1ΨrGrArArGrArGrGrAm1ΨrArAm1ΨrAm1ΨrGrGrCrCrAm1ΨrCrAm1ΨrCrArArArGrAr |
| | Am1Ψm1ΨrCrAm1ΨrGrCrGrCm1Ψm1ΨrCrArArArGm1Ψm1ΨrCrAm1ΨrAm1ΨrGrGrAr |
| | ArGrGm1ΨrArGrCrGm1Ψm1ΨrArAm1ΨrGrGrCrCrArCrGrArAm1Ψm1Ψm1ΨrGrArArAm |
| | 1Ψm1ΨrGrArArGrGm1ΨrGrArArGrGrCrGrArArGrGm1ΨrCrGm1ΨrCrCrGm1ΨrAm1ΨrGr |
| | ArArGrGrCrArCrCrCrArGrArCrCrGrCrArArArArCm1ΨrGrArArArGm1Ψm1ΨrArCrCrArArAr |
| | GrGm1ΨrGrGm1ΨrCrCrGrCm1ΨrGrCrCrGm1Ψm1Ψm1ΨrGrCrAm1ΨrGrGrGrAm1ΨrA |
| | m1Ψm1ΨrCm1ΨrGrArGm1ΨrCrCrGrCrArGm1Ψm1Ψm1ΨrAm1ΨrGm1ΨrAm1ΨrGrGm |
| | 1ΨrArGrCrArArArGrCrCm1ΨrAm1ΨrGm1Ψm1ΨrArArArCrAm1ΨrCrCrGrGrCrArGrAm1 |
| | ΨrAm1ΨrCrCrCrGrGrAm1Ψm1ΨrAm1ΨrCm1ΨrGrArArArCm1ΨrGrArGrCm1Ψm1Ψm1 |
| | ΨrCrCrGrGrArArGrGm1Ψm1Ψm1Ψm1ΨrArArAm1ΨrGrGrGrArArCrGm1ΨrGm1ΨrGrAm |
| | 1ΨrGrArAm1Ψm1Ψm1Ψm1ΨrGrArArGrAm1ΨrGrGm1YrGrGm1ΨrGm1Ψm1ΨrGm1Ψ |
| | m1ΨrArCrCrGm1Ψm1ΨrArCrArCrArGrGrAm1ΨrArGrCrArGrCrCm1ΨrGrCrArGrGrAm1Ψ |
| | rGrGm1ΨrGrArAm1Ψm1Ψm1ΨrAm1ΨrCm1ΨrAm1ΨrArArArGm1Ψm1ΨrArArArCm1Ψ |
| | rGrCrGm1ΨrGrGrCrArCrGrArAm1Ψm1Ψm1Ψm1ΨrCrCrGrArGm1ΨrGrAm1ΨrGrGm1Ψr |
| | CrCrGrGm1Ψm1ΨrAm1ΨrGrCrArGrArArArArArGrArCrCrAm1ΨrGrGrGm1Ψm1ΨrGrGrG |
| | rArArGrCrArArGrCrArGrCrGrArArCrGm1ΨrAm1ΨrGm1ΨrAm1ΨrCrCrGrGrArArGrAm1Ψ |
| | rGrGrCrGrCrArCm1ΨrGrArArArGrGm1ΨrGrArArAm1Ψm1ΨrArArArCrArGrCrGm1ΨrCm |
| | 1ΨrGrArArGrCm1ΨrGrArArArGrAm1ΨrGrGrCrGrGm1ΨrCrAm1Ψm1ΨrAm1ΨrGrAm1Ψr |
| | GrCrArGrArArGm1Ψm1ΨrArArArArCrCrArCrCm1ΨrArCrArArArGrCrCrArArArArArGrCrCr |
| | GrGm1Ψm1ΨrCrArGrCm1ΨrGrCrCm1ΨrGrGm1ΨrGrCrAm1YrAm1ΨrArAm1YrGm1Ψ |
| | m1ΨrArAm1ΨrAm1ΨrCrArArArCm1ΨrGrGrAm1ΨrAm1ΨrCrArCrCrArGrCrCrArCrArArCr |
| | GrArGrGrAm1Ψm1ΨrAm1ΨrArCrCrAm1Ψm1ΨrGm1Ψm1ΨrGrArArCrArGm1ΨrAm1Ψr |
| | GrArArCrGm1ΨrGrCrArGrArArGrGm1ΨrCrGrCrCrAm1ΨrArGm1ΨrArCrCrGrGm1ΨrGrG |
| | m1ΨrAm1ΨrGrGrAm1ΨrGrArArCm1ΨrGm1ΨrAm1ΨrArArGm1ΨrArArCm1ΨrCrGrArGr |
| | ArGrAm1ΨrCrCrGrGrCm1ΨrGrCm1ΨrArArCrArArArGrCrCrCrGrArArArGrGrArArGrCm1 |
| | ΨrGrArGm1Ψm1ΨrGrGrCm1ΨrGrCm1ΨrGrCrCrArCrCrGrCm1ΨrGrArGrCrArAm1ΨrArA |
| | rGrUrCrUrGrCrUrUrCrGrCrArCrCrArArArGrCrCmA*mU*mU*mA*mG*mC - ‘Product- |
| | 892 mer 5’ |
| 108 | rArCrUrCrCrUrCrGrGrUrGrCrCrUrCrArA*rU*rG*rG*rG*rC*rA*rG*rC*rA*rG*rC*rC*rA*r |
| | U*rC*rA*rU*rC*rA*rU*rC*rA*rU*rC*rA*rU*rC*rA*rC*rA*rG*rC*rA*rG*rC*rG*rG*rC |
| | *rC*rU*rG*rG*rU*rG*rC*rC*rG*rC*rG*rC*rG*rG*rC*rA*rG*rC*rC*rA*rU*rA*rU*rG* |
| | rG*rC*rU*rA*rG*rC*rA*rU*rG*rG*rU*rG*rA*rG*rC*rA*rA*rA*rG*rG*rU*rG*rA*rA*r |
| | G*rA*rG*rG*rA*rU*rA*rA*rU*rA*rU*rG*rG*rC*rC*rA*rU*rC*rA*rU*rC*rA*rA*rA*rG |
| | *rA*rA*rU*rU*rC*rA*rU*rG*rC*rG*rC*rU*rU*rC*rA*rA*rA*rG*rU*rU*rC*rA*rU*rA*r |
| | U*rG*rG*rA*rA*rG*rG*rU*rA*rG*rC*rG*rU*rU*rA*rA*rU*rG*rG*rC*rC*rA*rC*rG*rA |
| | *rA*rU*rU*rU*rG*rA*rA*rA*rU*rU*rG*rA*rA*rG*rG*rU*rG*rA*rA*rG*rG*rC*rG*rA* |
| | rA*rG*rG*rU*rC*rG*rU*rC*rC*rG*rU*rA*rU*rG*rA*rA*rG*rG*rC*rA*rC*rC*rC*rA*r |
| | G*rA*rC*rC*rG*rC*rA*rA*rA*rA*rC*rU*rG*rA*rA*rA*rG*rU*rU*rA*rC*rC*rA*rA*rA* |
| | rG*rG*rU*rG*rG*rU*rC*rC*rG*rC*rU*rG*rC*rC*rG*rU*rU*rU*rG*rC*rA*rU*rG*rG*r |
| | G*rA*rU*rA*rU*rU*rC*rU*rG*rA*rG*rU*rC*rC*rG*rC*rA*rG*rU*rU*rU*rA*rU*rG*rU |
| | *rA*rU*rG*rG*rU*rA*rG*rC*rA*rA*rA*rG*rC*rC*rU*rA*rU*rG*rU*rU*rA*rA*rA*rC*r |
| | A*rU*rC*rC*rG*rG*rC*rA*rG*rA*rU*rA*rU*rC*rC*rC*rG*rG*rA*rU*rU*rA*rU*rC*rU |
| | *rG*rA*rA*rA*rC*rU*rG*rA*rG*rC*rU*rU*rU*rC*rC*rG*rG*rA*rA*rG*rG*rU*rU*rU* |
| | rU*rA*rA*rA*rU*rG*rG*rG*rA*rA*rC*rG*rU*rG*rU*rG*rA*rU*rG*rA*rA*rU*rU*rU*r |
| | U*rG*rA*rA*rG*rA*rU*rG*rG*rU*rG*rG*rU*rG*rU*rU*rG*rU*rU*rA*rC*rC*rG*rU*r |
| | U*rA*rC*rA*rC*rA*rG*rG*rA*rU*rA*rG*rC*rA*rG*rC*rC*rU*rG*rC*rA*rG*rG*rA*rU |
| | *rG*rG*rU*rG*rA*rA*rU*rU*rU*rA*rU*rC*rU*rA*rU*rA*rA*rA*rG*rU*rU*rA*rA*rA* |
| | rC*rU*rG*rC*rG*rU*rG*rG*rC*rA*rC*rG*rA*rA*rU*rU*rU*rU*rC*rC*rG*rA*rG*rU*r |
| | G*rA*rU*rG*rG*rU*rC*rC*rG*rG*rU*rU*rA*rU*rG*rC*rA*rG*rA*rA*rA*rA*rA*rG*rA |
| | *rC*rC*rA*rU*rG*rG*rG*rU*rU*rG*rG*rG*rA*rA*rG*rC*rA*rA*rG*rC*rA*rG*rC*rG* |
| | rA*rA*rC*rG*rU*rA*rU*rG*rU*rA*rU****rC*rG*rG*rA*rA*rG*rA*rU*rG*rG*rC*rG*r |
| | C*rA*rC*rU*rG*rA*rA*rA*rG*rG*rU*rG*rA*rA*rA*rU*rU*rA*rA*rA*rC*rA*rG*rC*rG |
| | *rU*rC*rU*rG*rA*rA*rG*rC*rU*rG*rA*rA*rA*rG*rA*rU*rG*rG*rC*rG*rG*rU*rC*rA* |
| | rU*rU*rA*rU*rG*rA*rU*rG*rC*rA*rG*rA*rA*rG*rU*rU*rA*rA*rA*rA*rC*rC*rA*rC*rC |
| | *rU*rA*rC*rA*rA*rA*rG*rC*rC*rA*rA*rA*rA*rA*rG*rC*rC*rG*rG*rU*rU*rC*rA*rG*r |
| | C*rU*rG*rC*rC*rU*rG*rG*rU*rG*rC*rA*rU*rA*rU*rA*rA*rU*rG*rU*rU*rA*rA*rU*rA |
| | *rU*rC*rA*rA*rA*rC*rU*rG*rG*rA*rU*rA*rU*rC*rA*rC*rC*rA*rG*rC*rC*rA*rC*rA*r |
| | A*rC*rG*rA*rG*rG*rA*rU*rU*rA*rU*rA*rC*rC*rA*rU*rU*rG*rU*rU*rG*rA*rA*rC*rA |
| | *rG*rU*rA*rU*rG*rA*rA*rC*rG*rU*rG*rC*rA*rG*rA*rA*rG*rG*rU*rC*rG*rC*rC*rA* |
| | rU*rA*rG*rU*rA*rC*rC*rG*rG*rU*rG*rG*rU*rA*rU*rG*rG*rA*rU*rG*rA*rA*rC*rU*r |
| | G*rU*rA*rU*rA*rA*rG*rU*rA*rA*rC*rU*rC*rG*rA*rG*rA*rG*rA*rU*rC*rC*rG*rG*rC |
| | *rU*rG*rC*rU*rA*rA*rC*rA*rA*rA*rG*rC*rC*rC*rG*rA*rA*rA*rG*rG*rA*rA*rG*rC*r |
| | U*rG*rA*rG*rU*rU*rG*rG*rC*rU*rG*rC*rU*rG*rC*rC*rA*rC*rC*rG*rC*rU*rG*rA*rG |
| | *rC*rA*rA*rU*rA*rArGrUrCrUrGrCrUrUrCrGrCrArCrCrArArArGrCrCmA*mU*mU*mA*m |
| | G*mC - ‘Product-892 mer 6’ |

SEQ ID NO: 1
/5Biosg/GCTAATGGCTTTGGTGCGAAGCAGACTGAGGCACCGAGGAGT
SEQ ID NO: 2
ACTCCTCGGT
SEQ ID NO: 3
/5phos/AGTCTGCTTCGCACCAAAGCCATTAGC
SEQ ID NO: 4
/5phos/GTCTGCTTCGCACCAAAGCCATTAGC
SEQ ID NO: 5
/5Phos/GCACCAAAGC/Me-dC/ATTAGC
SEQ ID NO: 6
/5Phos/GC/Me-dC/ATTAGC
SEQ ID NO: 7
ACTCCTCGGTG
SEQ ID NO: 8
ACTCCTCGGTGC
SEQ ID NO: 9
ACTCCTCGGTGCC
SEQ ID NO: 10
ACTCCTCGGTGCCT
SEQ ID NO: 11
ACTCCTCGGTGCCTC
SEQ ID NO: 12
ACTCCTCGGTGCCTCA
SEQ ID NO: 13
ACTCCTCGGTGCCTCAG
SEQ ID NO: 14
ACTCCTCGGTGCCTCAGT
SEQ ID NO: 15
ACTCCTCGGTGCCTCAGTC
SEQ ID NO: 16
ACTCCTCGGTGCCTCAGTCT
SEQ ID NO: 17
ACTCCTCGGTGCCTCAGTCTG
SEQ ID NO: 18
ACTCCTCGGTGCCTCAGTCTGC
SEQ ID NO: 19
ACTCCTCGGTGCCTCAGTCTGCT
SEQ ID NO: 20
ACTCCTCGGTGCCTCAGTCTGCTT
SEQ ID NO: 21
ACTCCTCGGTGCCTCAGTCTGCTTC
SEQ ID NO: 22
ACTCCTCGGTGCCTCAGTCTGCTTCG
SEQ ID NO: 23
ACTCCTCGGTGCCTCAGTCTGCTTCGC
SEQ ID NO: 24
ACTCCTCGGTGCCTCAGTCTGCTTCGCA
SEQ ID NO: 25
ACTCCTCGGTGCCTCAGTCTGCTTCGCAC
SEQ ID NO: 26
ACTCCTCGGTGCCTCAGTCTGCTTCGCACC
SEQ ID NO: 27
ACTCCTCGGTGCCTCAGTCTGCTTCGCACCA
SEQ ID NO: 28
ACTCCTCGGTGCCTCAGTCTGCTTCGCACCAA
SEQ ID NO: 29
ACTCCTCGGTGCCTCAGTCTGCTTCGCACCAAA
SEQ ID NO: 30
ACTCCTCGGTGCCTCAGTCTGCTTCGCACCAAAG
SEQ ID NO: 31
ACTCCTCGGTGCCTCAGTCTGCTTCGCACCAAAGC
SEQ ID NO: 32
ACTCCTCGGTGCCTCAGTCTGCTTCGCACCAAAGCC
SEQ ID NO: 33
ACTCCTCGGTGCCTCAGTCTGCTTCGCACCAAAGCCA
SEQ ID NO: 34
ACTCCTCGGTGCCTCAGTCTGCTTCGCACCAAAGCCAT
SEQ ID NO: 35
ACTCCTCGGTGCCTCAGTCTGCTTCGCACCAAAGCCATT
SEQ ID NO: 36
ACTCCTCGGTGCCTCAGTCTGCTTCGCACCAAAGCCATTA
SEQ ID NO: 37
ACTCCTCGGTGCCTCAGTCTGCTTCGCACCAAAGCCATTAG
SEQ ID NO: 38
ACTCCTCGGTGCCTCAGTCTGCTTCGCACCAAAGCCATTAGC
SEQ ID NO: 39
MIVSDIEANALLESVTKFHCGVIYDYSTAEYVSYRPSDFGAYLDALEAEVARGGLIVFHNGHKYDVP
ALTKLAKLQLNREFHLPRENCIDTLVLSRLIHSNLKDTDMGLLRSGKLPGKRFGSHALEAWGYRLGEMKGEY
KDDFKRMLEEQGEEYVDGMEWWNFNEEMMDYNVQDVVVTKALLEKLLSDKHYFPPEIDFTDVGYTTFWS
ESLEAVDIEHRAAWLLAKQERNGFPFDTKAIEELYVELAARRSELLRKLTETFGSWYQPKGGTEMFCHPRTG
KPLPKYPRIKTPKVGGIFKKPKNKAQREGREPCELDTREYVAGAPYTPVEHVVFNPSSRDHIQKKLQEAGWV
PTKYTDKGAPVVDDEVLEGVRVDDPEKQAAIDLIKEYLMIQKRIGQSAEGDKAWLRYVAEDGKIHGSVNPN
GAVTGRATHAFPNLAQIPGVRSPYGEQCRAAFGAEHHLDGITGKPWVQAGIDASGLELRCLAHFMARFDNG
EYAHEILNGDIHTKNQIAAELPTRDNAKTFIYGFLYGAGDEKIGQIVGAGKERGKELKKKFLENTPAIAALRES
IQQTLVESSQWVAGEQQVKWKRRWIKGLDGRKVHVRSPHAALNTLLQSAGALICKLWIIKTEEMLVEKGLK
HGWDGDFAYMAWVHDEIQVGCRTEEIAQVVIETAQEAMRWVGDHWNFRCLLDTEGKMGPNWAICH
SEQ ID NO: 40
MIVLFVDFDYFYAQVEEVLNPSLKGKPVVVCVFSGRFEDSGAVATANYEARKFGVKAGIPIVEAKKIL
PNAVYLPMRKEVYQQVSSRIMNLLREYSEKIEIASIDEAYLDISDKVRDYREAYNLGLEIKNKILEKEKITVTVG
ISKNKVFAKIAADMAKPNGIKVIDDEEVKRLIRELDIADVPGIGNITAEKLKKLGINKLVDTLSIEFDKLKGMIG
EAKAKYLISLARDEYNEPIRTRVRKSIGRIVTMKRNSRNLEEIKPYLFRAIEESYYKLDKRIPKAIHVVAVTEDL
DIVSRGRTFPHGISKETAYSESVKLLQKILEEDERKIRRIGVRFSKFIEAIGLDKFFDT
SEQ ID NO: 41
MILDTDYITENGKPVIRVFKKENGEFKIEYDRTFEPYFYALLKDDSAIEDVKKVTAKRHGTVVKVKRA
EKVQKKFLGRPIEVWKLYFNHPQDVPAIRDRIRAHPAVVDIYEYDIPFAKRYLIDKGLIPMEGDEELTMLAFAI
ATLYHEGEEFGTGPILMISYADGSEARVITWKKIDLPYVDVVSTEKEMIKRFLRVVREKDPDVLITYNGDNFD
FAYLKKRCEELGIKFTLGRDGSEPKIQRMGDRFAVEVKGRIHFDLYPVIRRTINLPTYTLEAVYEAVFGKPKEK
VYAEEIAQAWESGEGLERVARYSMEDAKVTYELGREFFPMEAQLSRLIGQSLWDVSRSSTGNLVEWFLLRK
AYKRNELAPNKPDERELARRRGGYAGGYVKEPERGLWDNIVYLDFRSLYPSIIITHNVSPDTLNREGCKEYD
VAPEVGHKFCKDFPGFIPSLLGDLLEERQKIKRKMKATVDPLEKKLLDYRQRLIKILANSFYGYYGYAKARWY
CKECAESVTAWGREYIEMVIRELEEKFGFKVLYADTDGLHATIPGADAETVKKKAKEFLKYINPKLPGLLELEY
EGFYVRGFFVTKKKYAVIDEEGKITTRGLEIVRRDWSEIAKETQARVLEAILKHGDVEEAVRIVKEVTEKLSKY
EVPPEKLVIHEQITRDLRDYKATGPHVAVAKRLAARGVKIRPGTVISYIVLKGSGRIGDRAIPADEFDPTKHRY
DAEYYIENQVLPAVERILKAFGYRKEDLRYQKTKQVGLGAWLKVKGKK
SEQ ID NO: 42
MKEFYISIETVGNNIVERYIDENGKERTREVEYLPTMFRHCKEESKYKDIYGKNCAPQKFPSMKDAR
DWMKRMEDIGLEALGMNDFKLAYISDTYGSEIVYDRKFVRVANCDIEVTGDKFPDPMKAEYEIDAITHYDSI
DDRFYVFDLLNSMYGSVSKWDAKLAAKLDCEGGDEVPQEILDRVIYMPFDNERDMLMEYINLWEQKRPAIF
TGWNIEGFDVPYIMNRVKMILGERSMKRFSPIGRVKSKLIQNMYGSKEIYSIDGVSILDYLDLYKKFAFTNLP
SFSLESVAQHETKKGKLPYDGPINKLRETNHQRYISYNIIDVESVQAIDKIRGFIDLVLSMSYYAKMPFSGVMS
PIKTWDAIIFNSLKGEHKVIPQQGSHVKQSFPGAFVFEPKPIARRYIMSFDLTSLYPSIIRQVNISPETIRGQFK
VHPIHEYIAGTAPKPSDEYSCSPNGWMYDKHQEGIIPKEIAKVFFQRKDWKKKMFAEEMNAEAIKKIIMKGA
GSCSTKPEVERYVKFSDDFLNELSNYTESVLNSLIEECEKAATLANTNQLNRKILINSLYGALGNIHFRYYDLR
NATAITIFGQVGIQWIARKINEYLNKVCGTNDEDFIAAGDTDSVYVCVDKVIEKVGLDRFKEQNDLVEFMNQ
FGKKKMEPMIDVAYRELCDYMNNREHLMHMDREAISCPPLGSKGVGGFWKAKKRYALNVYDMEDKRFAEP
HLKIMGMETQQSSTPKAVQEALEESIRRILQEGEESVQEYYKNFEKEYRQLDYKVIAEVKTANDIAKYDDKG
WPGFKCPFHIRGVLTYRRAVSGLGVAPILDGNKVMVLPLREGNPFGDKCIAWPSGTELPKEIRSDVLSWIDH
STLFQKSFVKPLAGMCESAGMDYEEKASLDFLFG
SEQ ID NO: 43
MALEEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFANLWGRLEGEERLLWL
YREVERPLSAVLAHMEATGVRLDVAYLRALSLEVAEEIARLEAEVFRLAGHPFNLNSRDQLERVLFDELGLPAI
GKTEKTGKRSTSAAVLEALREAHPIVEKILQYRELTKLKSTYIDPLPDLIHPRTGRLHTRFNQTATATGRLSSS
DPNLQNIPVRTPLGQRIRRAFIAEEGWLLVALDYSQIELRVLAHLSGDENLIRVFQEGRDIHTETASWMFGV
PREAVDPLMRRAAKTINFGVLYGMSAHRLSQELAIPYEEAQAFIERYFQSFPKVRAWIEKTLEEGRRRGYVET
LFGRRRYVPDLEARVKSVREAAERMAFNMPVQGTAADLMKLAMVKLFPRLEEMGARMLLQVHDELVLEAPK
ERAEAVARLAKEVMEGVYPLAVPLEVEVGIGEDWLSAKE
SEQ ID NO: 44
MILDTDYITEDGKPVIRIFKKENGEFKIEYDRTFEPYFYALLKDDSAIEEVKKITAERHGTVVTVKRVE
KVQKKFLGRPVEVWKLYFTHPQDVPAIRDKIREHPAVIDIYEYDIPFAKRYLIDKGLVPMEGDEELKMLAFDIE
TLYHEGEEFAEGPILMISYADEEGARVITWKNVDLPYVDVVSTEREMIKRFLRVVKEKDPDVLITYNGDNFDF
AYLKKRCEKLGINFALGRDGSEPKIQRMGDRFAVEVKGRIHFDLYPVIRRTINLPTYTLEAVYEAVFGQPKEK
VYAEEITTAWETGENLERVARYSMEDAKVTYELGKEFLPMEAQLSRLIGQSLWDVSRSSTGNLVEWFLLRKA
YERNELAPNKPDEKELARRRQSYEGGYVKEPERGLWENIVYLDFRSLYPSIIITHNVSPDTLNREGCKEYDVA
PQVGHRFCKDFPGFIPSLLGDLLEERQKIKKKMKATIDPIERKLLDYRQRAIKILANSYYGYYGYARARWYCK
ECAESVTAWGREYITMTIKEIEEKYGFKVIYSDTDGFFATIPGADAETVKKKAMEFLKYINAKLPGALELEYEG
FYKRGFFVTKKKYAVIDEEGKITTRGLEIVRRDWSEIAKETQARVLEALLKDGDVEKAVRIVKEVTEKLSKYEV
PPEKLVIHEQITRDLKDYKATGPHVAVAKRLAARGVKIRPGTVISYIVLKGSGRIGDRAIPFDEFDPTKHKYDA
EYYIENQVLPAVERILRAFGYRKEDLRYQKTRQVGLSAWLKPKGT
SEQ ID NO: 45
MNIFNTNPFKAVSFVESAVKKALETSGYLIADCKYDGVRGNIVVDNVAEAAWLSRVSKFIPALEHLN
GFDKRWQQLLNDDRCIFPDGFMLDGELMVKGVDFNTGSGLLRTKWVKRDNMGFHLTNVPTKLTPKGREVI
DGKFEFHLDPKRLSVRLYAVMPIHIAESGEDYDVQNLLMPYHVEAMRSLLVEYFPEIEWLIAETYEVYDMDSL
TELYEEKRAEGHEGLIVKDPQGIYKRGKKSGWWKLKPECEADGIIQGVNWGTEGLANEGKVIGFSVLLETGR
LVDANNISRALMDEFTSNVKAHGEDFYNGWACQVNYMEATPDGSLRHPSFEKFRGTEDNPQEKM
SEQ ID NO: 46
/5Biosg/GCTAATGGCTTTGGTGCGAAGCAGACTGAGGCACCGAGGAGTTTT
SEQ ID NO: 47
CTAATGGCTTTGGTGCGAAGCAGACCTGATGACTGAGGCACCGAGGAGTTTTT
SEQ ID NO: 48
CTAATGGCTTTGGTGCGAAGCAGACCTCTTCACCTTTGCTCACCATTTGTAGTCCATCGGATATATCTCC
TTCGGATCCTGAGGCACCGAGGAGTTTTT
SEQ ID NO: 49
ACTCCfUfCfGfGfU
SEQ ID NO: 50
ACTCCTCGGTGCCTCA
SEQ ID NO: 51
rArCrUrCrCrUrCrGrGrUrGrCrCrUrCrA
SEQ ID NO: 52
mAmCmUmCmCmUmCmGmGmUmGmCmCmUmCmA
SEO ID NO: 53
mAmCmUmCmCmUmCmGmGmUmGmCmCrUrCrA
SEQ ID NO: 54
GTCTGCTTCGCACCAAAGCCATTAGC
SEQ ID NO: 55
ACTCCTCGGTGCCTCAGGATCCGAAGGAGATATATCCGATGGACTACAAATGGTGAGCAAAGGT
GAAGAG
SEQ ID NO: 56
MILDTDYITENGKPVIRVFKKENGEFKIEYDRTFEPYFYALLKDDSAIEDVKKVTAKRHGTVVKVKRA
EKVQKKFLGRPIEVWKLYFNHPQDVPAIRDRIRAHPAVVDIYEYDIPFAKRYLIDKGLIPMEGDEELTMLAFDI
ETLYHEGEEFGTGPILMISYADGSEARVITWKKIDLPYVDVVSTEKEMIKRFLRVVREKDPDVLITYNGDNFD
FAYLKKRCEELGIKFTLGRDGSEPKIQRMGDRFAVEVKGRIHFDLYPVIRRTINLPTYTLEAVYEAVFGKPKEK
VYAEEIAQAWESGEGLERVARYSMEDAKVTYELGREFFPMEAQLSRLIGQSLWDVSRSSTGNLVEWFLLRK
AYKRNELAPNKPDERELARRRGGYAGGYVKEPERGLWDNIVYLDFRSLYPSIIITHNVSPDTLNREGCKEYD
VAPEVGHKFCKDFPGFIPSLLGDLLEERQKIKRKMKATVDPLEKKLLDYRQRAIKILANSFYGYYGYAKARWY
CKECAESVTAWGREYIEMVIRELEEKFGFKVLYADTDGLHATIPGADAETVKKKAKEFLKYINPKLPGLLELEY
EGFYVRGFFVTKKKYAVIDEEGKITTRGLEIVRRDWSEIAKETQARVLEAILKHGDVEEAVRIVKEVTEKLSKY
EVPPEKLVIHEQITRDLRDYKATGPHVAVAKRLAARGVKIRPGTVISYIVLKGSGRIGDRAIPADEFDPTKHRY
DAEYYIENQVLPAVERILKAFGYRKEDLRYQKTKQVGLGAWLKVKGKK
SEQ ID NO: 57
MILDTDYITEDGKPVIRIFKKENGEFKIDYDRNFEPYIYALLKDDSAIEDVKKITAERHGTTVRVVRA
EKVKKKFLGRPIEVWKLYFTHPQDQPAIRDKIKEHPAVVDIYEYDIPFAKRYLIDKGLIPMEGDEELKMLAFAI
ATLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCGEYDV
APQVGHKFCKDFPGFIPSLLGDLLEERQKVKKKMKATIDPIEKKLLDYRQRLIGILANSFYGYYGYAKARWYC
KECAESVTAWGRQYLETTIREIEEKFGFKVLYADTDGFFATIPGADAETVKKKAKEFLDYINAKLPGLLELEYE
GFYKRGFFVTKKKYAVIDEEDKITTVGLEIVRVDWSEIAKETQARVLEAILKHGDVEEAVRIVKEVTEKLSKYE
VPPEKLVIYEQITRDLKDYKATGPHVAVAKRLAARGIKIRPGTVISYIVLKGSGRIGDRAIPFDEFDPAKHRYD
AEYYIENQVLPAVERILRAFGYRKEDLRYQKTRQVGLGAWLKPKT
SEQ ID NO: 58
MILDTDYITEDGKPVIRIFKKENGEFKIDYDRNFEPYIYALLKDDSAIEDVKKITAERHGTTVRVVRA
EKVKKKFLGRPIEVWKLYFTHPQDVPAIRDKIKEHPAVVDIYEYDIPFAKRYLIDKGLIPMEGDEELKMLAFDI
ETLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCEEYDV
APQVGHKFCKDFPGFIPSLLGDLLEERQKVKKKMKATIDPIEKKLLDYRQRLIKILANSFYGYYGYAKARWYC
KECAESVTAWGRQYIETTIREIEEKFGFKVLYADTDGFFATIPGADAETVKKKAKEFLDYINAKLPGLLELEYE
GFYKRGFFATKKKYAVIDEEDKITTRGLKMVRRDWSEIAKETQARVLEAILKHGDVEEAVRIVKEVTEKLSKY
EVPPEQLVIYQPITKQLHDYRARGPHVSVAKRLAARGIKIRPGTVISYIVPKGSGRIGDRAIPFDEFDPAKHKY
DAGYYIENQVLPAVERILRAFGYRKEDLRYQKTRQVGLGAWLKPKT
SEQ ID NO: 59
MALEEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFAYLWGRLEGEERLLWL
YREVERPLSAVLAHMEATGVRLDVAYLRALSLEVAEEIARLEAEVFRLAGHPFNLNSRDQLERVLFDELGLPAI
GKTEKTGKRSTSAAALEALREAHPIVEKILQYRELTKLKSTYIDPLPDLIHPRTGRLHTRFNQTATATGRLSSS
DPNLQNIPVRTPLGQRIRRAFIAEEGWLLVALDYSQEGLRVLAHLSGDENLIRVFQEGRDIHTETASWMFGV
PREAVNPMMRRAAKTINFGVLYGMSAHRLSQVLAIPYEEAQAFIERYFQSFPKVRAWIEKTLEEGRRRGYVE
TLFGRRRYVPDLEARVKSVRNAAERRAFNMPVQGTAADLMKLAMVKLFPRLEEMGARMLLQVHDELVLEAP
KERAEAVARLAKEVMEGVYPLAVPLEVEVGIGEDWLSAKE
SEQ ID NO: 60
MILDTDYITENGKPVIRVFKKENGEFKIEYDRTFEPYFYALLKDDSAIEDVKKVTAKRHGTVVKVKRA
EKVQKKFLGRPIEVWKLYFNHPQDQPAIRDRIRAHPAVVDIYEYDIPFAKRYLIDKGLIPMEGDEELTMLAFAI
ATLYHEGEEFGTGPILMISYADGSEARVITWKKIDLPYVDVVSTEKEMIKRFLRVVREKDPDVLITYNGDNFD
FAYLKKRCEELGIKFTLGRDGSEPKIQRMGDRFAVEVKGRIHFDLYPVIRRTINLPTYTLEAVYEAVFGKPKEK
VYAEEIAQAWESGEGLERVARYSMEDAKVTYELGREFFPMEAQLSRLIGQSLWDVSRSSTGNLVEWFLLRK
AYKRNELAPNKPDERELARRRGGYAGGYVKEPERGLWDNIVYLDFRSLGPSIIITHNVSPDTLNREGCKEYD
VAPEVGHKFCKDFPGFIPSLLGDLLEERQKIKRKMKATVDPLEKKLLDYRQRLIKILANSFYGYYGYAKARWY
CKECAESVTAWGREYIEMVIRELEEKFGFKVLYADTDGLHATIPGADAETVKKKAKEFLKYINPKLPGLLELEY
EGFYVRGFFVTKKKYAVIDEEGKITTRGLEIVRRDWSEIAKETQARVLEAILKHGDVEEAVRIVKEVTEKLSKY
EVPPEKLVIHKQITRDLRDYKATGPHVAVAKRLAARGVKIRPGTVISYIVLKGSGRIGDRAIPADEFDPTKHRY
DAEYYIENQVLPAVERILKAFGYRKEDLRYQKTKQVGLGAWLKVKGKK
SEQ ID NO: 61
MILDTDYITENGKPVIRVFKKENGEFKIEYDRTFEPYFYALLKDDSAIEDVKKVTAKRHGTVVKVKRA
EKVQKKFLGRPIEVWKLYFNHPQDVPAIRDRIRAHPAVVDIYEYDIPFAKRYLIDKGLIPMEGDEELTMLAFAI
ATLYHEGEEFGTGPILMISYADGSEARVITWKKIDLPYVDVVSTEKEMIKRFLRVVREKDPDVLITYNGDNFD
FAYLKKRCEELGIKFTLGRDGSEPKIQRMGDRFAVEVKGRIHFDLYPVIRRTINLPTYTLEAVYEAVFGKPKEK
VYAEEIAQAWESGEGLERVARYSMEDAKVTYELGREFFPMEAQLSRLIGQSLWDVSRSSTGNLVEWFLLRK
AYKRNELAPNKPDERELARRRGGYAGGYVKEPERGLWDNIVYLDFRSLYPSIIITHNVSPDTLNREGCKEYD
VAPEVGHKFCKDFPGFIPSLLGDLLEERQKIKRKMKATVDPLEKKLLDYRQRLIKILANSFYGYYGYAKARWY
CKECAESVTAWGREYIEMVIRELEEKFGFKVLYADTDGLRATIPGADAETVKKKAKEFLKYINPKLPGLLELEY
EGFYVRGFFVTKKKYAVIDEEGKITTRGLEIVRRDWSEIAKETQARVLEAILKHGDVEEAVRIVKEVTEKLSKY
EVPPEKLVIHEQITRDLRDYKATGPHVAVAKRLAARGVKIRPGTVISYIVLKGSGRIGDRAIPADEFDPTKHRY
DAEYYIENQVLPAVERILKAFGYRKEDLRYQKTKQVGLGAWLKVKGKK
SEQ ID NO: 62
MILDTDYITEDGKPVIRIFKKENGEFKIEYDRTFEPYFYALLKDDSAIEEVKKITAERHGTVVTVKRVE
KVQKKFLGRPVEVWKLYFTHPQDVPAIRDKIREHPAVIDIYEYDIPFAKRYLIDKGLVPMEGDEELKMLAFDIE
TLYHEGEEFAEGPILMISYADEEGARVITWKNVDLPYVDVVSTEREMIKRFLRVVKEKDPDVLITYDGDNFDF
AYLKKRCEKLGINFALGRDGSEPKIQRMGDRFAVEVKGRIHFDLYPVIRRTINLPTYTLEAVYEAVFGQPKEK
VYAEEITTAWETGENLERVARYSMEDAKVTYELGKEFLPMEAQLSRLIGQSLWDVSRSSTGNLVEWFLLRKA
YERNELAPNKPDEKELARRRQSYEGGYVKEPERGLWENIVYLDFRSLGPSIIITHNVSPDTLNREGCKEYDVA
PQVGHRFCKDFPGFIPSLLGDLLEERQKIKKKMKATIDPIERKLLDYRQRLIKILANSYYGYYGYARARWYCK
ECAESVTAWGREYITMTIKEIEEKYGFKVIYSDTDGFFATIPGADAETVKKKAMEFLKYINAKLPGALELEYEG
FYKRGFFVTKKKYAVIDEEGKITTRGLEIVRRNWSEIAKETQARVLEALLKDGDVEKAVRIVKEVTEKLSKYEV
PPEKLVIHKQITRDLKDYKATGPHVAVAKRLAARGVKIRPGTVISYIVLKGSGRIGDRAIPFDEFDPTKHKYDA
EYYIENQVLPAVERILRAFGYRKEDLRYQKTRQVGLSAWLKPKGT
SEQ ID NO: 63
MILDTDYITEDGKPVIRIFKKENGEFKIDYDRNFEPYIYALLKDDSAIEDVKKITAERHGTTVRVVRA
EKVKKKFLGRPIEVWKLYFTHPQDQPAIRDKIKEHPAVVDIYEYDIPFAKRYLIDKGLIPMEGDEELKMLAFAI
ATLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCGEYDV
APQVGHKFCKDFPGFIPSLLGPLLEERQKVKKKMKATIDPIEKKLLDYRQRLIGILANSFYGYYGYAKARWYC
KECAESVTAWGRQYLETTIREIEEKFGFKVLYADTDGFFATIPGADAETVKKKAKEFLDYINAKLPGLLELEYE
GFYKRGFFVTKKKYAVIDEEDKITTVGLEIVRVDWSEIAKETQARVLEAILKHGDVEEAVRIVKEVTEKLSKYE
VPPEKLVIYEQITRDLKDYKATGPHVAVAKRLAARGIKIRPGTVISYIVLKGSGRIGDRAIPFDEFDPAKHRYD
AEYYIENQVLPAVERILRAFGYRKEDLRYQKTRQVGLGAWLKPKT
SEQ ID NO: 64
MILDTDYITKDGKPIIRIFKKENGEFKIELDPHFQPYIYALLKDDSAIEEIKAIKGERHGKTVRVLDAV
KVRKKFLGREVEVWKLIFEHPQDVPAMRGKIREHPAVVDIYEYDIPFAKRYLIDKGLIPMEGDEELKLLAFDIE
TFYHEGDEFGKGEIIMISYADEEEARVITWKNIDLPYVDVVSNEREMIKRFVQVVKEKDPDVIITYNGDNFDL
PYLIKRAEKLGVRLVLGRDKEHPEPKIQRMGDSFAVEIKGRIHFDLFPVVRRTINLPTYTLEAVYEAVLGKTKS
KLGAEEIAAIWETEESMKKLAQYSMEDARATYELGKEFFPMEAELAKLIGQSVWDVSRSSTGNLVEWYLLRV
AYARNELAPNKPDEEEYKRRLRTTYLGGYVKEPEKGLWENIIYLDFRSLYPSIIVTHNVSPDTLEKEGCKNYD
VAPIVGYRFCKDFPGFIPSILGDLIAMRQDIKKKMKSTIDPIEKKMLDYRQRAIKLLANSYYGYMGYPKARWY
SKECAESVTAWGRHYIEMTIREIEEKFGFKVLYADTDGFYATIPGEKPELIKKKAKEFLNYINSKLPGLLELEYE
GFYLRGFFVTKKRYAVIDEEGRITTRGLEVVRRDWSEIAKETQAKVLEAILKEGSVEKAVEVVRDVVEKIAKY
RVPLEKLVIHEQITRDLKDYKAIGPHVAIAKRLAARGIKVKPGTIISYIVLKGSGKISDRVILLTEYDPRKHKYD
PDYYIENQVLPAVLRILEAFGYRKEDLRYQSSKQTGLDAWLKR
SEQ ID NO: 65
MALEEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFANLWGRLEGEERLLWL
YREVERPLSAVLAHMEATGVRLDVAYLRALSLEVAEEIARLEAEVFRLAGHPFNLNSRDQLERVLFDELGLPAI
GKTEKTGKRSTSAAVLEALREAHPIVEKILQYRELTKLKSTYIDPLPDLIHPRTGRLHTRFNQTATATGRLSSS
DPNLQNIPVRTPLGQRIRRAFIAEEGWLLVALDYSQEGLRVLAHLSGDENLIRVFQEGRDIHTETASWMFGV
PREAVDPLMRRAAKTINFGVLYGMSAHRLSQELAIPYEEAQAFIERYFQSFPKVRAWIEKTLEEGRRRGYVET
LFGRRRYVPDLEARVKSVRKRAERMAFNMPVQGTAADLMKLAMVKLFPRLEEMGARMLLQVHDELVLEAPK
ERAEAVARLAKEVMEGVYPLAVPLEVEVGIGEDWLSAKE
SEQ ID NO: 66
MILDTDYITENGKPVIRVFKKENGEFKIEYDRTFEPYFYALLKDDSAIEDVKKVTAKRHGTVVKVKRA
EKVQKKFLGRPIEVWKLYFNHPQDVPAIRDRIRAHPAVVDIYEYDIPFAKRYLIDKGLIPMEGDEELTMLAFAI
ATLYHEGEEFGTGPILMISYADGSEARVITWKKIDLPYVDVVSTEKEMIKRFLRVVREKDPDVLITYNGDNFD
FAYLKKRCEELGIKFTLGRDGSEPKIQRMGDRFAVEVKGRIHFDLYPVIRRTINLPTYTLEAVYEAVFGKPKEK
VYAEEIAQAWESGEGLERVARYSMEDAKVTYELGREFFPMEAQLSRLIGQSLWDVSRSSTGNLVEWFLLRK
AYKRNELAPNKPDERELARRRGGYAGGYVKEPERGLWDNIVYLDFRSLYPSIIITHNVSPDTLNREGCKEYD
VAPEVGHKFCKDFPGFIPSLLGDLLEERQKIKRKMKATVDPLEKKLLDYRQRLIKILANSFYGYYGYAKARWY
CKECAESVTAWGREYIEMVIRELEEKFGFKVLYADTDGLHATIPGADAETVKKKAKEFLKYINPKLPGLLELEY
EGFYVRGFFVTKKKYAVIDEEGKITTRGLEIVRRDWSEIAKETQARVLEAILKHGDVEEAVRIVKEVTEKLSKY
EVPPEKLVIHEQITRDLRDYKATGPHVAVAKRLAARGVKIRPGTVISYIVLKGSGRIGDRAIPADEFDPTKHRY
DAEYYIENQVLPAVERILKAFGYRKEDLRYQKTKQVGLGAWLKVKGKK
SEQ ID NO: 67
MILDTDYITEDGKPVIRIFKKENGEFKIEYDRTFEPYFYALLKDDSAIEEVKKITAERHGTVVTVKRVE
KVQKKFLGRPVEVWKLYFTHPQDVPAIRDKIREHPAVIDIYEYDIPFAKRYLIDKGLVPMEGDEELKMLAFAIA
TLYHEGEEFAEGPILMISYADEEGARVITWKNVDLPYVDVVSTEREMIKRFLRVVKEKDPDVLITYNGDNFDF
AYLKKRCEKLGINFALGRDGSEPKIQRMGDRFAVEVKGRIHFDLYPVIRRTINLPTYTLEAVYEAVFGQPKEK
VYAEEITTAWETGENLERVARYSMEDAKVTYELGKEFLPMEAQLSRLIGQSLWDVSRSSTGNLVEWFLLRKA
YERNELAPNKPDEKELARRRQSYEGGYVKEPERGLWENIVYLDFRSLYPSIIITHNVSPDTLNREGCKEYDVA
PQVGHRFCKDFPGFIPSLLGDLLEERQKIKKKMKATIDPIERKLLDYRQRRIKILANSYYGYYGYARARWYCK
ECAESVTAWGREYITMTIKEIEEKYGFKVIYSDTDGFFATIPGADAETVKKKAMEFLKYINAKLPGALELEYEG
FYKRGFFVTKKKYAVIDEEGKITTRGLEIVRRDWSEIAKETQARVLEALLKDGDVEKAVRIVKEVTEKLSKYEV
PPEKLVIHIQITRDLKDYKATGPHVAVAKRLAARGVKIRPGTVISYIVLKGSGRIGDRAIPFDEFDPTKHKYDA
EYYIENQVLPAVERILRAFGYRKEDLRYQKTRQVGLSAWLKPKGT
SEQ ID NO: 68
MILDTDYITEDGKPVIRIFKKENGEFKIDYDRNFEPYIYALLKDDSAIEDVKKITAERHGTTVRVVRA
EKVKKKFLGRPIEVWKLYFTHPQDQPAIRDKIKEHPAVVDIYEYDIPFAKRYLIDKGLIPMEGDEELKMLAFAI
ATLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCEEYDV
APQVGHKFCKDFPGFIPSLLGDLLEERQKVKKKMKATIDPIEKKLLDYRQRLIKILANSFYGYYGYAKARWYC
KECAESVTAWGRQYIETTIREIEEKFGFKVLYADTDGFFATIPGADAETVKKKAKEFLDYINAKLPGLLELEYE
GFYKRGFFVTKKKYAVIDEEDKITTRGLEIVRRDWSEIAKETQARVLEAILKHGDVEEAVRIVKEVTEKLSKYE
VPPEKLVIYEQITRDLKDYKATGPHVAVAKRLAARGIKIRPGTVISYIVLKGSGRIGDRAIPFDEFDPAKHKYD
AEYYIENQVLPAVERILRAFGYRKEDLRYQKTRQVGLGAWLKPKT
SEQ ID NO: 69
MILDTDYITKDGKPIIRIFKKENGEFKIELDPHFQPYIYALLKDDSAIEEIKAIKGERHGKTVRVLDAV
KVRKKFLGREVEVWKLIFEHPQDVPAMRGKIREHPAVVDIYEYDIPFAKRYLIDKGLIPMEGDEELKLLAFAIA
TFYHEGDEFGKGEIIMISYADEEEARVITWKNIDLPYVDVVSNEREMIKRFVQVVKEKDPDVIITYNGDNFDL
PYLIKRAEKLGVRLVLGRDKEHPEPKIQRMGDSFAVEIKGRIHFDLFPVVRRTINLPTYTLEAVYEAVLGKTKS
KLGAEEIAAIWETEESMKKLAQYSMEDARATYELGKEFFPMEAELAKLIGQSVWDVSRSSTGNLVEWYLLRV
AYARNELAPNKPDEEEYKRRLRTTYLGGYVKEPEKGLWENIIYLDFRSLVPSIIVTHNVSPDTLEKEGCKNYD
VAPIVGYRFCKDFPGFIPSILGDLIAMRQDIKKKMKSTIDPIEKKMLDYRQRLIKLLANSYYGYMGYPKARWY
SKECAESVTAWGRHYIEMTIREIEEKFGFKVLYADTDGFYATIPGEKPELIKKKAKEFLNYINSKLPGLLELEYE
GFYLRGFFVTKKRYAVIDEEGRITTRGLEVVRRDWSEIAKETQAKVLEAILKEGSVEKAVEVVRDVVEKIAKY
RVPLEKLVIHEQITRDLKDYKAIGPHVAIAKRLAARGIKVKPGTIISYIVLKGSGKISDRVILLTEYDPRKHKYD
PDYYIENQVLPAVLRILEAFGYRKEDLRYQSSKQTGLDAWLKR
SEQ ID NO: 70
MALEEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFANLWGRLEGEERLLWL
YREVERPLSAVLAHVEATGVRLDVAYLRALSLEVAEEIARLEAEVFRLAGHPFNLNSRDQLERVLFDELGLPAI
GKTEKTGKRSTSAAVLEALREAHAIVEKILQYRELTKLKSTYIDPLPELIHPRTGRLHTRFNQTATATGRLSSS
DPNLQNIPVRTPLGQRIRRAFIAEEGWLLVALDYSQIELRVLAHLSGDENLIRVFQEGRDIHTETASWMFGV
PREAVDPLMRRAAKTINFGVLYGMSAHRLSQELAIPYEEAQAFIERYFQSFPKVRAWIEKTLEEGRRRGYVET
LFGRRRYVPDLEARVKSVREAAERMAFNMPVQGTAADLMKLAMVKLFPRLEEMGARMLLQVHDELVLEAPK
ERAEAVARLAKEVMEGVYPLAVPLEVEVGIGEDWLSAKV
SEQ ID NO: 71
MILDTDYITENGKPVIRVFKKENGEFKIEYDRTFEPYFYALLKDDSAIEDVKKVTAKRHGTVVKVKRA
EKVQKKFLGRPIEVWKLYFNHPQDVPAIRDRIRAHPAVVDIYEYDIPFAKRYLIDKGLIPMEGDEELTMLAFAI
ATLYHEGEEFGTGPILMISYADGSEARVITWKKIDLPYVDVVSTEKEMIKRFLRWVREKDPDVLITYNGDNFD
FAYLKKRCEELGIKFTLGRDGSEPKIQRMGDRFAVEVKGRIHFDLYPVIRRTINLPTYTLEAVYEAVFGKPKEK
VYAEEIAQAWESGEGLERVARYSMEDAKVTYELGREFFPMEAQLSRLIGQSLWDVSRSSTGNLVEWFLLRK
AYKRNELAPNKPDERELARRRGGYAGGYVKEPERGLWDNIVYLDFRSLYPSIIITHNVSPDTLNREGCKEYD
VAPEVGHKFCKDFPGFIPSLLGDLLEERQKIKRKMKATVDPLEKKLLDYRQRRIKILANSFYGYYGYAKARWY
CKECAESVTAWGREYIEMVIRELEEKFGFKVLYADTDGLHATIPGADAETVKKKAKEFLKYINPKLPGLLELEY
EGFYVRGFFVTKKKYAVIDEEGKITTRGLEIVRRDWSEIAKETQARVLEAILKHGDVEEAVRIVKEVTEKLSKY
EVPPEKLVIHEQITRDLRDYKATGPHVAVAKRLAARGVKIRPGTVISYIVLKGSGRIGDRAIPADEFDPTKHRY
DAEYYIENQVLPAVERILKAFGYRKEDLRYQKTKQVGLGAWLKVKGKK
SEQ ID NO: 72
MILDTDYITEDGKPVIRIFKKENGEFKIEYDRTFEPYFYALLKDDSAIEEVKKITAERHGTVVTVKRVE
KVQKKFLGRPVEVWKLYFTHPQDQPAIRDKIREHPAVIDIYEYDIPFAKRYLIDKGLVPMEGDEELKMLAFAI
ATLYHEGEEFAEGPILMISYADEEGARVITWKNVDLPYVDVVSTEREMIKRFLRVVKEKDPDVLITYNGDNFD
FAYLKKRCEKLGINFALGRDGSEPKIQRMGDRFAVEVKGRIHFDLYPVIRRTINLPTYTLEAVYEAVFGQPKEK
VYAEEITTAWETGENLERVARYSMEDAKVTYELGKEFLPMEAQLSRLIGQSLWDVSRSSTGNLVEWFLLRKA
YERNELAPNKPDEKELARRRQSYEGGYVKEPERGLWENIVYLDFRSLGPSIIITHNVSPDTLNREGCKEYDVA
PQVGHRFCKDFPGFIPSLLGDLLEERQKIKKKMKATIDPIERKLLDYRQRLIKILANSYYGYYGYARARWYCK
ECAESVTAWGREYITMTIKEIEEKYGFKVIYSDTDGFFATIPGADAETVKKKAMEFLKYINAKLPGALELEYEG
FYKRGFFVTKKKYAVIDEEGKITTRGLEIVRRDWSEIAKETQARVLEALLKDGDVEKAVRIVKEVTEKLSKYEV
PPEKLVIHKQITRDLKDYKATGPHVAVAKRLAARGVKIRPGTVISYIVLKGSGRIGDRAIPFDEFDPTKHKYDA
EYYIENQVLPAVERILRAFGYRKEDLRYQKTRQVGLSAWLKPKGT
SEQ ID NO: 73
MILDTDYITEDGKPVIRIFKKENGEFKIDYDRNFEPYIYALLKDDSAIEDVKKITAERHGTTVRVVRA
EKVKKKFLGRPIEVWKLYFTHPQDQPAIRDKIKEHPAVVDIYEYDIPFAKRYLIDKGLIPMEGDEELKMLAFAI
ATLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCEEYDV
APQVGHKFCKDFPGFIPSLLGDLLEERQKVKKKMKATIDPIEKKLLDYRQRLIKILANSFYGYYGYAKARWYC
KECAESVTAWGRQYIETTIREIEEKFGFKVLYADTDGFFATIPGADAETVKKKAKEFLDYINAKLPGLLELEYE
GFYKRGFFATKKKYAVIDEEDKITTRGLKMVRRDWSEIAKETQARVLEAILKHGDVEEAVRIVKEVTEKLSKY
EVPPEQLVIYQPITKQLHDYRARGPHVSVAKRLAARGIKIRPGTVISYIVPKGSGRIGDRAIPFDEFDPAKHKY
DAGYYIENQVLPAVERILRAFGYRKEDLRYQKTRQVGLGAWLKPKT
SEQ ID NO: 74
MILDTDYITKDGKPIIRIFKKENGEFKIELDPHFQPYIYALLKDDSAIEEIKAIKGERHGKTVRVLDAV
KVRKKFLGREVEVWKLIFEHPQDVPAMRGKIREHPAVVDIYEYDIPFAKRYLIDKGLIPMEGDEELKLLAFAIA
TFYHEGDEFGKGEIIMISYADEEEARVITWKNIDLPYVDVVSNEREMIKRFVQVVKEKDPDVIITYNGDNFDL
PYLIKRAEKLGVRLVLGRDKEHPEPKIQRMGDSFAVEIKGRIHFDLFPVVRRTINLPTYTLEAVYEAVLGKTKS
KLGAEEIAAIWETEESMKKLAQYSMEDARATYELGKEFFPMEAELAKLIGQSVWDVSRSSTGNLVEWYLLRV
AYARNELAPNKPDEEEYKRRLRTTYLGGYVKEPEKGLWENIIYLDFRSLVPSIIVTHNVSPDTLEKEGCKNYD
VAPIVGYRFCKDFPGFIPSILGDLIAMRQDIKKKMKSTIDPIEKKMLDYRQRAIKLLANSYYGYMGYPKARWY
SKECAESVTAWGRHYIEMTIREIEEKFGFKVLYADTDGFYATIPGEKPELIKKKAKEFLNYINSKLPGLLELEYE
GFYLRGFFVTKKRYAVIDEEGRITTRGLEVVRRDWSEIAKETQAKVLEAILKEGSVEKAVEVVRDVVEKIAKY
RVPLEKLVIHEQITRDLKDYKAIGPHVAIAKRLAARGIKVKPGTIISYIVLKGSGKISDRVILLTEYDPRKHKYD
PDYYIENQVLPAVLRILEAFGYRKEDLRYQSSKQTGLDAWLKR
SEQ ID NO: 75
MALEEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFANLWGRLEGEERLLWL
YREVERPLSAVLAHMEATGVRLDVAYLRALSLEVAEEIARLEAEVFRLAGHPFNLNSRDQLERVLFDELGLPAI
GKTEKTGKRSTSAAVLEALREAHPIVEKILQYRELTKLKSTYIDPLPDLIHPRTGRLHTRFNQTATATGRLSSS
DPNLQNIPVRTPLGQRIRRAFIAEVGWLLVVLDYSQMGLRVLAHLSGDENLIRVFQEGRDIHTETASWMFGV
PREAVDPLMRRAAKTINFGVLYGMSAHRLSQELAIPYEEAQAFIERYFQSFPKVRAWIEKTLEEGRRRGYVET
LFGRRRYVPDLEARVKSVREAAERMAFNMPVQGTAADLMKLAMVKLFPRLEEMGARMLLQVHDELVLEAPK
ERAEAVARLAKEVMEGVYPLAVPLEVEVGIGEDWLSAKE
SEQ ID NO: 76
CTAATGGCTTTGGTGCGAAGCAGACTTATTGCTCAGCGGTGGCAGCAGCCAACTCAGCTTCCTT
TCGGGCTTTGTTAGCAGCCGGATCTCTCGAGTTACTTATACAGTTCATCCATACCACCGGTACTATGGCG
ACCTTCTGCACGTTCATACTGTTCAACAATGGTATAATCCTCGTTGTGGCTGGTGATATCCAGTTTGATA
TTAACATTATATGCACCAGGCAGCTGAACCGGCTTTTTGGCTTTGTAGGTGGTTTTAACTTCTGCATCAT
AATGACCGCCATCTTTCAGCTTCAGACGCTGTTTAATTTCACCTTTCAGTGCGCCATCTTCCGGATACATA
CGTTCGCTGCTTGCTTCCCAACCCATGGTCTTTTTCTGCATAACCGGACCATCACTCGGAAAATTCGTGC
CACGCAGTTTAACTTTATAGATAAATTCACCATCCTGCAGGCTGCTATCCTGTGTAACGGTAACAACACC
ACCATCTTCAAAATTCATCACACGTTCCCATTTAAAACCTTCCGGAAAGCTCAGTTTCAGATAATCCGGGA
TATCTGCCGGATGTTTAACATAGGCTTTGCTACCATACATAAACTGCGGACTCAGAATATCCCATGCAAA
CGGCAGCGGACCACCTTTGGTAACTTTCAGTTTTGCGGTCTGGGTGCCTTCATACGGACGACCTTCGCCT
TCACCTTCAATTTCAAATTCGTGGCCATTAACGCTACCTTCCATATGAACTTTGAAGCGCATGAATTCTTT
GATGATGGCCATATTATCCTCTTCACCTTTGCTCACCATGCTAGCCATATGGCTGCCGCGCGGCACCAGG
CCGCTGCTGTGATGATGATGATGATGGCTGCTGCCCATTGAGGCACCGAGGAGTTTTT
SEQ ID NO: 77
MILDTDYITENGKPVIRVFKKENGEFKIEYDRTFEPYFYALLKDDSAIEDVKKVTAKRHGTVVKVKRA
EKVQKKFLGRPIEVWKLYFNHPQDVPAIRDRIRAHPAVVDIYEYDIPFAKRYLIDKGLIPMEGDEELTMLAFAI
ATLYHEGEEFGTGPILMISYADGSEARVITWKKIDLPYVDVVSTEKEMIKRFLRVVREKDPDVLITYNGDNFD
FAYLKKRCEELGIKFTLGRDGSEPKIQRMGDRFAVEVKGRIHFDLYPVIRRTINLPTYTLEAVYEAVFGKPKEK
VYAEEIAQAWESGEGLERVARYSMEDAKVTYELGREFFPMEAQLSRLIGQSLWDVSRSSTGNLVEWFLLRK
AYKRNELAPNKPDERELARRRGGYAGGYVKEPERGLWDNIVYLDFRSLYPSIIITHNVSPDTLNREGCKEYD
VAPEVGHKFCKDFPGFIPSLLGDLLEERQKIKRKMKATVDPLEKKLLDYRQRRIKILANSFYGYYGYAKARWY
CKECAESVTAWGREYIEMVIRELEEKFGFKVLYADTDGLHATIPGADAETVKKKAKEFLKYINPKLPGLLELEY
EGFYVRGFFVTKKKYAVIDEEGKITTRGLEIVRRDWSEIAKETQARVLEAILKHGDVEEAVRIVKEVTEKLSKY
EVPPEKLVIHIQITRDLRDYKATGPHVAVAKRLAARGVKIRPGTVISYIVLKGSGRIGDRAIPADEFDPTKHRY
DAEYYIENQVLPAVERILKAFGYRKEDLRYQKTKQVGLGAWLKVKGKK
SEQ ID NO: 78
MILDTDYITEDGKPVIRIFKKENGEFKIEYDRTFEPYFYALLKDDSAIEEVKKITAERHGTVVTVKRVE
KVQKKFLGRPVEVWKLYFTHPQDVPAIRDKIREHPAVIDIYEYDIPFAKRYLIDKGLVPMEGDEELKMLAFDIE
TLYHEGEEFAEGPILMISYADEEGARVITWKNVDLPYVDVVSTEREMIKRFLRVVKEKDPDVLITYDGDNFDF
AYLKKRCEKLGINFALGRDGSEPKIQRMGDRFAVEVKGRIHFDLYPVIRRTINLPTYTLEAVYEAVFGQPKEK
VYAEEITTAWETGENLERVARYSMEDAKVTYELGKEFLPMEAQLSRLIGQSLWDVSRSSTGNLVEWFLLRKA
YERNELAPNKPDEKELARRRQSYEGGYVKEPERGLWENIVYLDFRSLYPSIIITHNVSPDTLNREGCKEYDVA
PQVGHRFCKDFPGFIPSLLGDLLEERQKIKKKMKATIDPIERKLLDYRQRLIKILANSYYGYYGYARARWYCK
ECAESVTAWGREYITMTIKEIEEKYGFKVIYSDTDGFFATIPGADAETVKKKAMEFLKYINAKLPGALELEYEG
FYKRGFFVTKKKYAVIDEEGKITTRGLEIVRRDWSEIAKETQARVLEALLKDGDVEKAVRIVKEVTEKLSKYEV
PPEKLVIHKQITRDLKDYKATGPHVAVAKRLAARGVKIRPGTVISYIVLKGSGRIGDRAIPFDEFDPTKHKYDA
EYYIENQVLPAVERILRAFGYRKEDLRYQKTRQVGLSAWLKPKGT
SEQ ID NO: 79
MILDTDYITEDGKPVIRIFKKENGEFKIDYDRNFEPYIYALLKDDSAIEDVKKITAERHGTTVRVVRA
EKVKKKFLGRPIEVWKLYFTHPQDQPAIRDKIKEHPAVVDIYEYDIPFAKRYLIDKGLIPMEGDEELKMLAFAI
ATLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCEEYDV
APQVGHKFCKDFPGFIPSLLGDLLEERQKVKKKMKATIDPIEKKLLDYRQRLIKILANSFYGYYGYAKARWYC
KECAESVTAWGRQYIETTIREIEEKFGFKVLYADTDGFFATIPGADAETVKKKAKEFLDYINAKLPGLLELEYE
GFYKRGFFVTKKKYAVIDEEDKITTRGLEIVRRDWSEIAKETQARVLEAILKHGDVEEAVRIVKEVTEKLSKYQ
VPPQQLAIYQPITRALQDYKAKGPHVAVAKRLAARGIKIRPGTVISYIVLKGSGKIGDRAIPFDEFDPAKHKYD
AEYYIENQVLPAVERILRAFGYRKEDLRYQKTRQVGLGAWLKPKT
SEQ ID NO: 80
MALEEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFANLWGRLEGEERLLWL
YREVERPLSAVLAHMEATGVRLDVAYLRALSLEVAEEIARLEAEVFRLAGHPFNLNSRDQLERVLFDELGLPAI
GKTEKTGKRSTSAAVLEALREAHPIVEKILQYRELTKLKSTYIDPLPDLIHPRTGRLHTRFNQTATATGRLSSS
DPNLQNIPVRTPLGQRIRRTFIAEEGRQLVALDYSQTGLRVLAHLSGDENLIRVFQEGRDIHTETASWMFGV
PREAVDPLMRRAAKTINFGVLYGMSAHRLSQELAIPYEEAQAFIERYFQSFPKVRAWIEKTLEEGRRRGYVET
LFGRRRYVPDLEARVKSVREAAERMAFNMPVQGTAADLMKLAMVKLFPRLEEMGARMLLQVHDELVLEAPK
ERAEAVARLAKEVMEGVYPLAVPLEVEVGIGEDWLSAKE
SEQ ID NO: 81
MILDTDYITENGKPVIRVFKKENGEFKIEYDRTFEPYFYALLKDDSAIEDVKKVTAKRHGTVVKVKRA
EKVQKKFLGRPIEVWKLYFNHPQDVPAIRDRIRAHPAVVDIYEYDIPFAKRYLIDKGLIPMEGDEELTMLAFDI
ETLYHEGEEFGTGPILMISYADGSEARVITWKKIDLPYVDVVSTEKEMIKRFLRVVREKDPDVLITYNGDNFD
FAYLKKRCEELGIKFTLGRDGSEPKIQRMGDRFAVEVKGRIHFDLYPVIRRTINLPTYTLEAVYEAVFGKPKEK
VYAEEIAQAWESGEGLERVARYSMEDAKVTYELGREFFPMEAQLSRLIGQSLWDVSRSSTGNLVEWFLLRK
AYKRNELAPNKPDERELARRRGGYAGGYVKEPERGLWDNIVYLDFRSQYPSIIITHNVSPDTLNREGCKEYD
VAPEVGHKFCKDFPGFIPSLLGDLLEERQKIKRKMKATVDPLEKKLLDYRQRAIKILANSFYGYYGYAKARWY
CKECAESVTAWGREYIEMVIRELEEKFGFKVLYADTDGLHATIPGADAETVKKKAKEFLKYINPKLPGLLELEY
EGFYVRGFFVTKKKYAVIDEEGKITTRGLEIVRRDWSEIAKETQARVLEAILKHGDVEEAVRIVKEVTEKLSKY
EVPPEKLVIHEQITRDLRDYKATGPHVAVAKRLAARGVKIRPGTVISYIVLKGSGRIGDRAIPADEFDPTKHRY
DAEYYIENQVLPAVERILKAFGYRKEDLRYQKTKQVGLGAWLKVKGKK
SEQ ID NO: 82
MILDVDYITEEGKPVIRLFKKENGKFKIEHDRTFRPYIYALLRDDSKIEEVKKITGERHGKIVRIVDVE
KVEKKFLGKPITVWKLYLEHPQDVPTIREKVREHPAVVDIFEYDIPFAKRYLIDKGLIPMEGEEELKILAFDIET
LYHEGEEFGKGPIIMISYADENEAKVITWKNIDLPYVEVVSSEREMIKRFLRIIREKDPDIIVTYNGDSFDFPYL
AKRAEKLGIKLTIGRDGSEPKMQRIGDMTAVEVKGRIHFDLYHVITRTINLPTYTLEAVYEAIFGKPKEKVYAD
EIAKAWESGENLERVAKYSMEDAKATYELGKEFLPMEIQLSRLVGQPLWDVSRSSTGNLVEWFLLRKAYERN
EVAPNKPSEEEYQRRLRESYTGGFVKEPEKGLWENIVYLDFRALYPSIIITHNVSPDTLNLEGCKNYDIAPQV
GHKFCKDIPGFIPSLLGHLLEERQKIKTKMKETQDPIEKILLDYRQKAIKLLANSFYGYYGYAKARWYCKECAE
SVTAWGRKYIELVWKELEEKFGFKVLYIDTDGLYATIPGGESEEIKKKALEFVKYINSKLPGLLELEYEGFYKR
GFFVTKKRYAVIDEEGKVITRGLEIVRRDWSEIAKETQARVLETILKHGDVEEAVRIVKEVIQKLANYEIPPEK
LAIYEQITRPLHEYKAIGPHVAVAKKLAAKGVKIKPGMVIGYIVLRGDGPISNRAILAEEYDPKKHKYDAEYYIE
NQVLPAVLRILEGFGYRKEDLRYQKTRQVGLTSWLNIKKS
SEQ ID NO: 83
MILDTDYITEDGKPVIRIFKKENGEFKIDYDRNFEPYIYALLKDDSAIEDVKKITAERHGTTVRVVRA
EKVKKKFLGRPIEVWKLYFTHPQDQPAIRDKIKEHPAVVDIYEYDIPFAKRYLIDKGLIPMEGDEELKMLAFAI
ATLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCEEYDV
APQVGHKFCKDFPGFIPSLLGDLLEERQKVKKKMKATIDPIEKKLLDYRQRLIKILANSFYGYYGYAKARWYC
KECAESVTAWGRQYIETTIREIEEKFGFKVLYADTDGFFATIPGADAETVKKKAKEFLDYINAKLPGLLELEYE
GFYKRGFFVTKKKYAVIDEEDKITTRGLEIVRRDWSEIAKETQARVLEAILKHGDVEEAVRIVKEVTEKLSKYE
VPTQHLVIHQQITRALNDYKAIGPHVAVAKRLAARGIKIRPGTVISYIVLKGSGRIGDRAIPFDEFDPAKHKYD
AEYYIENQVLPAVERILRAFGYRKEDLRYQKTRQVGLGAWLKPKT
SEQ ID NO: 84
MALEEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFANLWGRLEGEERLLWL
YREVERPLSAVLAHMEATGVRLDVAYLRALSLEVAEEIARLEAEVFRLAGHPFNLNSRDQLERVLFDELGLPAI
GKTEKTGKRSTSAAVLEALREAHPIVEKILQYRELTKLKSTYIDPLPDLIHPRTGRLHTRFNQTATATGRLSSS
DPNLQNIPVRTPLGQRIRRAFIAEEGWLLVALDYSQEGLRVLAHLSGDENLIRVFQEGRDIHTETASWMFGV
PREAVDPLMRRAAKTINFGVLYGMSAHRLSQELAIPYEEAQAFIERYFQSFPKVRAWIEKTLEEGRRRGYVET
LFGRRRYVPDLEARVKSVREAAERMAFNMPVQGTAADLMKLAMVKLFPRLEEMGARMLLQVHDELVLEAPK
ERAEAVARLAKEVMEGVYPLAVPLEVEVGIGEDWLSAKE
SEQ ID NO: 85
MILDTDYITENGKPVIRVFKKENGEFKIEYDRTFEPYFYALLKDDSAIEDVKKVTAKRHGTVVKVKRA
EKVQKKFLSRPIEVWKLYFNHPQDVPAIRDRIRAHPAVVDIYEYDIPFAKRYLIDKGLIPMEGDEELTMLAFDI
ETLYHEGEEFGTGPILMISYADGSEARVITWKKIDLPYVDVVSTEKEMIKRFLRVVREKDPDVLITYNGDNFD
FAYLKKRCEELGIKFTLGRDGSEPKIQRMGDRFAVEVKGRIHFDLYPVIRRTINLPTYTLDAVYEVVFGKPKEK
VYAEEIAQAWKSGEGLERVARYSMEDAKVTYELGREFFPMEAQLSRLIGQSLWDVSRSSTGNLVEWFLLRK
AYKRNELAPNKPDERELARRRGGYAGGYVKEPERGLWDNIVYLDFRSLYPSIIITHNVSPDTLNREGCKEYD
VAPEVGHKFCKDFPGFIPSLLGDLLEERQKIKRKMKATVDPLEKKLLDYRQRAIKILANSFYGYYGYAKARWY
CKECAESVTAWGREYIEMVIRELEEKFGFKVLYADTDGLHATIPGADAETVKKKAKEFLKYIKPKLPGLLELEY
EGFYVRGFFVTKKKYAVIDEEGKITTRGLEIVRRDWSEIAKETQARVLEAILKHGDVEEAVRIVKEVTEKLSKC
EVPPEKLVIHEQITRDLRDYKATGPHVAVAKRLAARGVKIRPGTVISYIVLKGSGRIGDRAIPADEFDPTKHRY
DAEYYIENQVLPAVERILKAFGYRKEDLRYQKTKQVGLGAWLKVKGKK
SEQ ID NO: 86
MILDTDYITEDGKPVIRIFKKENGEFKIDYDRNFEPYIYALLKDDSAIEDVKKITAERHGTTVRVVRA
EKVKKKFLGRPIEVWKLYFTHPQDVPAIRDKIKEHPAVVDIYEYDIPFAKRYLIDKGLIPMEGDEELKMLAFDI
ETLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCEEYDV
APQVGHKFCKDFPGFIPSLLGDLLEERQKVKKKMKATIDPIEKKLLDYRQRAIKILANSFYGYYGYAKARWYC
KECAESVTAWGRQYIETTIREIEEKFGFKVLYADTDGFFATIPGADAETVKKKAKEFLDYINAKLPGLLELEYE
GFYKRGFFVTKKKYAVIDEEDKITTRGLEIVRRDWSEIAKETQARVLEAILKHGDVEEAVRIVKEVTEKLSKYE
VPPEKLVIYEQITRDLKDYKATGPHVAVAKRLAARGIKIRPGTVISYIVLKGSGRIGDRAIPFDEFDPAKHKYD
AEYYIENQVLPAVERILRAFGYRKEDLRYQKTRQVGLGAWLKPKT
SEQ ID NO: 87
MILDTDYITEDGKPVIRIFKKENGEFKIDYDRNFEPYIYALLKDDSAIEDVKKITAERHGTTVRVVRA
EKVKKKFLGRPIEVWKLYFTHPQDVPAIRDKIKEHPAVVDIYEYDIPFAKRYLIDKGLIPMEGDEELKMLAFAI
ATLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCEEYDV
APQVGHKFCKDFPGFIPSLLGDLLEERQKVKKKMKATIDPIEKKLLDYRQRRIKILANSFYGYYGYAKARWYC
KECAESVTAWGRQYIETTIREIEEKFGFKVLYADTDGFFATIPGADAETVKKKAKEFLDYINAKLPGLLELEYE
GFYKRGFFVTKKKYAVIDEEDKITTRGLEIVRRDWSEIAKETQARVLEAILKHGDVEEAVRIVKEVTEKLSKYE
VPPEKLVIYIQITRDLKDYKATGPHVAVAKRLAARGIKIRPGTVISYIVLKGSGRIGDRAIPFDEFDPAKHKYDA
EYYIENQVLPAVERILRAFGYRKEDLRYQKTRQVGLGAWLKPKT
SEQ ID NO: 88
MALEEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFANLWGRLEGEERLLWL
YREVERPLSAVLAHMEATGVRLDVAYLRALSLEVAEEIARLEAEVFRLAGHPFNLNSRDQLERVLFDELGLPAI
GKTEKTGKRSTSAAVLEALREAHPIVEKILQYRELTKLKSTYIDPLPDLIHPRTGRLHTRFNQTATATGRLSSS
DPNLQNIPVRTPLGQRIRRAFIAEEGWLLVALDYSQEGLRVLAHLSGDENLIRVFQEGRDIHTETASWMFGV
PREAVNPMMRRAAKTINFGVLYGMSAHRLSQKLAIPYEEAQAFIERYFQSFPKVRAWIEKTLEEGRRRGYVE
TLFGRRRYVPDLEARVKSVRNAAERRAFNMPVQGTAADLMKLAMVKLFPRLEEMGARMLLQVHDELVLEAP
KERAEAVARLAKEVMEGVYPLAVPLEVEVGIGEDWLSAKE
SEQ ID NO: 89
MALGEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFANLWGRLEGEERLLWL
YREVERPLSAVLAHMEATGVRLDVAYLRALSLEVAEEIARLEAEVFRLAGHPFNLNSRDQLERVLFDELGLPAI
GKTEKTGKRSTSAAVLEALREAHPIVEKILQYRELTKLKSTYIDPLPDLIHPRTGRLHTRFNQTATATGRLSSS
DPNLQNIPVRTPLGQRIRRAFIAEEGWLLVALDYSQEGLRVLAHLSGDENLIRVFQEGRDIHTETASWMFGV
PREAVDPLMRRAAKTINFGVLYGMSAHRLSQVLAIPYEEAQAFIERYFQSFPKVRAWIEKTLEEGRRRGYVET
LFGRRRYVPDLEARVKSVREAAERMAFNMPVQGTAADLMKLAMVKLFPRLEEMGARMLLQVHDELVLEAPK
ERAEAVARLAKEVMEGVYPLAVPLEVEVGIGEDWLSAKE
SEQ ID NO: 90
MILDTDYITENGKPVIRVFKKENGEFKIEYDRTFEPYFYALLKDDSAIEDVKKVTAKRHGTVVKVKRA
EKVQKKFLGRPIEVWKLYFNHPQDVPAIRDRIRAHPAVVDIYEYDIPFAKRYLIDKGLIPMEGDEELTMLAFDI
ETLYHEGEEFGTGPILMISYADGSEARVVTWKKIDLPYVDVVSTEKEMIKRFLRVVREKDPDVLITYNGDNFD
FAYLKKRCEELGIKFTLGRDGSEPKIQRMGDRFAVEVKGRIHFDLYPVIRRTINLPTYTLEAVYEAVFGKPKEK
VYAEEIAQAWESGEGLERVARYSMEDAKVTYELGREFFPMEAQLSRLIGQSLWDVSRSSTGNLVEWFLLRK
AYKRNELAPNKPDERELARRRGGYAGGYVKEPERGLWDNIVYLDFRSLYPSIIITHNVSPDTLNREGCKEYD
VAPEVGHKFCKDFPGFIPSLLGDLLEERQKIKRKMKATVDPLEEKLLDYRQRAIKFLANSFYGYYGYAKARWY
CKECAESVTAWGREYIEMVIRELEEKFGFKVLYADTDGLHATIPGADAETVKKKAKEFLKYINPKLPGLLELEY
EGFYVRGFFVTKKKYAVIDEEGKITTRGLEIVRRDWSEFAKETQARVLEAILKHGDVEEAVRIVKEVTEKLSKY
EVPPEKLVIHEQITRDLRDYKATGPHVAVAKRLAARGVKIRPGTVISYIVLKGSGRIGDRAIPADEFDPTKHRY
DAEYYIENQVLPAVERILKAFGYRKEDLRYQKTKQVGLGAWLKVKGKK
SEQ ID NO: 91
MILDTDYITEDGKPVIRIFKKENGEFKIDYDRNFEPYIYALLKDDSAIEDVKKITAERHGTTVRVVRA
EKVKKKFLGRPIEVWKLYFTHPQDQPAIRDKIKEHPAVVDIYEYDIPFAKRYLIDKGLIPMEGDEELKMLAFAI
ATLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCGEYDV
APQVGHKFCKDFPGFIPSLLGDLLEERQKVKKKMKATIDPIEKKLLDYRQRLIKILANSFYGYYGYAKARWYC
KECAESVTAWGRQYLETTIREIEEKFGFKVLYADTDGFFATIPGADAETVKKKAKEFLDYINAKLPGLLELEYE
GFYKRGFFVTKKKYAVIDEEDKITTRGLEIVRRDWSEIAKETQARVLEAILKHGDVEEAVRIVKEVTEKLSKYE
VPPEKLVIYEQITRDLKDYKATGPHVAVAKRLAARGIKIRPGTVISYIVLKGSGRIGDRAIPFDEFDPAKHRYD
AEYYIENQVLPAVERILRAFGYRKEDLRYQKTRQVGLGAWLKPKT
SEQ ID NO: 92
MILDTDYITEDGKPVIRIFKKENGEFKIDYDRNFEPYIYALLKDDSAIEDVKKITAERHGTTVRVVRA
EKVKKKFLGRPIEVWKLYFTHPQDVPAIRDKIKEHPAVVDIYEYDIPFAKRYLIDKGLIPMEGDEELKMLAFDI
ETLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLYPSIIITHNVSPDTLNREGCEEYDV
APQVGHKFCKDFPGFIPSLLGDLLEERQKVKKKMKATIDPIEKKLLDYRQRLIKILANSFYGYYGYAKARWYC
KECAESVTAWGRQYIETTIREIEEKFGFKVLYADTDGFFATIPGADAETVKKKAKEFLDYINAKLPGLLELEYE
GFYKRGFFVTKKKYAVIDEEDKITTRGLEIVRRDWSEIAKETQARVLEAILKHGDVEEAVRIVKEVTEKLSKYQ
VPPQQLAIYQPITRALQDYKAKGPHVAVAKRLAARGIKIRPGTVISYIVLKGSGKIGDRAIPFDEFDPAKHKYD
AEYYIENQVLPAVERILRAFGYRKEDLRYQKTRQVGLGAWLKPKT
SEQ ID NO: 93
MALEEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFANLWGRLEGEERLLWL
YREVERPLSAVLAHMEATGVRLDVAYLRALSLEVAEEIARLEAEVFRLAGHPFNLNSRDQLERVLFDELGLPAI
GKTEKTGKRSTSAAVLEALREAHPIVEKILQYRELTKLKSTYIDPLPDLIHPRTGRLHTRFNQTATATGRLSSS
DPNLQNIPVRTPLGQRIRRAFIAEEGWLLVALDYSQEGLRVLAHLSGDENLIRVFQEGRDIHTETASWMFGV
PREAVDPLMRRAAKTINFGVLYGMSAHRLSQELAIPYEEAQAFIERYFQSFPKVRAWIEKTLEEGRRRGYVET
LFGRRRYVPDLEARVKSVRRRAERMAFNMPVQGTAADLMKLAMVKLFPRLEEMGARMLLQVHDELVLEAPK
ERAEAVARLAKEVMEGVYPLAVPLEVEVGIGEDWLSAKE
SEQ ID NO: 94
MALEEAPWPPPEGAFVGFVLSRKEPMWADLLALAAARGGRVHRAPEPYKALRDLKEARGLLAKDLS
VLALREGLGLPPGDDPMLLAYLLDPSNTTPEGVARRYGGEWTEEAGERAALSERLFANLWGRLEGEERLLWL
YREVERPLSAVLAHMEATGVRLDVAYLRALSLEVAEEIARLEAEVFRLAGHPFNLNSRDQLERVLFDELGLPAI
GKTEKTGKRSTSAAALEALREAHPIVEKILQYRELTKLKSTYIDPLPDLIHPRTGRLHTRFNQTATATGRLSSS
DPNLQSIPVRTPLGQRIRRAFIAEEGWLLVALDYSQEGLRVLAHLSGDENLIRVFQEGRDIHTETASWMFGV
PREAVNPLMRRAAKTINFGVLYGMSAHRLSQKLAIPYEEAQAFIERYFQSFPKVRAWIEKTLEEGRRRGYVET
LFGRRRYVPDLEARVKSVRQAAERRAFNMPVQGTAADLMKLAMVKLFPRLEEMGARMLLQVHDELVLEAPK
ERAEAVARLAKEVMEGVYPLAVPLEVEVGIGEDWLSAKE
SEQ ID NO: 95
MILDTDYITEDGKPVIRIFKKENGEFKIDYDRNFEPYIYALLKDDSAIEDVKKITAERHGTTVRVVRA
EKVKKKFLGRPIEVWKLYFTHPQDQPAIRDKIKEHPAVVDIYEYDIPFAKRYLIDKGLIPMEGDEELKMLAFAI
ATLYHEGEEFAEGPILMISYADEEGARVITWKNIDLPYVDVVSTEKEMIKRFLKVVKEKDPDVLITYNGDNFD
FAYLKKRSEKLGVKFILGREGSEPKIQRMGDRFAVEVKGRIHFDLYPVIRRTINLPTYTLEAVYEAIFGQPKEK
VYAEEIAQAWETGEGLERVARYSMEDAKVTYELGKEFFPMEAQLSRLVGQSLWDVSRSSTGNLVEWFLLRK
AYERNELAPNKPDERELARRRESYAGGYVKEPERGLWENIVYLDFRSLGPSIIITHNVSPDTLNREGCEEYDV
APQVGHKFCKDFPGFIPSLLGDLLEERQKVKKKMKATIDPIEKKLLDYRQRLIKILANSFYGYYGYAKARWYC
KECAESVTAWGRQYIETTIREIEEKFGFKVLYADTDGFFATIPGADAETVKKKAKEFLDYINAKLPGLLELEYE
GFYKRGFFVTKKKYAVIDEEDKITTRGLEIVRRDWSEIAKETQARVLEAILKHGDVEEAVRIVKEVTEKLSKYE
VPPEKLVIYKQITRDLKDYKATGPHVAVAKRLAARGIKIRPGTVISYIVLKGSGRIGDRAIPFDEFDPAKHKYD
AEYYIENQVLPAVERILRAFGYRKEDLRYQKTRQVGLGAWLKPKT
SEQ ID NO: 96
/5Biosg/GCACTTCGCTTCACCTCTCTGCTTT
SEQ ID NO: 97
eGeCeAeGeAeGeAGG
SEQ ID NO: 98
/5phos/GAeAeGeTeGeC
SEQ ID NO: 99
eGeCeAeGeAeGeAGGTGAAGCGAeAeGeTeGeC
SEQ ID NO: 100
eGeCeAeGeAeGeAGGTGAAGC
SEQ ID NO: 101
/5Phos/rGrUrCrUrGrCrUrUrCrGrCrArCrCrArArArGrCrCmAmUmUmAmGmC
SEQ ID NO: 102
rArCrUrCrCrUrCrGrGrUrGrCrCrUrCrArArUrGrGrGrCrArGrCrArGrCrCrArUrCrArUrCrArUrCrArU
rCrArUrCrArCrArGrCrArGrCrGrGrCrCrUrGrGrUrGrCrCrGrCrGrCrGrGrCrArGrCrCrArUrArUrGrGrCrUrAr
GrCrArUrGrGrUrGrArGrCrArArArGrGrUrGrArArGrArGrGrArUrArArUrArUrGrGrCrCrArUrCrArUrCrArAr
ArGrArArUrUrCrArUrGrCrGrCrUrUrCrArArArGrUrUrCrArUrArUrGrGrArArGrGrUrArGrCrGrUrUrArArUr
GrGrCrCrArCrGrArArUrUrUrGrArArArUrUrGrArArGrGrUrGrArArGrGrCrGrArArGrGrUrCrGrUrCrCrGrUr
ArUrGrArArGrGrCrArCrCrCrArGrArCrCrGrCrArArArArCrUrGrArArArGrUrUrArCrCrArArArGrGrUrGrGrU
rCrCrGrCrUrGrCrCrGrUrUrUrGrCrArUrGrGrGrArUrArUrUrCrUrGrArGrUrCrCrGrCrArGrUrUrUrArUrGrU
rArUrGrGrUrArGrCrArArArGrCrCrUrArUrGrUrUrArArArCrArUrCrCrGrGrCrArGrArUrArUrCrCrCrGrGrAr
UrUrArUrCrUrGrArArArCrUrGrArGrCrUrUrUrCrCrGrGrArArGrGrUrUrUrUrArArArUrGrGrGrArArCrGrUr
GrUrGrArUrGrArArUrUrUrUrGrArArGrArUrGrGrUrGrGrUrGrUrUrGrUrUrArCrCrGrUrUrArCrArCrArGrG
rArUrArGrCrArGrCrCrUrGrCrArGrGrArUrGrGrUrGrArArUrUrUrArUrCrUrArUrArArArGrUrUrArArArCrU
rGrCrGrUrGrGrCrArCrGrArArUrUrUrUrCrCrGrArGrUrGrArUrGrGrUrCrCrGrGrUrUrArUrGrCrArGrArArA
rArArGrArCrCrArUrGrGrGrUrUrGrGrGrArArGrCrArArGrCrArGrCrGrArArCrGrUrArUrGrUrArUrCrCrGrG
rArArGrArUrGrGrCrGrCrArCrUrGrArArArGrGrUrGrArArArUrUrArArArCrArGrCrGrUrCrUrGrArArGrCrUr
GrArArArGrArUrGrGrCrGrGrUrCrArUrUrArUrGrArUrGrCrArGrArArGrUrUrArArArArCrCrArCrCrUrArCr
ArArArGrCrCrArArArArArGrCrCrGrGrUrUrCrArGrCrUrGrCrCrUrGrGrUrGrCrArUrArUrArArUrGrUrUrAr
ArUrArUrCrArArArCrUrGrGrArUrArUrCrArCrCrArGrCrCrArCrArArCrGrArGrGrArUrUrArUrArCrCrArUrU
rGrUrUrGrArArCrArGrUrArUrGrArArCrGrUrGrCrArGrArArGrGrUrCrGrCrCrArUrArGrUrArCrCrGrGrUrG
rGrUrArUrGrGrArUrGrArArCrUrGrUrArUrArArGrUrArArCrUrCrGrArGrArGrArUrCrCrGrGrCrUrGrCrUrA
rArCrArArArGrCrCrCrGrArArArGrGrArArGrCrUrGrArGrUrUrGrGrCrUrGrCrUrGrCrCrArCrCrGrCrUrGrAr
GrCrArArUrArArGrUrCrUrGrCrUrUrCrGrCrArCrCrArArArGrCrCmAmUmUmAmGmC
SEQ ID NO: 103
mAmCmUmCmCmUmCmGmGmUmGmCmCrUrCrArArUrGrGrGrCrArGrCrArGrCrCrArUrCrAr
UrCrArUrCrArUrCrArUrCrArCrArGrCrArGrCrGrGrCrCrUrGrGrUrGrCrCrGrCrGrCrGrGrCrArGrCrCrArUr
ArUrGrGrCrUrArGrCrArUrGrGrUrGrArGrCrArArArGrGrUrGrArArGrArGrGrArUrArArUrArUrGrGrCrCrAr
UrCrArUrCrArArArGrArArUrUrCrArUrGrCrGrCrUrUrCrArArArGrUrUrCrArUrArUrGrGrArArGrGrUrArGr
CrGrUrUrArArUrGrGrCrCrArCrGrArArUrUrUrGrArArArUrUrGrArArGrGrUrGrArArGrGrCrGrArArGrGrUr
CrGrUrCrCrGrUrArUrGrArArGrGrCrArCrCrCrArGrArCrCrGrCrArArArArCrUrGrArArArGrUrUrArCrCrArA
rArGrGrUrGrGrUrCrCrGrCrUrGrCrCrGrUrUrUrGrCrArUrGrGrGrArUrArUrUrCrUrGrArGrUrCrCrGrCrArG
rUrUrUrArUrGrUrArUrGrGrUrArGrCrArArArGrCrCrUrArUrGrUrUrArArArCrArUrCrCrGrGrCrArGrArUrA
rUrCrCrCrGrGrArUrUrArUrCrUrGrArArArCrUrGrArGrCrUrUrUrCrCrGrGrArArGrGrUrUrUrUrArArArUrG
rGrGrArArCrGrUrGrUrGrArUrGrArArUrUrUrUrGrArArGrArUrGrGrUrGrGrUrGrUrUrGrUrUrArCrCrGrUr
UrArCrArCrArGrGrArUrArGrCrArGrCrCrUrGrCrArGrGrArUrGrGrUrGrArArUrUrUrArUrCrUrArUrArArAr
GrUrUrArArArCrUrGrCrGrUrGrGrCrArCrGrArArUrUrUrUrCrCrGrArGrUrGrArUrGrGrUrCrCrGrGrUrUrAr
UrGrCrArGrArArArArArGrArCrCrArUrGrGrGrUrUrGrGrGrArArGrCrArArGrCrArGrCrGrArArCrGrUrArUr
GrUrArUrCrCrGrGrArArGrArUrGrGrCrGrCrArCrUrGrArArArGrGrUrGrArArArUrUrArArArCrArGrCrGrUr
CrUrGrArArGrCrUrGrArArArGrArUrGrGrCrGrGrUrCrArUrUrArUrGrArUrGrCrArGrArArGrUrUrArArArAr
CrCrArCrCrUrArCrArArArGrCrCrArArArArArGrCrCrGrGrUrUrCrArGrCrUrGrCrCrUrGrGrUrGrCrArUrArU
rArArUrGrUrUrArArUrArUrCrArArArCrUrGrGrArUrArUrCrArCrCrArGrCrCrArCrArArCrGrArGrGrArUrUr
ArUrArCrCrArUrUrGrUrUrGrArArCrArGrUrArUrGrArArCrGrUrGrCrArGrArArGrGrUrCrGrCrCrArUrArGr
UrArCrCrGrGrUrGrGrUrArUrGrGrArUrGrArArCrUrGrUrArUrArArGrUrArArCrUrCrGrArGrArGrArUrCrCr
GrGrCrUrGrCrUrArArCrArArArGrCrCrCrGrArArArGrGrArArGrCrUrGrArGrUrUrGrGrCrUrGrCrUrGrCrCr
ArCrCrGrCrUrGrArGrCrArArUrArArGrUrCrUrGrCrUrUrCrGrCrArCrCrArArArGrCrCmAmUmUmAmGmC
SEQ ID NO: 104
rArCrUrCrCrUrCrGrGrUrGrCrCrUrCrA
SEQ ID NO: 105
rArCrUrCrCrUrCrGrGrUrGrCrCrUrCrArArUrGrGrGrCrArGrCrArGrCrCrArUrCrArUrCrArUrCrArU
rCrArUrCrArCrArGrCrArGrCrGrGrCrCrUrGrGrUrGrCrCrGrCrGrCrGrGrCrArGrCrCrArUrArUrGrGrCrUrAr
GrCrArUrGrGrUrGrArGrCrArArArGrGrUrGrArArGrArGrGrArUrArArUrArUrGrGrCrCrArUrCrArUrCrArAr
ArGrArArUrUrCrArUrGrCrGrCrUrUrCrArArArGrUrUrCrArUrArUrGrGrArArGrGrUrArGrCrGrUrUrArArUr
GrGrCrCrArCrGrArArUrUrUrGrArArArUrUrGrArArGrGrUrGrArArGrGrCrGrArArGrGrUrCrGrUrCrCrGrUr
ArUrGrArArGrGrCrArCrCrCrArGrArCrCrGrCrArArArArCrUrGrArArArGrUrUrArCrCrArArArGrGrUrGrGrU
rCrCrGrCrUrGrCrCrGrUrUrUrGrCrArUrGrGrGrArUrArUrUrCrUrGrArGrUrCrCrGrCrArGrUrUrUrArUrGrU
rArUrGrGrUrArGrCrArArArGrCrCrUrArUrGrUrUrArArArCrArUrCrCrGrGrCrArGrArUrArUrCrCrCrGrGrAr
UrUrArUrCrUrGrArArArCrUrGrArGrCrUrUrUrCrCrGrGrArArGrGrUrUrUrUrArArArUrGrGrGrArArCrGrUr
GrUrGrArUrGrArArUrUrUrUrGrArArGrArUrGrGrUrGrGrUrGrUrUrGrUrUrArCrCrGrUrUrArCrArCrArGrG
rArUrArGrCrArGrCrCrUrGrCrArGrGrArUrGrGrUrGrArArUrUrUrArUrCrUrArUrArArArGrUrUrArArArCrU
rGrCrGrUrGrGrCrArCrGrArArUrUrUrUrCrCrGrArGrUrGrArUrGrGrUrCrCrGrGrUrUrArUrGrCrArGrArArA
rArArGrArCrCrArUrGrGrGrUrUrGrGrGrArArGrCrArArGrCrArGrCrGrArArCrGrUrArUrGrUrArUrCrCrGrG
rArArGrArUrGrGrCrGrCrArCrUrGrArArArGrGrUrGrArArArUrUrArArArCrArGrCrGrUrCrUrGrArArGrCrUr
GrArArArGrArUrGrGrCrGrGrUrCrArUrUrArUrGrArUrGrCrArGrArArGrUrUrArArArArCrCrArCrCrUrArCr
ArArArGrCrCrArArArArArGrCrCrGrGrUrUrCrArGrCrUrGrCrCrUrGrGrUrGrCrArUrArUrArArUrGrUrUrAr
ArUrArUrCrArArArCrUrGrGrArUrArUrCrArCrCrArGrCrCrArCrArArCrGrArGrGrArUrUrArUrArCrCrArUrU
rGrUrUrGrArArCrArGrUrArUrGrArArCrGrUrGrCrArGrArArGrGrUrCrGrCrCrArUrArGrUrArCrCrGrGrUrG
rGrUrArUrGrGrArUrGrArArCrUrGrUrArUrArArGrUrArArCrUrCrGrArGrArGrArUrCrCrGrGrCrUrGrCrUrA
rArCrArArArGrCrCrCrGrArArArGrGrArArGrCrUrGrArGrUrUrGrGrCrUrGrCrUrGrCrCrArCrCrGrCrUrGrAr
GrCrArArUrArArGrUrCrUrGrCrUrUrCrGrCrArCrCrArArArGrCrCmAmUmUmAmGmC
SEQ ID NO: 106
rArCrUrCrCrUrCrGrGrUrGrCrCrUrCrArAΨrGrGrGrCrArGrCrArGrCrCrAΨrCrAΨrCrAΨrCrAΨrC
rAΨrCrArCrArGrCrArGrCrGrGrCrCΨrGrGΨrGrCrCrGrCrGrCrGrGrCrArGrCrCrAΨrAΨrGrGrCΨrArGrCr
AΨrGrGΨrGrArGrCrArArArGrGΨrGrArArGrArGrGrAΨrArAΨrAΨrGrGrCrCrAΨrCrAΨrCrArArArGrArA
ΨΨrCrAΨrGrCrGrCΨΨrCrArArArGΨΨrCrAΨrAΨrGrGrArArGrGΨrArGrCrGΨΨrArAΨrGrGrCrCrArCrG
rArAΨΨΨrGrArArAΨΨrGrArArGrGΨrGrArArGrGrGrGrArArGrGΨrCrGΨrCrCrGΨrAΨrGrArArGrGrCrAr
CrCrCrArGrArCrCrGrCrArArArArCΨrGrArArArGΨΨrArCrCrArArArGrGΨrGrGΨrCrCrGrCΨrGrCrCrGΨ
ΨΨrGrCrAΨrGrGrGrAΨrAΨΨrCΨrGrArGΨrCrCrGrCrArGΨΨΨrAΨrGΨrAΨrGrGΨrArGrCrArArArGrCr
CΨrAΨrGΨΨrArArArCrAΨrCrCrGrGrCrArGrAΨrAΨrCrCrCrGrGrAΨΨrAΨrCΨrGrArArArCΨrGrArGrC
ΨΨΨrCrCrGrGrArArGrGΨΨΨΨrArArAΨrGrGrGrArArCrGΨrGΨrGrAΨrGrArAΨΨΨΨrGrArArGrAΨrGr
GΨrGrGΨrGΨΨrGΨΨrArCrCrGYΨrArCrArCrArGrGrAΨrArGrCrArGrCrCΨrGrCrArGrGrAΨrGrGΨrGrA
rAΨΨΨrAΨrCΨrAΨrArArArGΨΨrArArArCΨrGrCrGΨrGrGrCrArCrGrArAΨΨΨΨrCrCrGrArGΨrGrAΨr
GrGΨrCrCrGrGΨΨrAΨrGrCrArGrArArArArArGrArCrCrAΨrGrGrGΨΨrGrGrGrArArGrCrArArGrCrArGrC
rGrArArCrGΨrAΨrGΨrAΨrCrCrGrGrArArGrAΨrGrGrCrGrCrArCΨrGrArArArGrGΨrGrArArAΨΨrArArAr
CrArGrCrGΨrCΨrGrArArGrCΨrGrArArArGrAΨrGrGrGrGrGΨrCrAΨΨrAΨrGrAΨrGrCrArGrArArGΨΨrA
rArArArCrCrArCrCΨrArCrArArArGrCrCrArArArArArGrCrCrGrGΨΨrCrArGrCΨrGrCrCΨrGrGΨrGrCrAΨ
rAΨrArAΨrGΨΨrArAΨrAΨrCrArArArCΨrGrGrAΨrAΨrCrArCrCrArGrCrCrArCrArArCrGrArGrGrAΨΨrA
ΨrArCrCrAΨΨrGΨΨrGrArArCrArGΨrAΨrGrArArCrGΨrGrCrArGrArArGrGΨrCrGrCrCrAΨrArGΨrArCr
CrGrGΨrGrGΨrAΨrGrGrAΨrGrArArCΨrGΨrAΨrArArGΨrArArCΨrCrGrArGrArGrAΨrCrCrGrGrCΨrGr
CΨrArArCrArArArGrCrCrCrGrArArArGrGrArArGrCΨrGrArGΨΨrGrGrCΨrGrCΨrGrCrCrArCrCrGrCΨrGr
ArGrCrArAΨrArArGrUrCrUrGrCrUrUrCrGrCrArCrCrArArArGrCrCmAmUmUmAmGmC
SEQ ID NO: 107
rArCrUrCrCrUrCrGrGrUrGrCrCrUrCrArAm1ΨrGrGrGrCrArGrCrArGrCrCrAm1ΨrCrAm1ΨrCrA
m1ΨrCrAm1ΨrCrAm1ΨrCrArCrArGrCrArGrCrGrGrCrCm1ΨrGrGm1ΨrGrCrCrGrCrGrCrGrGrCrArGrCr
CrAm1ΨrAm1ΨrGrGrCm1ΨrArGrCrAm1ΨrGrGm1ΨrGrArGrCrArArArGrGm1ΨrGrArArGrArGrGrAm1
ΨrArAm1ΨrAm1ΨrGrGrCrCrAm1ΨrCrAm1ΨrCrArArArGrArAm1Ψm1rCrAm1ΨrGrCrGrCm1Ψm1Ψ
rCrArArArGm1Ψm1ΨrCrAm1ΨrAm1ΨrGrGrArArGrGm1ΨrArGrCrGm1Ψm1ΨrArAm1ΨrGrGrCrCrAr
CrGrArAm1Ψm1m1ΨrGrArArAm1Ψm1ΨrGrArArGrGm1ΨrGrArArGrGrCrGrArArGrGm1ΨrCrGm1Ψ
rCrCrGm1ΨrAm1ΨrGrArArGrGrCrArCrCrCrArGrArCrCrGrCrArArArArCm1ΨrGrArArArGm1Ψm1ΨrAr
CrCrArArArGrGm1ΨrGrGm1ΨrCrCrGrCm1ΨrGrCrCrGm1Ψm1Ψm1rGrCrAm1ΨrGrGrGrAm1ΨrAm
1Ψm1ΨrCm1ΨrGrArGm1ΨrCrCrGrCrArGm1Ψm1Ψm1ΨrAm1ΨrGm1ΨrAm1ΨrGrGm1ΨrArGrCrArA
rArGrCrCm1ΨrAm1ΨrGm1Ψm1ΨrArArArCrAm1ΨrCrCrGrGrCrArGrAm1ΨrAm1ΨrCrCrCrGrGrAm1Ψ
m1ΨrAm1ΨrCm1ΨrGrArArArCm1ΨrGrArGrCm1Ψm1Ψm1YrCrCrGrGrArArGrGm1ΨmΨ1m1Ψm1Ψ
rArArAm1ΨrGrGrGrArArCrGm1ΨrGm1ΨrGrAm1ΨrGrArAm1Ψm1Ψm1Ψm1ΨrGrArArGrAm1ΨrGrG
m1ΨrGrGm1ΨrGm1Ψm1ΨrGm1Ψm1ΨrArCrCrGm1Ψm1ΨrArCrArCrArGrGrAm1ΨrArGrCrArGrCrC
m1ΨrGrCrArGrGrAm1ΨrGrGm1ΨrGrArAm1Ψm1Ψm1ΨrAm1ΨrCm1ΨrAm1ΨrArArArGm1Ψm1ΨrA
rArArCm1ΨrGrCrGm1ΨrGrGrCrArCrGrArAm1Ψm1Ψm1Ψm1ΨrCrCrGrArGm1ΨrGrAm1ΨrGrGm1Ψr
CrCrGrGm1Ψm1ΨrAm1ΨrGrCrArGrArArArArArGrArCrCrAm1ΨrGrGrGm1Ψm1ΨrGrGrGrArArGrCrAr
ArGrCrArGrCrGrArArCrGm1ΨrAm1ΨrGm1ΨrAm1ΨrCrCrGrGrArArGrAm1ΨrGrGrCrGrCrArCm1ΨrGr
ArArArGrGm1ΨrGrArArAm1Ψm1ΨrArArArCrArGrCrGm1ΨrCm1ΨrGrArArGrCm1ΨrGrArArArGrAm1
ΨrGrGrCrGrGm1ΨrCrAm1Ψm1ΨrAm1ΨrGrAm1ΨrGrCrArGrArArGm1Ψm1ΨrArArArArCrCrArCrCm1
ΨrArCrArArArGrCrCrArArArArArGrCrCrGrGm1Ψm1ΨrCrArGrCm1ΨrGrCrCm1ΨrGrGm1ΨrGrCrAm1
ΨrAm1ΨrArAm1ΨrGm1Ψm1ΨrArAm1ΨrAm1ΨrCrArArArCm1ΨrGrGrAm1ΨrAm1ΨrCrArCrCrArGrC
rCrArCrArArCrGrArGrGrAm1Ψm1ΨrAm1ΨrArCrCrAm1Ψm1ΨrGm1Ψm1ΨrGrArArCrArGm1ΨrAm1
ΨrGrArArCrGm1ΨrGrCrArGrArArGrGm1ΨrCrGrCrCrAm1ΨrArGm1ΨrArCrCrGrGm1ΨrGrGm1ΨrAm1
ΨrGrGrAm1ΨrGrArArCm1ΨrGm1ΨrAm1ΨrArArGm1ΨrArArCm1ΨrCrGrArGrArGrAm1ΨrCrCrGrGrC
m1ΨrGrCm1ΨrArArCrArArArGrCrCrCrGrArArArGrGrArArGrCm1ΨrGrArGm1Ψm1ΨrGrGrCm1ΨrGrC
m1ΨrGrCrCrArCrCrGrCm1ΨrGrArGrCrArAm1ΨrArArGrUrCrUrGrCrUrUrCrGrCrArCrCrArArArGrCrCm
AmUmUmAmGmC
SEO ID NO: 108
rArCrUrCrCrUrCrGrGrUrGrCrCrUrCrArArUrGrGrGrCrArGrCrArGrCrCrArUrCrArUrCrArUrCrArU
rCrArUrCrArCrArGrCrArGrCrGrGrCrCrUrGrGrUrGrCrCrGrCrGrCrGrGrCrArGrCrCrArUrArUrGrGrCrUrAr
GrCrArUrGrGrUrGrArGrCrArArArGrGrUrGrArArGrArGrGrArUrArArUrArUrGrGrCrCrArUrCrArUrCrArAr
ArGrArArUrUrCrArUrGrCrGrCrUrUrCrArArArGrUrUrCrArUrArUrGrGrArArGrGrUrArGrCrGrUrUrArArUr
GrGrCrCrArCrGrArArUrUrUrGrArArArUrUrGrArArGrGrUrGrArArGrGrCrGrArArGrGrUrCrGrUrCrCrGrUr
ArUrGrArArGrGrCrArCrCrCrArGrArCrCrGrCrArArArArCrUrGrArArArGrUrUrArCrCrArArArGrGrUrGrGrU
rCrCrGrCrUrGrCrCrGrUrUrUrGrCrArUrGrGrGrArUrArUrUrCrUrGrArGrUrCrCrGrCrArGrUrUrUrArUrGrU
rArUrGrGrUrArGrCrArArArGrCrCrUrArUrGrUrUrArArArCrArUrCrCrGrGrCrArGrArUrArUrCrCrCrGrGrAr
UrUrArUrCrUrGrArArArCrUrGrArGrCrUrUrUrCrCrGrGrArArGrGrUrUrUrUrArArArUrGrGrGrArArCrGrUr
GrUrGrArUrGrArArUrUrUrUrGrArArGrArUrGrGrUrGrGrUrGrUrUrGrUrUrArCrCrGrUrUrArCrArCrArGrG
rArUrArGrCrArGrCrCrUrGrCrArGrGrArUrGrGrUrGrArArUrUrUrArUrCrUrArUrArArArGrUrUrArArArCrU
rGrCrGrUrGrGrCrArCrGrArArUrUrUrUrCrCrGrArGrUrGrArUrGrGrUrCrCrGrGrUrUrArUrGrCrArGrArArA
rArArGrArCrCrArUrGrGrGrUrUrGrGrGrArArGrCrArArGrCrArGrCrGrArArCrGrUrArUrGrUrArUrCrCrGrG
rArArGrArUrGrGrCrGrCrArCrUrGrArArArGrGrUrGrArArArUrUrArArArCrArGrCrGrUrCrUrGrArArGrCrUr
GrArArArGrArUrGrGrCrGrGrUrCrArUrUrArUrGrArUrGrCrArGrArArGrUrUrArArArArCrCrArCrCrUrArCr
ArArArGrCrCrArArArArArGrCrCrGrGrUrUrCrArGrCrUrGrCrCrUrGrGrUrGrCrArUrArUrArArUrGrUrUrAr
ArUrArUrCrArArArCrUrGrGrArUrArUrCrArCrCrArGrCrCrArCrArArCrGrArGrGrArUrUrArUrArCrCrArUrU
rGrUrUrGrArArCrArGrUrArUrGrArArCrGrUrGrCrArGrArArGrGrUrCrGrCrCrArUrArGrUrArCrCrGrGrUrG
rGrUrArUrGrGrArUrGrArArCrUrGrUrArUrArArGrUrArArCrUrCrGrArGrArGrArUrCrCrGrGrCrUrGrCrUrA
rArCrArArArGrCrCrCrGrArArArGrGrArArGrCrUrGrArGrUrUrGrGrCrUrGrCrUrGrCrCrArCrCrGrCrUrGrAr
GrCrArArUrArArGrUrCrUrGrCrUrUrCrGrCrArCrCrArArArGrCrCmAmUmUmAmGmC

What is claimed is:

1. A process for producing a single-stranded polynucleotide product having at least one modified nucleotide residue, the process comprising:
a) contacting a template polynucleotide, which comprises a sequence complementary to the single-stranded polynucleotide product, with a pool of at least two segment polynucleotides under conditions to allow annealing of the at least two segment polynucleotides to the template polynucleotide to generate a template polynucleotide with the at least two segment polynucleotides annealed thereto, wherein at least one sequence gap is formed between the at least two segment polynucleotides annealed;
b) extending at least one of the two segment polynucleotides annealed using a pool of nucleoside triphosphates and a polymerase, to fill in the at least one sequence gap to generate at least one extended segment polynucleotide;
c) ligating segment polynucleotide(s) and/or extended segment polynucleotide(s) using a ligase to form the single-stranded polynucleotide product bound to the template polynucleotide in a duplex; and
d) changing the conditions to denature the duplex comprising the single-stranded polynucleotide product and the template polynucleotide, whereby the single-stranded polynucleotide product is produced.

2. The process according to claim 1, wherein step d) further comprises changing the conditions to denature a duplex comprising an impurity polynucleotide and a template polynucleotide, and separating any impurity polynucleotide(s) prior to changing the conditions to denature the duplex comprising the single-stranded polynucleotide product and the template polynucleotide.

3. The process according to claim 1, further comprising separating the single-stranded polynucleotide product.

4. The process according to claim 1, wherein at least one of the at least two segment polynucleotides comprises at least one modified nucleotide residue.

5. The process according to claim 1, wherein at least one of the at least two segment polynucleotides comprises a 5′ phosphate, a 5′ thiophosphate, 5′ dithiophosphate or 5′ methylphosphate.

6. The process according to claim 1, wherein the pool of nucleoside triphosphates consists of: (i) naturally occurring nucleoside triphosphates; (ii) modified nucleoside triphosphates or (iii) naturally occurring nucleoside triphosphates and modified nucleoside triphosphates.

7. The process according to claim 1, wherein the at least one modified nucleotide residue comprises modification of a sugar moiety, modification of a nucleobase and/or modification of a backbone.

8. The process according to claim 1, wherein the ligase ligates a 3′ end and/or a 5′ end of the segment polynucleotide or the extended segment polynucleotide to an adjacent segment polynucleotide or adjacent extended segment polynucleotide to form the single-stranded polynucleotide product.

9. The process according to claim 1, wherein the ligase in step (c) is capable of ligating two oligonucleotides together, wherein one or both junction nucleotides to be joined is a modified nucleotide.

10. The process according to claim 1, wherein the template polynucleotide has a property that allows it to be separated from the single-stranded polynucleotide product.

11. The process according to claim 1, wherein the process additionally comprises a step of recycling the template polynucleotide.

12. The process according to claim 1, wherein the template polynucleotide is a recycled template polynucleotide.

13. The process according to claim 1, wherein the process is semi-continuous or continuous.

14. A process for producing a double-stranded polynucleotide product, the process comprising annealing two complementary single-stranded polynucleotide products, wherein at least one of the two complementary single-stranded polynucleotide products has been produced by the process of claim 1.

15. A process for producing a double-stranded polynucleotide product having at least one modified nucleotide residue, the process comprising:
a) contacting a template polynucleotide, which comprises a sequence complementary to a single-stranded polynucleotide product, with a pool of at least two segment polynucleotides under conditions to allow annealing of the at least two segment polynucleotides to the template polynucleotide to generate a template polynucleotide with the at least two segment polynucleotides annealed thereto, wherein at least one sequence gap is formed between the at least two segment polynucleotides annealed;
b) extending at least one of the two segment polynucleotides annealed using a pool of nucleoside triphosphates and a polymerase, to fill in the at least one sequence gap to generate at least one extended segment polynucleotide;
c) ligating segment polynucleotide(s) and/or extended segment polynucleotide(s) using a ligase to form the single-stranded polynucleotide product bound to the template polynucleotide in a duplex;
d) changing the conditions to denature the duplex comprising the single-stranded polynucleotide product and the template polynucleotide, whereby the single-stranded polynucleotide product is produced; and
e) using the single-stranded polynucleotide product as the template polynucleotide in step a) and repeating steps a) to c) to produce the double-stranded polynucleotide product.

16. The process according to claim 1, wherein at least one of the at least two segment polynucleotides comprises a 5′ phosphate, a 5′ phosphorothioate, 5′ phosphorodithioate or 5′ methylphosphate at the 3′ end of the sequence gap.

17. The process according to claim 1 wherein the at least one modified nucleotide is N1-methyl-pseudouridine.

18. The process of claim 14 wherein the two complementary single-stranded polynucleotide products have been produced by the process of claim 1.


SEQUENCE LISTING (108 sequences)

SEQ ID NO: 1 - DNA, 42 bp, synthetic construct
  source 1..42: mol_type = other DNA
gctaatggctttggtgcgaagcagactgaggcaccgaggagt

SEQ ID NO: 2 - DNA, 10 bp, synthetic construct
  source 1..10: mol_type = other DNA
actcctcggt

SEQ ID NO: 3 - DNA, 27 bp, synthetic construct
  source 1..27: mol_type = other DNA
agtctgcttcgcaccaaagccattagc

SEQ ID NO: 4 - DNA, 26 bp, synthetic construct
  source 1..26: mol_type = other DNA
gtctgcttcgcaccaaagccattagc

SEQ ID NO: 5 - DNA, 18 bp, synthetic construct
  source 1..18: mol_type = other DNA
  modified_base 11: mod_base = m5c
gcaccaaagcncattagc

SEQ ID NO: 6 - skipped

SEQ ID NO: 7 - DNA, 11 bp, synthetic construct
  source 1..11: mol_type = other DNA
actcctcggtg

SEQ ID NO: 8 - DNA, 12 bp, synthetic construct
  source 1..12: mol_type = other DNA
actcctcggtgc

SEQ ID NO: 9 - DNA, 13 bp, synthetic construct
  source 1..13: mol_type = other DNA
actcctcggtgcc

SEQ ID NO: 10 - DNA, 14 bp, synthetic construct
  source 1..14: mol_type = other DNA
actcctcggtgcct

SEQ ID NO: 11 - DNA, 15 bp, synthetic construct
  source 1..15: mol_type = other DNA
actcctcggtgcctc

SEQ ID NO: 12 - DNA, 16 bp, synthetic construct
  source 1..16: mol_type = other DNA
actcctcggtgcctca

SEQ ID NO: 13 - DNA, 17 bp, synthetic construct
  source 1..17: mol_type = other DNA
actcctcggtgcctcag

SEQ ID NO: 14 - DNA, 18 bp, synthetic construct
  source 1..18: mol_type = other DNA
actcctcggtgcctcagt

SEQ ID NO: 15 - DNA, 19 bp, synthetic construct
  source 1..19: mol_type = other DNA
actcctcggtgcctcagtc

SEQ ID NO: 16 - DNA, 20 bp, synthetic construct
  source 1..20: mol_type = other DNA
actcctcggtgcctcagtct

SEQ ID NO: 17 - DNA, 21 bp, synthetic construct
  source 1..21: mol_type = other DNA
actcctcggtgcctcagtctg

SEQ ID NO: 18 - DNA, 22 bp, synthetic construct
  source 1..22: mol_type = other DNA
actcctcggtgcctcagtctgc

SEQ ID NO: 19 - DNA, 23 bp, synthetic construct
  source 1..23: mol_type = other DNA
actcctcggtgcctcagtctgct

SEQ ID NO: 20 - DNA, 24 bp, synthetic construct
  source 1..24: mol_type = other DNA
actcctcggtgcctcagtctgctt

SEQ ID NO: 21 - DNA, 25 bp, synthetic construct
  source 1..25: mol_type = other DNA
actcctcggtgcctcagtctgcttc

SEQ ID NO: 22 - DNA, 26 bp, synthetic construct
  source 1..26: mol_type = other DNA
actcctcggtgcctcagtctgcttcg

SEQ ID NO: 23 - DNA, 27 bp, synthetic construct
  source 1..27: mol_type = other DNA
actcctcggtgcctcagtctgcttcgc

SEQ ID NO: 24 - DNA, 28 bp, synthetic construct
  source 1..28: mol_type = other DNA
actcctcggtgcctcagtctgcttcgca

SEQ ID NO: 25 - DNA, 29 bp, synthetic construct
  source 1..29: mol_type = other DNA
actcctcggtgcctcagtctgcttcgcac

SEQ ID NO: 26 - DNA, 30 bp, synthetic construct
  source 1..30: mol_type = other DNA
actcctcggtgcctcagtctgcttcgcacc

SEQ ID NO: 27 - DNA, 31 bp, synthetic construct
  source 1..31: mol_type = other DNA
actcctcggtgcctcagtctgcttcgcacca

SEQ ID NO: 28 - DNA, 32 bp, synthetic construct
  source 1..32: mol_type = other DNA
actcctcggtgcctcagtctgcttcgcaccaa

SEQ ID NO: 29 - DNA, 33 bp, synthetic construct
  source 1..33: mol_type = other DNA
actcctcggtgcctcagtctgcttcgcaccaaa

SEQ ID NO: 30 - DNA, 34 bp, synthetic construct
  source 1..34: mol_type = other DNA
actcctcggtgcctcagtctgcttcgcaccaaag

SEQ ID NO: 31 - DNA, 35 bp, synthetic construct
  source 1..35: mol_type = other DNA
actcctcggtgcctcagtctgcttcgcaccaaagc

SEQ ID NO: 32 - DNA, 36 bp, synthetic construct
  source 1..36: mol_type = other DNA
actcctcggtgcctcagtctgcttcgcaccaaagcc

SEQ ID NO: 33 - DNA, 37 bp, synthetic construct
  source 1..37: mol_type = other DNA
actcctcggtgcctcagtctgcttcgcaccaaagcca

SEQ ID NO: 34 - DNA, 38 bp, synthetic construct
  source 1..38: mol_type = other DNA
actcctcggtgcctcagtctgcttcgcaccaaagccat

SEQ ID NO: 35 - DNA, 39 bp, synthetic construct
  source 1..39: mol_type = other DNA
actcctcggtgcctcagtctgcttcgcaccaaagccatt

SEQ ID NO: 36 - DNA, 40 bp, synthetic construct
  source 1..40: mol_type = other DNA
actcctcggtgcctcagtctgcttcgcaccaaagccatta

SEQ ID NO: 37 - DNA, 41 bp, synthetic construct
  source 1..41: mol_type = other DNA
actcctcggtgcctcagtctgcttcgcaccaaagccattag

SEQ ID NO: 38 - DNA, 42 bp, synthetic construct
  source 1..42: mol_type = other DNA
actcctcggtgcctcagtctgcttcgcaccaaagccattagc

SEQ ID NO: 39 - protein, 704 aa, Escherichia phage
MIVSDIEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQIAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICH

SEQ ID NO: 40 - protein, 352 aa, Sulfolobus solfataricus
MIVLFVDFDYFYAQVEEVLNPSLKGKPVVVCVFSGRFEDSGAVATANYEARKFGVKAGIPIVEAKKILPNAVYLPMRKEVYQQVSSRIMNLLREYSEKIEIASIDEAYLDISDKVRDYREAYNLGLEIKNKILEKEKITVTVGISKNKVFAKIAADMAKPNGIKVIDDEEVKRLIRELDIADVPGIGNITAEKLKKLGINKLVDTLSIEFDKLKGMIGEAKAKYLISLARDEYNEPIRTRVRKSIGRIVTMKRNSRNLEEIKPYLFRAIEESYYKLDKRIPKAIHVVAVTEDLDIVSRGRTFPHGISKETAYSESVKLLQKILEEDERKIRRIGVRFSKFIEAIGLDKFFDT

SEQ ID NO: 41 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 42 - protein, 898 aa, Enterobacteria phage
MKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 43 - protein, 541 aa, Thermus aquaticus
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 44 - protein, 774 aa, Thermococcus kodakarensis
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 45 - protein, 346 aa, Enterobacteria phage
MNIFNTNPFKAVSFVESAVKKALETSGYLIADCKYDGVRGNIVVDNVAEAAWLSRVSKFIPALEHLNGFDKRWQQLLNDDRCIFPDGFMLDGELMVKGVDFNTGSGLLRTKWVKRDNMGFHLTNVPTKLTPKGREVIDGKFEFHLDPKRLSVRLYAVMPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTSNVKAHGEDFYNGWACQVNYMEATPDGSLRHPSFEKFRGTEDNPQEKM

SEQ ID NO: 46 - DNA, 45 bp, synthetic construct
  source 1..45: mol_type = other DNA
gctaatggctttggtgcgaagcagactgaggcaccgaggagtttt

SEQ ID NO: 47 - DNA, 53 bp, synthetic construct
  source 1..53: mol_type = other DNA
ctaatggctttggtgcgaagcagacctgatgactgaggcaccgaggagttttt

SEQ ID NO: 48 - DNA, 99 bp, synthetic construct
  source 1..99: mol_type = other DNA
ctaatggctttggtgcgaagcagacctcttcacctttgctcaccatttgtagtccatcggatatatctccttcggatcctgaggcaccgaggagttttt

SEQ ID NO: 49 - skipped

SEQ ID NO: 50 - DNA, 16 bp, synthetic construct
  source 1..16: mol_type = other DNA
actcctcggtgcctca

SEQ ID NO: 51-53 - skipped (3 entries)

SEQ ID NO: 54 - DNA, 26 bp, synthetic construct
  source 1..26: mol_type = other DNA
gtctgcttcgcaccaaagccattagc

SEQ ID NO: 55 - DNA, 70 bp, synthetic construct
  source 1..70: mol_type = other DNA
actcctcggtgcctcaggatccgaaggagatatatccgatggactacaaatggtgagcaaaggtgaagag

SEQ ID NO: 56 - protein, 775 aa, Thermococcus sp.
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 57 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCGEYDVAPQVGHKFCKDFPGFIPSLLGDLLEERQKVKKKMKATIDPIEKKLLDYRQRLIGILANSFYGYYGYAKARWYCKECAESVTAWGRQYLETTIREIEEKFGFKVLYADTDGFFATIPGADAETVKKKAKEFLDYINAKLPGLLELEYEGFYKRGFFVTKKKYAVIDEEDKITTVGLEIVRVDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHRYDAEYYIENQVLPAVERILRAFGYRKEDLRYQKTRQVGLGAWLKPKT

SEQ ID NO: 58 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFATKKKYAVIDEEDKITTRGLKMVRRDWSEIAKETQARVLEAILKHGDVEEAVRIVKEVTEKLSKYEVPPEQLVIYQPITKQLHDYRARGPHVSVAKRLAARGIKIRPGTVISYIVPKGSGRIGDRAIPFDEFDPAKHKYDAGYYIENQVLPAVERILRAFGYRKEDLRYQKTRQVGLGAWLKPKT

SEQ ID NO: 59 - protein, 541 aa, synthetic construct
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFAYLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAALEALREAHPIVEKILQYRELTKLKSTYIDPLPDLIHPRTGRLHTRFNQTATATGRLSSSDPNLQNIPVRTPLGQRIRRAFIAEEGWLLVALDYSQEGLRVLAHLSGDENLIRVFQEGRDIHTETASWMFGVPREAVNPMMRRAAKTINFGVLYGMSAHRLSQVLAIPYEEAQAFIERYFQSFPKVRAWIEKTLEEGRRRGYVETLFGRRRYVPDLEARVKSVRNAAERRAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 60 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDQPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLGPSIIITHNVSPDTLNREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHKQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 61 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLRATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 62 - protein, 774 aa, synthetic construct
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYHEGEEFAEGPILMISYADEEGARVITWKNVDLPYVDVVSTEREMIKRFLRVVKEKDPDVLITYDGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLGPSIIITHNVSPDTLNREGCKEYDVAPQVGHRFCKDFPGFIPSLLGDLLEERQKIKKKMKATIDPIERKLLDYRQRLIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRNWSEIAKETQARVLEALLKDGDVEKAVRIVKEVTEKLSKYEVPPEKLVIHKQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 63 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCGEYDVAPQVGHKFCKDFPGFIPSLLGPLLEERQKVKKKMKATIDPIEKKLLDYRQRLIGILANSFYGYYGYAKARWYCKECAESVTAWGRQYLETTIREIEEKFGFKVLYADTDGFFATIPGADAETVKKKAKEFLDYINAKLPGLLELEYEGFYKRGFFVTKKKYAVIDEEDKITTVGLEIVRVDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHRYDAEYYIENQVLPAVERILRAFGYRKEDLRYQKTRQVGLGAWLKPKT

SEQ ID NO: 64 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 65 - protein, 541 aa, synthetic construct
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQEGLRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKRAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 66 - protein, 775 aa, synthetic construct; identical to SEQ ID NO: 41

SEQ ID NO: 67 - protein, 774 aa, synthetic construct
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRRIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHIQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 68 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 69 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLVPSIIVTHNVSPDTLEKEGCKNYDVAPIVGYRFCKDFPGFIPSILGDLIAMRQDIKKKMKSTIDPIEKKMLDYRQRLIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 70 - protein, 541 aa, synthetic construct
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHVEATGVRLDVAYLRALSLEVAEEIARLEAEVFRLAGHPFNLNSRDQLERVLFDELGLPAIGKTEKTGKRSTSAAVLEALREAHAIVEKILQYRELTKLKSTYIDPLPELIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKV

SEQ ID NO: 71 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRRIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 72 - protein, 774 aa, synthetic construct
MILDTDYITEDGKPVIRIFKKENGEFKIEYDRTFEPYFYALLKDDSAIEEVKKITAERHGTVVTVKRVEKVQKKFLGRPVEVWKLYFTHPQDQPAIRDKIREHPAVIDIYEYDIPFAKRYLIDKGLVPMEGDEELKMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLGPSIIITHNVSPDTLNREGCKEYDVAPQVGHRFCKDFPGFIPSLLGDLLEERQKIKKKMKATIDPIERKLLDYRQRLIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 73 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFATKKKYAVIDEEDKITTRGLKMVRRDWSEIAKETQARVLEAILKHGDVEEAVRIVKEVTEKLSKYEVPPEQLVIYQPITKQLHDYRARGPHVSVAKRLAARGIKIRPGTVISYIVPKGSGRIGDRAIPFDEFDPAKHKYDAGYYIENQVLPAVERILRAFGYRKEDLRYQKTRQVGLGAWLKPKT

SEQ ID NO: 74 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLVPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 75 - protein, 541 aa, synthetic construct
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEVGWLLVVLDYSQMGLRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 76 - DNA, 895 bp, synthetic construct
  source 1..895: mol_type = other DNA
ctaatggctttggtgcgaagcagacttattgctcagcggtggcagcagccaactcagcttcctttcgggctttgttagcagccggatctctcgagttacttatacagttcatccataccaccggtactatggcgaccttctgcacgttcatactgttcaacaatggtataatcctcgttgtggctggtgatatccagtttgatattaacattatatgcaccaggcagctgaaccggctttttggctttgtaggtggttttaacttctgcatcataatgaccgccatctttcagcttcagacgctgtttaatttcacctttcagtgcgccatcttccggatacatacgttcgctgcttgcttcccaacccatggtctttttctgcataaccggaccatcactcggaaaattcgtgccacgcagtttaactttatagataaattcaccatcctgcaggctgctatcctgtgtaacggtaacaacaccaccatcttcaaaattcatcacacgttcccatttaaaaccttccggaaagctcagtttcagataatccgggatatctgccggatgtttaacataggctttgctaccatacataaactgcggactcagaatatcccatgcaaacggcagcggaccacctttggtaactttcagttttgcggtctgggtgccttcatacggacgaccttcgccttcaccttcaatttcaaattcgtggccattaacgctaccttccatatgaactttgaagcgcatgaattctttgatgatggccatattatcctcttcacctttgctcaccatgctagccatatggctgccgcgcggcaccaggccgctgctgtgatgatgatgatgatggctgctgcccattgaggcaccgaggagttttt

SEQ ID NO: 77 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRRIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHIQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 78 - protein, 774 aa, synthetic construct
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYHEGEEFAEGPILMISYADEEGARVITWKNVDLPYVDVVSTEREMIKRFLRVVKEKDPDVLITYDGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRLIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 79 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYQVPPQQLAIYQPITRALQDYKAKGPHVAVAKRLAARGIKIRPGTVISYIVLKGSGKIGDRAIPFDEFDPAKHKYDAEYYIENQVLPAVERILRAFGYRKEDLRYQKTRQVGLGAWLKPKT

SEQ ID NO: 80 - protein, 541 aa, synthetic construct
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRTFIAEEGRQLVALDYSQTGLRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 81 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSQYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 82 - protein, 775 aa, synthetic construct
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 83 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPTQHLVIHQQITRALNDYKAIGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 84 - protein, 541 aa, synthetic construct
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQEGLRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 85 - protein, 775 aa, synthetic construct
MILDTDYITENGKPVIRVFKKENGEFKIEYDRTFEPYFYALLKDDSAIEDVKKVTAKRHGTVVKVKRAEKVQKKFLSRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLDAVYEVVFGKPKEKVYAEEIAQAWKSGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYIKPKLPGLLELEYEGFYVRGFFVTKKKYAVIDEEGKITTRGLEIVRRDWSEIAKETQARVLEAILKHGDVEEAVRIVKEVTEKLSKCEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 86 - protein, 773 aa, Thermococcus gorgonarius
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 87 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRRIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYIQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 88 - protein, 541 aa, synthetic construct
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQEGLRVLAHLSGDENLIRVFQEGRDIHTETASWMFGVPREAVNPMMRRAAKTINFGVLYGMSAHRLSQKLAIPYEEAQAFIERYFQSFPKVRAWIEKTLEEGRRRGYVETLFGRRRYVPDLEARVKSVRNAAERRAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 89 - protein, 541 aa, synthetic construct
MALGEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQEGLRVLAHLSGDENLIRVFQEGRDIHTETASWMFGVPREAVDPLMRRAAKTINFGVLYGMSAHRLSQVLAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 90 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVVTWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEEKLLDYRQRAIKFLANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEFAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 91 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCGEYDVAPQVGHKFCKDFPGFIPSLLGDLLEERQKVKKKMKATIDPIEKKLLDYRQRLIKILANSFYGYYGYAKARWYCKECAESVTAWGRQYLETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHRYDAEYYIENQVLPAVERILRAFGYRKEDLRYQKTRQVGLGAWLKPKT

SEQ ID NO: 92 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYQVPPQQLAIYQPITRALQDYKAKGPHVAVAKRLAARGIKIRPGTVISYIVLKGSGKIGDRAIPFDEFDPAKHKYDAEYYIENQVLPAVERILRAFGYRKEDLRYQKTRQVGLGAWLKPKT

SEQ ID NO: 93 - protein, 541 aa, synthetic construct
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQEGLRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRRAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 94 - protein, 541 aa, synthetic construct
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAALEALREAHPIVEKILQYRELTKLKSTYIDPLPDLIHPRTGRLHTRFNQTATATGRLSSSDPNLQSIPVRTPLGQRIRRAFIAEEGWLLVALDYSQEGLRVLAHLSGDENLIRVFQEGRDIHTETASWMFGVPREAVNPLMRRAAKTINFGVLYGMSAHRLSQKLAIPYEEAQAFIERYFQSFPKVRAWIEKTLEEGRRRGYVETLFGRRRYVPDLEARVKSVRQAAERRAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 95 - protein, 773 aa, synthetic construct
MILDTDYITEDGKPVIRIFKKENGEFKIDYDRNFEPYIYALLKDDSAIEDVKKITAERHGTTVRVVRAEKVKKKFLGRPIEVWKLYFTHPQDQPAIRDKIKEHPAVVDIYEYDIPFAKRYLIDKGLIPMEGDEELKMLAFAIATLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLGPSIIITHNVSPDTLNREGCEEYDVAPQVGHKFCKDFPGFIPSLLGDLLEERQKVKKKMKATIDPIEKKLLDYRQRLIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYKQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 96 - DNA, 25 bp, synthetic construct
  source 1..25: mol_type = other DNA
gcacttcgcttcacctctctgcttt

SEQ ID NO: 97-98 - skipped (2 entries)

SEQ ID NO: 99 - DNA, 22 bp, synthetic construct
  source 1..22: mol_type = other DNA
  modified_base 1: mod_base = OTHER; note = 2-O-methoxy-ethyl guanosine
  modified_base 2: mod_base = OTHER; note = 2-O-methoxy-ethyl 5-methylcytosine
  modified_base 3: mod_base = OTHER; note = 2-O-methoxy-ethyl adenosine
  modified_base 4: mod_base = OTHER; note = 2-O-methoxy-ethyl guanosine
  modified_base 5: mod_base = OTHER; note = 2-O-methoxy-ethyl adenosine
  modified_base 6: mod_base = OTHER; note = 2-O-methoxy-ethyl adenosine
  modified_base 7: mod_base = OTHER; note = 2-O-methoxy-ethyl adenosine
  modified_base 18: mod_base = OTHER; note = 2-O-methoxy-ethyl adenosine
  modified_base 19: mod_base = OTHER; note = 2-O-methoxy-ethyl guanosine
  modified_base 20: mod_base = OTHER; note = 2-O-methoxy-ethyl thymidine
  modified_base 21: mod_base = OTHER; note = 2-O-methoxy-ethyl guanosine
  modified_base 22: mod_base = OTHER; note = 2-O-methoxy-ethyl 5-methylcytosine
nnnnnnnggtgaagcgannnnn

SEQ ID NO: 100-108 - skipped (9 entries)